(19) United States
(12) Patent Application Publication
(10) Pub. No.: US 2025/0027134 A1
(43) Pub. Date: Jan. 23, 2025

(54) SCREENING OF CAS NUCLEASES FOR ALTERED NUCLEASE ACTIVITY

(71) Applicant: The Trustees of Princeton University, Princeton, NJ (US)

(72) Inventors: Cameron MYHRVOLD, Princeton, NJ (US); Gaeun KIM, Stanford, CA (US); Jon ARIZTI SANZ, Cambridge, MA (US); Ryan MCNULTY, Ringoes, NJ (US)

(73) Assignee: The Trustees of Princeton University, Princeton, NJ (US)

(21) Appl. No.: 18/710,260

(22) PCT Filed: Nov. 15, 2022
(86) PCT No.: PCT/US2022/049950

§ 371 Date: May 15, 2024

Related U.S. Application Data
(60) Provisional application No. 63/279,303, filed on Nov. 15, 2021.

(51) Int. Cl. C12Q 1/34

(52) U.S. Cl. CPC C12Q 1/34

(57) ABSTRACT

Described herein is a method of screening for a nuclease with an altered nuclease activity comprising; a) forming a first compartmentalizing reaction comprising a carrier and a variant nuclease template nucleic acid comprising a coding region for a variant nuclease, and amplifying said coding region for said variant nuclease, to obtain variant nuclease encoding nucleic acid; b) forming a second compartmentalizing reaction comprising said variant nuclease encoding nucleic acid, and performing an in vitro transcription and translation reaction to obtain variant nuclease polypeptides; and c) forming a third compartmentalizing reaction comprising said variant nuclease polypeptides, and assaying said variant nuclease polypeptides for the altered nuclease activity of said nuclease.

### CROSS-REFERENCE TO RELATED APPLICATIONS

This application claims the benefit of U.S. Provisional Application No. 63/279,303 filed on Nov. 15, 2021, which is herein incorporated by reference in its entirety.

### STATEMENT OF GOVERNMENT SPONSORED RESEARCH

This invention was made with government support under Grant No. D18AC00006 awarded by the Defense Advanced Research Projects Agency (DARPA). The government has certain rights in the invention.

### BACKGROUND

CRISPR-Cas (Clustered Regularly Interspaced Short Palindromic Repeats-CRISPR associated protein) systems protect bacteria and archaea from invasion by foreign genetic elements. Nucleases are important tools for research and genome engineering. Cas13 is the sole member of Class 2 CRISPR-Cas systems that exclusively target single-stranded RNA (ssRNA). A complete Cas13 effector complex consists of a Cas13 protein and a CRISPR RNA (crRNA). crRNA contains a direct repeat (DR) specific to a Cas13 protein and a spacer complementary to a target RNA.

As with other Class 2 CRISPR enzymes, Cas13 protein has a bi-lobed architecture, containing a nuclease (NUC) lobe and a crRNA recognition (REC) lobe. Two higher eukaryotes and prokaryotes nucleotide (HEPN)-binding domains in the NUC lobe are responsible for the complex's RNase activity. The function and structure of HEPN domains—and hence their RNase activity—vary widely across Cas13 orthologs. Upon recognition of target RNA, HEPN domains initiate cis cleavage, and remain in an active conformation to then carry out trans cleavage of adjacent ssRNA.

Cas13 is an RNA-targeting CRISPR enzyme that exhibits both on-target (cis) and off-target (trans) cleavage activity. Cas12 also exhibits both cis and trans cleavage activity. The field possesses a limited understanding of the features that account for such variation, owing to the lack of comprehensive screening or directed evolution studies for Cas12 and Cas13. There is a need for methods of engineering novel nuclease variants having selectively enhanced or decreased cis or trans nuclease activity, and screening for structural causes of variation in nuclease activity.

### SUMMARY

The present disclosure is a method of engineering novel variants and screening for structural causes of variation in nuclease activity among Cas nucleases with selectively enhanced or decreased cis or trans nuclease activity. The present disclosure methods are useful for screening nucleases such as Cas12 and Cas13 orthologs, as described herein.

A high-throughput screen of Cas protein variants will expand the enzyme's uses in RNA-oriented applications. Cell-based approaches to enzyme screening have inherent limitations such as non-specific interactions between cellular components, restricted throughput, and complex transfection protocols. Cell-free methods may be superior to cell-based methods because it generally has an advantage in speed and scalability. Cell-free transcription-translation (TXTL) systems can measure the DNA or RNA cleavage dynamics of CRISPR effectors to identify or validate Cas13 variant and orthologs having altered binding dynamics or catalytic activity. Cell-free TXTL then can be a suitable platform for screening Cas13 variants for altered nuclease activity.

The present disclosure method can utilize a compartmentalized cell-free variant screening assay in well-plates or double emulsion (DE) droplets, wherein each reaction has differential RNase activity pertaining to a cis and trans cutting as measured by fluorescent reporters. DE droplets can compartmentalize enzyme evolution and single-cell analysis to further increase screening throughput in a cell-free manner. A DE droplet system is particularly advantageous for establishing direct links between sequence and phenotype because it generates monodisperse, fluorescent-activated cell sorting (FACS)-compatible compartmentalized reactions. Each water/oil/water droplet contains a single variant of interest and can accommodate a range of biologically relevant reagents. Unlike traditional single-emulsions, each DE droplet can undergo both fluorescence-based phenotyping and downstream sequencing, circumventing the expense of fluorescence activated droplet sorting (FADS) and the non-specific nature of pooled sequencing. DE droplets can be clustered by fluorescence through FACS, support RT-qPCR, encapsulate mammalian cells, and sort variants with single-droplet precision. The present disclosure provides methods amounting to the field's first large-scale screen of Cas13 and Cas12 enzyme orthologs.

The present disclosure method uses cell-free transcription-translation (TXTL) for high-throughput, rapid expression and screening of Cas variants. One method of nuclease activity screening is to load thousands of variants into individual DE droplets, along with quenched fluorescent reporters and fluorescent protein-expressing plasmid (proxies for trans and cis cleavage, respectively). Individual fluorescence levels may be used to indicate the nuclease activity of a particular variant nuclease. After screening, highly active variants can be enriched through flow cytometry and subjected to next-generation sequencing. The consequent structure-function relationships may inform the design of new variants, which can be optimized for and validated through Cas12- or Cas13-based diagnostic assays. Under this cell-free transcription-translation system, variants are screened separately in parallel in a high-throughput method because each reaction is compartmentalized in an individual emulsion.

Described herein in one aspect is a method of screening for a nuclease with an altered nuclease activity comprising; a) forming a first compartmentalizing reaction comprising a carrier and a variant nuclease template nucleic acid comprising a coding region for a variant nuclease, and amplifying said coding region for said variant nuclease, to obtain variant nuclease encoding nucleic acid; b) forming a second compartmentalizing reaction comprising said variant nuclease encoding nucleic acid, performing an in vitro transcription and translation reaction to obtain variant nuclease polypeptides, and assaying said variant nuclease polypeptides for the altered nuclease activity of said nuclease. The nuclease may be a DNA nuclease or an RNA nuclease. The nuclease may be a Cas12a, Cas12b, Cas12d, Cas13a, a Cas13b, a Cas13d, a CasRx, or another Cas nuclease. The nuclease may be a Cas nuclease having collateral nuclease activity, such as Leptotrichia wadei Cas13 (LwaCas13) or Leptotrichia buccalis Cas13 (LbuCas13) nuclease. The nuclease may be a bacterial nuclease of the Leptotrichia genus. The nuclease may be a nuclease originating from Leptotrichia wadei, Leptotrichia buccalis, Leptotrichia shahii, Leptotrichia massiliensis, Leptotrichia trevisanii, Herbinix hemicellulosilytica, and Escherichia coli.

In other aspects, the nuclease is Ruminococcus flavefaciens XPD3002 (RfxCas13d also known as “CasRx”), or Prevotella sp. P5-125 (PspCas13b).

The nuclease may comprise an amino acid sequence at least 85%, 90%, 95%, 99%, or be identical to the amino acid sequence set forth in SEQ ID NO: 1 or SEQ ID NO: 3. The nuclease may comprise a nucleic acid sequence at least 85%, 90%, 95%, 99%, or be identical to the nucleic acid sequence set forth in SEQ ID NO: 2 or SEQ ID NO: 4. The nuclease may be a variant Cas13 nuclease of a type VI-A CRISPR-Cas system and is characterized by cleavage activity of a single-stranded RNA.

In other embodiments of the present disclosure, the nuclease may comprise an amino acid sequence at least 85%, 90%, 95%, 99%, or be identical to the amino acid sequence set forth in SEQ ID NO: 5 or SEQ ID NO:7. The nuclease may comprise a nucleic acid sequence at least 85%, 90%, 95%, 99%, or be identical to the nucleic acid sequence set forth in SEQ ID NO:6 or SEQ ID NO: 8.

Described herein in one aspect is a method of screening for a nuclease with an altered nuclease activity, wherein a first compartmentalizing reaction comprises one or more of dNTPs, a polymerase, and primers complementary to said coding region for a variant nuclease. A first compartmentalizing reaction may amplify a coding region for a variant nuclease as performed using a polymerase chain reaction. A first compartmentalizing reaction may amplify a coding region for a variant nuclease as performed using isothermal amplification. A first compartmentalizing reaction may comprise a guide nucleic acid. Said guide nucleic acid may be a RNA, may be from 40 to 100 nucleotides in length, and may comprise one or more of; a non-natural internucleoside linkage, a nucleic acid mimetic, a modified sugar moiety, and a modified nucleobase. Said variant nuclease encoding nucleic acid may be complexed to said carrier after amplifying. The variant nuclease encoding nucleic acid may be complexed to said carrier by a non-covalent association.

Said guide nucleic acid may be complexed to a carrier molecule, may be covalently bound to said carrier molecule, and may be non-covalently associated to said carrier molecule. Said carrier molecule may be a bead; may be a bead selected from the list consisting of magnetic material, glass, polyacrylamide, polystyrene, protein A, protein G, streptavidin, antibodies, and silanized material; and may be a magnetic bead.

Said method of screening for a nuclease with an altered nuclease activity may also comprise forming a second compartmentalizing reaction, wherein said second compartmentalizing reaction comprises a variant nuclease polypeptide binding to a guide nucleic acid. Said second compartmentalizing reaction may be a cell-free transcription-translation system comprising an in vitro transcription and translation reaction. Said second compartmentalizing reaction may assay said variant nuclease polypeptides for altered nuclease activity of said nuclease. An assay for screening for a nuclease with an altered nuclease activity may alternatively be performed in a third compartmentalizing reaction, wherein said third compartmentalizing reaction comprises said variant nuclease polypeptides and fluorescence reporter system. Said assay for screening for a nuclease with an altered nuclease activity may comprise a fluorescent screening, for example flow cytometry or fluoresce microscopy. Said assay for screening for a nuclease with an altered nuclease activity may comprise sequencing said variant nuclease template nucleic acid. Said assay for screening for a nuclease with an altered nuclease activity may detect an increase or a decrease in nuclease activity. Detection of altered nuclease activity may comprise trans nuclease activity and cis nuclease activity. Said altered nuclease activity may be determined kinetically. Said first and second compartmentalizing reaction may comprise or consist of an oil and water emulsion or an oil and water double emulsion. Said third compartmentalizing reaction may also comprise or consist of an oil and water emulsion or an oil and water double emulsion.

Described herein in one aspect is a variant nuclease produced by compartmentalizing reactions method illustrated herein. Said variant nuclease produced may be a variant Leptotrichia wadei Cas13 (LwaCas13) nuclease or a variant Leptotrichia buccalis Cas13 (LbuCas13) nuclease. Said variant nuclease produced may be a variant Ruminococcus flavefaciens Cas13d (RfxCas13d or CasRx) nuclease or a variant Prevotella sp. P5-125 (PspCas13b) nuclease. Said variant nuclease produced may comprise one or more amino acid sequence modifications compared to an unaltered LwaCas13, LbuCas13, RfxCas13. PspCas13, SEQ ID NO: 1, SEQ ID NO: 3, SEQ ID NO: 5, or SEQ ID NO:7. Said variant nuclease produced may comprise one or more nucleic acid sequence modifications compared to an unaltered LwaCas13, LbuCas13, SEQ ID NO: 2, and SEQ ID NO: 4 . . . . Said variant nuclease produced may comprise one or more nucleic acid sequence modifications compared to an unaltered, RfxCas13, PspCas13, SEQ ID NO: 6, or SEQ ID NO: 8. Said variant nuclease produced may comprise of amino acid sequence modifications or nucleic acid modifications in one or more of a HEPN-1 domain, a HEPN-2 domain, a Helical-1 domain, a Helical-2 domain, a Cas13a switch region, a Cas13 nuclease lobe, a Cas13 recognition lobe, a Cas13 linker region, a NTD, a Monolith region, a Cas13 catalytic site, RNA binding domain, and combinations thereof. A produced variant nuclease may form a nuclease complex comprising of a guide RNA and said variant LwaCas13 LbuCas13, RfxCas13, or PspCas13.

### BRIEF DESCRIPTION OF THE SEQUENCES

SEQ ID NO: 1 is the amino acid sequence of LwaCas13a.

SEQ ID NO:2 is the nucleic acid sequence encoding LwaCas13a.

SEQ ID NO:3 is the amino acid sequence of LbuCas13a.

SEQ ID NO:4 is the nucleic acid sequence encoding LbuCas13a.

SEQ ID NO:5 is the amino acid sequence of RfxCas13d (CasRx).

SEQ ID NO:6 is the nucleic acid sequence encoding RfxCas13d (CasRx).

SEQ ID NO:7 is the amino acid sequence of PspCas13b.

SEQ ID NO:8 is the nucleic acid sequence encoding PspCas13b

### BRIEF DESCRIPTION OF THE DRAWINGS

The novel features described herein are set forth with particularity in the appended claims. A better understanding of the features and advantages of the features described herein are obtained by reference to the following detailed description that sets forth illustrative examples, in which the principles of the features described herein are utilized, and the accompanying drawings of which:

FIG. 1 illustrates a method of high-throughput nuclease variant screening using compartmentalized emulsions.

FIG. 2 illustrates the method of variant nuclease DNA amplification in a first emulsion.

FIG. 3 illustrates a method of cell-free nuclease transcription and translation in a second emulsion.

FIG. 4 illustrates a method of measuring activity of variant nucleases by fluorescence of a third emulsion.

FIG. 5 illustrates a method of fluorescent measuring and evaluating nuclease variants for altered nuclease activity.

FIG. 6 illustrates a method of making variant Cas13a nucleases using an oligonucleotide library and plasmid mutagenesis.

FIG. 7 illustrates a method of oligonucleotide amplification using rolling circle amplification (RCA).

### DETAILED DESCRIPTION

Described herein is a method of screening for a nuclease with an altered nuclease activity comprising; a) forming a first emulsion comprising a carrier molecule complexed to a variant nuclease template nucleic acid comprising a coding region for a variant nuclease, and amplifying said coding region for said variant nuclease, to obtain variant nuclease encoding nucleic acid; b) forming a second emulsion comprising said variant nuclease encoding nucleic acid, and performing an in vitro transcription and translation reaction to obtain variant nuclease polypeptides; and c) assaying said variant nuclease polypeptides for the altered nuclease activity of said nuclease.

The present disclosure describes a variant of Leptotrichia wadei Cas13 (LwaCas13) nuclease, wherein said variant LwaCas13 nuclease comprises an altered nuclease activity compared to an unaltered LwaCas13.

The present disclosure also describes a variant Leptotrichia buccalis Cas13 (LbuCas13) nuclease, wherein said variant LbuCas13 nuclease comprises an altered nuclease activity compared to an unaltered LbuCas13.

The present disclosure provides a variant Ruminococcus flavefaciens XPD3002 (RfxCas13d or CasRx) nuclease, wherein said variant nuclease comprises an altered nuclease activity compared to an unaltered RfxCas13d (CasRx).

The present disclosure provides a variant Prevotella sp. P5-125 (PspCas13b) nuclease, wherein said variant nuclease comprises an altered nuclease activity compared to an unaltered PspCas13b.

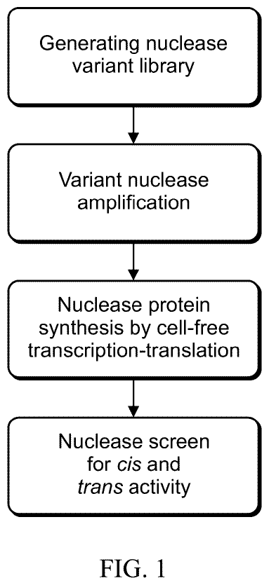

A general method of screening for a nuclease with an altered nuclease activity according to this description is illustrated in FIG. 1. Said method comprises generating a library of variant nucleases; forming a compartmentalized reaction comprising a carrier molecule complexed to a variant nuclease template nucleic acid comprising a coding region for a variant nuclease, and amplifying said coding region for said variant nuclease, to obtain variant nuclease encoding nucleic acid; forming a second compartmentalized reaction comprising said variant nuclease encoding nucleic acid, and performing an in vitro transcription and translation reaction to obtain variant nuclease polypeptides; and forming a third compartmentalized reaction comprising said variant nuclease polypeptides, and assaying said variant nuclease polypeptides for the altered nuclease activity of said variant nuclease polypeptides. Said second and third compartmentalized reaction can optionally be combined into one cell-free transcription-translation and subsequent screening reaction.

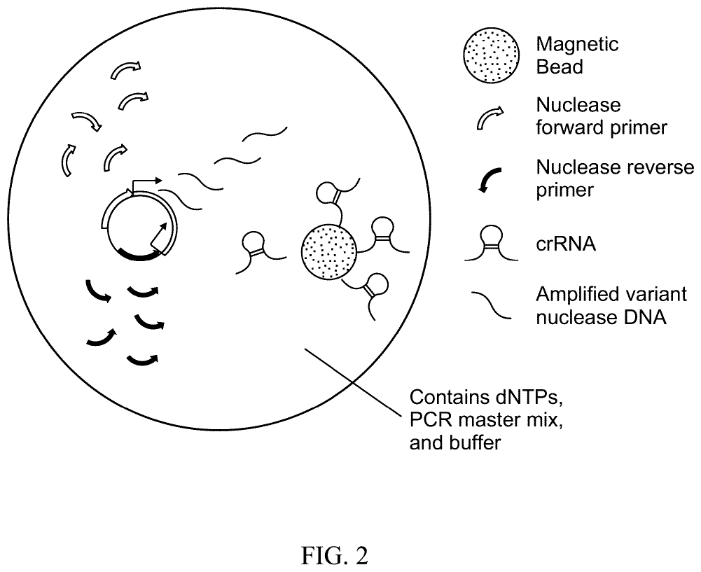

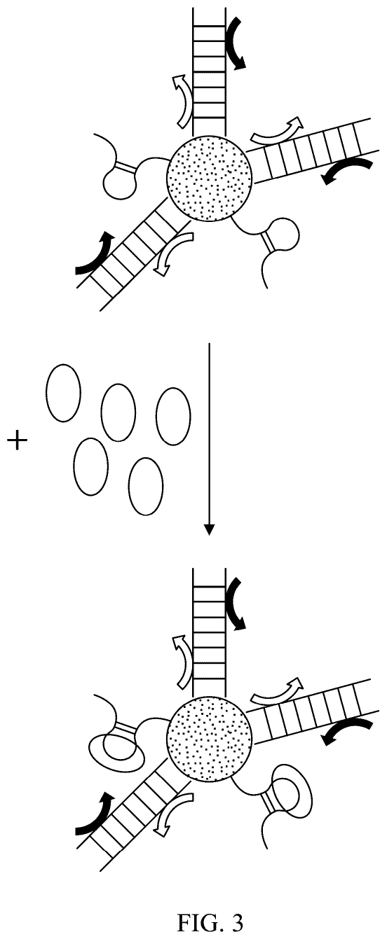

FIG. 2 illustrates the method of Cas DNA amplification in the first compartmentalized reaction. The first compartmentalized reaction may additionally comprise a secondary purification step after Cas DNA amplification. The first compartmentalized reaction comprises one or more reagents including but not limited to dNTPs, PCR buffer, PCR polymerase, variant nuclease primers, variant nuclease template DNA, and a bead for purification of variant RNA. Said first compartmentalized reaction generates variant RNA from said variant nuclease template DNA, which can be generated through PCR or isothermal amplification. FIG. 2 can alternatively comprise one or more reagents for isothermal amplification instead of PCR.

FIG. 3 illustrates a method of variant nuclease transcription and translation in the second compartmentalized reaction for high-throughput and rapid expression of a polypeptide of said variant nuclease. Nuclease polypeptides may then bind to crRNA complexed to a carrier molecule for purposes of separation and purification.

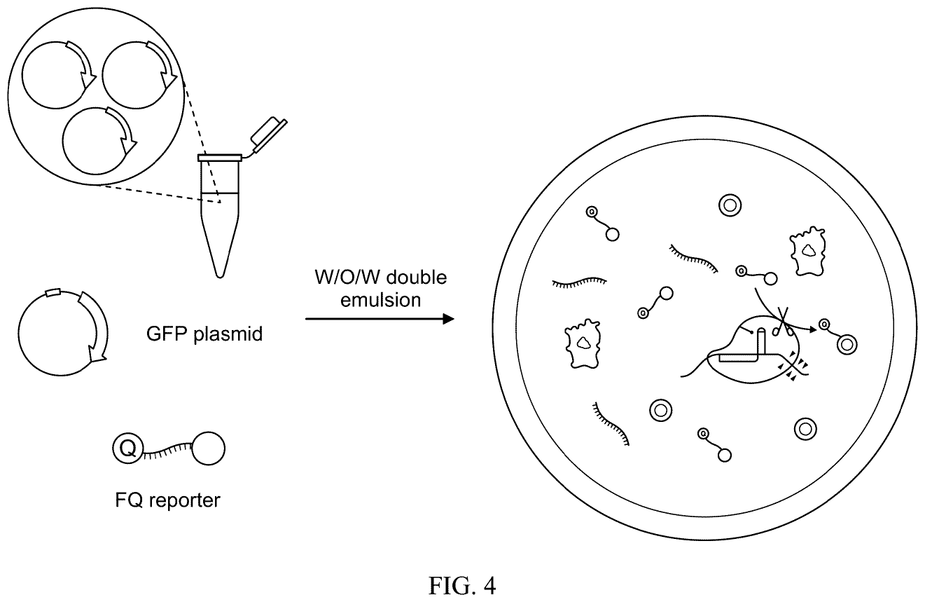

FIG. 4 illustrates a method of measuring Cas nuclease activity in the third compartmentalized reaction by using a fluorescent reporter. Thousands of variants may be loaded into individual double emulsion (DE) droplets, along with quenched fluorescent reporters and fluorescent protein-expressing plasmid (proxies for trans and cis cleavage, respectively). These individual compartmentalized reactions allow fluorescent screening of altered fluorescent expression, altered fluorescent quenching, and altered fluorescent quantification on individual variant nucleases. Each compartmental reaction may be in a cell or cell-free for high-throughput screening. Water-oil-water double emulsions are known in the field, for example the emulsion droplets illustrated in Zinchenko et al. “One in a Million: Flow Cytometric Sorting of Single Cell-Lysate Assays in Monodisperse Picolitre Double Emulsion Droplets for Directed Evolution,” Analytical Chem. 2014, 86, 5, 2526-2533 (2014).

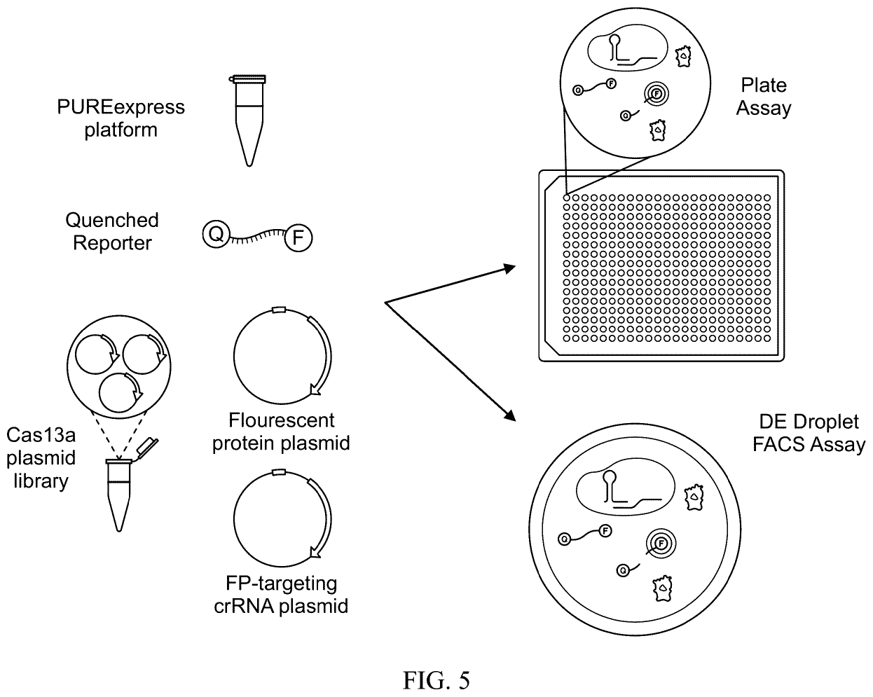

FIG. 5 illustrates a method of fluorescent screening of Cas13 variants for altered cis and trans RNase activity. Each reaction contains, fluorescent protein-expressing plasmid, crRNA-expressing plasmid, a member of a Cas13a variant library, FQ reporter, and PURExpress. Reactions can be carried out either in a 6, 12, 24, 48, 96, 384, or 1536-well plate (2 to 100 microliters) or in DE droplets (15 to 30 picoliters). Reaction products are then screened for variant cis and trans activity levels using FACS analysis or another fluorescent expression assay.

### Certain Definitions

In the following description, certain specific details are set forth in order to provide a thorough understanding of various embodiments. However, one skilled in the art will understand that the embodiments provided may be practiced without these details. Unless the context requires otherwise, throughout the specification and claims which follow, the word “comprise” and variations thereof, such as, “comprises” and “comprising” are to be construed in an open, inclusive sense, that is, as “including, but not limited to.” As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise. Further, headings provided herein are for convenience only and do not interpret the scope or meaning of the claimed embodiments.

As used herein the term “about” refers to an amount that is near the stated amount by 10% or less.

The terms “polypeptide” and “protein” are used interchangeably to refer to a polymer of amino acid residues, and are not limited to a minimum length. Polypeptides, including the provided nucleases and other peptides, e.g., linkers and binding peptides, may include amino acid residues including natural and/or non-natural amino acid residues. The terms also include post-expression modifications of the polypeptide, for example, glycosylation, sialylation, acetylation, phosphorylation, and the like. In some aspects, the polypeptides may contain modifications with respect to a native or natural sequence, as long as the protein maintains the desired activity. These modifications may be deliberate, as through site-directed mutagenesis, or may be accidental, such as through mutations of hosts which produce the proteins or errors due to PCR amplification.

Percent (%) sequence identity with respect to a reference polypeptide sequence is the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y, where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

Amino acid sequence variants of the polypeptides provided herein are contemplated. A variant typically differs from a polypeptide specifically disclosed herein in one or more substitutions, deletions, additions and/or insertions. Such variants can be naturally occurring or can be synthetically generated, for example, by modifying one or more of the above polypeptide sequences of the invention and evaluating one or more biological activities of the polypeptide as described herein and/or using any of a number of known techniques. For example, it may be desirable to improve the binding affinity and/or other biological properties of a polypeptide. Amino acid sequence variants of polypeptides may be prepared by introducing appropriate modifications into the nucleotide sequence encoding the polypeptide, or by peptide synthesis. Such modifications include, for example, deletions from, and/or insertions into and/or substitutions of residues within the amino acid sequences of a protein. Any combination of deletion, insertion, and substitution can be made to arrive at the final construct, provided that the final construct possesses the desired characteristics, (e.g., desired nuclease activity). Any of the polypeptides described herein, including the Cas13, Cas12, or CasRx polypeptides that are described by a SEQ ID NO can be at least about 80%, 85%, 90%, 95%, 97%, 98%, 99%, or can be identical to the referenced SEQ ID NO.

The polypeptides described herein can be encoded by a nucleic acid. A nucleic acid is a type of polynucleotide comprising two or more nucleotide bases. In certain embodiments, the nucleic acid is a component of a vector that can be used to transfer the polypeptide encoding polynucleotide into a cell. As used herein, the term “vector” refers to a nucleic acid molecule capable of transporting another nucleic acid to which it has been linked. One type of vector is a genomic integrated vector, or “integrated vector,” which can become integrated into the chromosomal DNA of the host cell. Another type of vector is an “episomal” vector, e.g., a nucleic acid capable of extra-chromosomal replication. Vectors capable of directing the expression of genes to which they are operatively linked are referred to herein as “expression vectors.” Suitable vectors comprise plasmids, bacterial artificial chromosomes, yeast artificial chromosomes, viral vectors and the like. In the expression vectors regulatory elements such as promoters, enhancers, polyadenylation signals for use in controlling transcription can be derived from mammalian, microbial, viral or insect genes. The ability to replicate in a host, usually conferred by an origin of replication, and a selection gene to facilitate recognition of transformants may additionally be incorporated. Vectors derived from viruses, such as lentiviruses, retroviruses, adenoviruses, adeno-associated viruses, and the like, may be employed. Plasmid vectors can be linearized for integration into a chromosomal location. Vectors can comprise sequences that direct site-specific integration into a defined location or restricted set of sites in the genome (e.g., AttP-AttB recombination). Additionally, vectors can comprise sequences derived from transposable elements.

As used herein, the terms “homologous,” “homology,” or “percent homology” when used herein to describe to an amino acid sequence or a nucleic acid sequence, relative to a reference sequence, can be determined using the formula described by Karlin and Altschul (Proc. Natl. Acad. Sci. USA 87: 2264-2268, 1990, modified as in Proc. Natl. Acad. Sci. USA 90: 5873-5877, 1993). Such a formula is incorporated into the basic local alignment search tool (BLAST) programs of Altschul et al. (J. Mol. Biol. 215: 403-410, 1990). Percent homology of sequences can be determined using the most recent version of BLAST, as of the filing date of this application.

The nucleic acids encoding the nucleases described herein can be used to infect, transfect, transform, or otherwise render a suitable cell transgenic for the nucleic acid, thus enabling the production of nuclease polypeptides for commercial, diagnostic, or therapeutic uses. Standard cell lines and methods for the production of polypeptides from a large scale cell culture are known in the art.

The terms “DNA regulatory sequences,” “control elements,” and “regulatory elements,” used interchangeably herein, refer to transcriptional and translational control sequences, such as promoters, enhancers, polyadenylation signals, terminators, protein degradation signals, and the like, that provide for and/or regulate transcription of a non-coding sequence (e.g., Cas13 guide RNA) or a coding sequence (e.g., a Cas13 protein coding sequence) and/or regulate translation of an encoded polypeptide.

As used herein, a “promoter sequence” is a DNA regulatory region capable of binding RNA polymerase and initiating transcription of a downstream (3′ direction) coding or non-coding sequence. For purposes of the present disclosure, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site, as well as protein binding domains responsible for the binding of RNA polymerase. Eukaryotic promoters will often, but not always, contain “TATA” boxes and “CAT” boxes. Various promoters, including inducible promoters, may be used to drive the various vectors of the present disclosure.

The term “naturally-occurring” or “unmodified” or “wild type” as used herein as applied to a nucleic acid, a polypeptide, a cell, or an organism, refers to a nucleic acid, polypeptide, cell, or organism that is found in nature. For example, a polypeptide or polynucleotide sequence that is present in an organism (including viruses) that can be isolated from a source in nature and which has not been intentionally modified by a human in the laboratory is wild type (and naturally occurring).

“Recombinant,” as used herein, means that a particular nucleic acid (DNA or RNA) is the product of various combinations of cloning, including but not limited to restriction, polymerase chain reaction (PCR) and/or ligation steps resulting in a construct having a structural coding or non-coding sequence distinguishable from endogenous nucleic acids found in natural systems. DNA sequences encoding polypeptides can be assembled from cDNA fragments or from a series of synthetic oligonucleotides, to provide a synthetic nucleic acid which is capable of being expressed from a recombinant transcriptional unit contained in a cell or in a cell-free transcription and translation system. Genomic DNA comprising the relevant sequences can also be used in the formation of a recombinant gene or transcriptional unit. Sequences of non-translated DNA may be present 5′ or 3′ from the open reading frame, where such sequences do not interfere with manipulation or expression of the coding regions, and may indeed act to modulate production of a desired product by various mechanisms (see “DNA regulatory sequences”, below). Alternatively, DNA sequences encoding RNA (e.g., Cas13 guide RNA) that is not translated may also be considered recombinant. Thus, e.g., the term “recombinant” nucleic acid refers to one which is not naturally occurring, e.g., is made by the artificial combination of two otherwise separated segments of sequence through human intervention. This artificial combination is often accomplished by either chemical synthesis means, or by the artificial manipulation of isolated segments of nucleic acids, e.g., by genetic engineering techniques. Such is usually done to replace a codon with a codon encoding the same amino acid, a conservative amino acid, or a non-conservative amino acid. Alternatively, it is performed to join together nucleic acid segments of desired functions to generate a desired combination of functions. This artificial combination is often accomplished by either chemical synthesis means, or by the artificial manipulation of isolated segments of nucleic acids, e.g., by genetic engineering techniques. Recombinant polypeptides or nucleic acids may also be synthesized using methods known in the art. When a recombinant polynucleotide encodes a polypeptide, the sequence of the encoded polypeptide can be naturally occurring (“wild type”) or can be a variant (e.g., a mutant) of the naturally occurring sequence. Thus, the term “recombinant” polypeptide does not necessarily refer to a polypeptide whose sequence does not naturally occur. Instead, a “recombinant” polypeptide is encoded by a recombinant DNA sequence, but the sequence of the polypeptide can be naturally occurring (“wild type”) or non-naturally occurring (e.g., a variant, a mutant, etc.). Thus, a “recombinant” polypeptide is the result of human intervention, but may be a naturally occurring amino acid sequence.

A “vector” or “expression vector” is a replicon, such as plasmid, phage, virus, or cosmid, to which another DNA segment, i.e. an “insert”, may be attached so as to bring about the replication of the attached segment in a cell.

As used herein cis-nuclease activity refers to nuclease activity directed towards a nucleic acid target to which a CRISPR guide RNA is hybridized. Trans-nuclease activity refers to nuclease activity directed towards a nucleic acid target to which a CRISPR guide RNA is not-hybridized

An “expression cassette” comprises a DNA coding sequence operably linked to a promoter. “Operably linked” refers to a juxtaposition wherein the components so described are in a relationship permitting them to function in their intended manner. For instance, a promoter is operably linked to a coding sequence if the promoter affects its transcription or expression.

The terms “recombinant expression vector,” or “DNA construct” are used interchangeably herein to refer to a DNA molecule comprising a vector and one insert. Recombinant expression vectors are usually generated for the purpose of expressing and/or propagating the insert(s), or for the construction of other recombinant nucleotide sequences. The insert(s) may or may not be operably linked to a promoter sequence and may or may not be operably linked to DNA regulatory sequences.

Target specificity can be used in reference to a guide RNA, or a crRNA specific to a target polynucleotide sequence or region and further includes a sequence of nucleotides capable of selectively annealing/hybridizing to a target (sequence or region) of a target polynucleotide, e.g., a target DNA. Target specific nucleotides can have a single species of oligonucleotide, or it can include two or more species with different sequences. Thus, the target specific nucleotide can be two or more sequences, including 3, 4, 5, 6, 7, 8, 9 or 10 or more different sequences. In certain embodiments, a crRNA or the derivative thereof contains a target-specific nucleotide region complementary to a region of the target DNA sequence. In certain embodiments, a crRNA or the derivative thereof may contain other nucleotide sequences besides a target-specific nucleotide region. In certain embodiments, the other nucleotide sequences may be from a tracrRNA sequence.

Hybridization, as used herein, generally refers to and includes the capacity and/or ability of a first nucleic acid molecule to non-covalently bind (e.g., form Watson-Crick-base pairs and/or G/U base pairs), anneal, and/or hybridize to a second nucleic acid molecule under the appropriate or certain in vitro and/or in vivo conditions of temperature, pH, and/or solution ionic strength. Generally, standard Watson-Crick base pairing includes; adenine (A) pairing with thymidine (T); adenine (A) pairing with uracil (U); and guanine (G) pairing with cytosine (C). In some embodiments, hybridization comprises at least two nucleic acids comprising complementary sequences (e.g., fully complementary, substantially complementary, or partially complementary). In certain embodiments, hybridization comprises at least two nucleic acids comprising fully complementary sequences.

Any given component, or combination of components can be unlabeled, or can be detectably labeled with a label moiety. The detectable label may be a fluorescent, luminescent, phosphorescent, magnetic or radioactive label. In some cases, when two or more components are labeled, they can be labeled with label moieties that are distinguishable from one another.

Nucleases can have cleavage activity both on-target (cis) and off-target (trans), and variants that that modulate the activity of cis and/or trans activity find many useful applications, from improving the sensitivity of viral diagnostics to providing a robust scaffold for RNA editors.

Cell-based approaches to enzyme screening come with inherent limitations such as non-specific interactions between cellular components, restricted throughput, and complex transfection protocols.

### Methods

Described herein are methods of generating variant nucleases with altered function in nuclease target sequence binding and/and or cutting.

Cell-free methods may be superior to cell-based methods because it generally has an advantage in speed and scalability. Cell-free transcription-translation (TXTL) systems can measure the DNA or RNA cleavage dynamics of CRISPR effectors to identify or validate Cas13 variant and orthologs having altered binding dynamics or catalytic activity. Cell-free TXTL then can be a suitable platform for screening Cas13 variants for altered nuclease activity.

Double emulsion (DE) droplets have advantages in compartmentalizing enzyme evolution and single-molecule analysis. A platform to generate monodisperse, FACS-compatible DE droplets is particularly advantageous for establishing direct links between sequence and phenotype. Each water/oil/water droplet contains a single variant of interest and can accommodate a range of biologically relevant reagents. Unlike traditional single-emulsions, each DE droplet can undergo both fluorescence-based phenotyping and downstream sequencing, circumventing the expense of fluorescence activated droplet sorting (FADS) and the non-specific nature of pooled sequencing. Prior work has demonstrated that DE droplets can be clustered by fluorescence through FACS, support RT-qPCR, encapsulate mammalian cells, and sort variants with single-droplet precision. The present disclosure provides methods amounting to the field's first large-scale screen of Cas13 enzyme orthologs.

The methods described herein comprise a series of compartmentalized reactions and can be visualized by the methods referred to in FIG. 1. The first compartmentalized reaction comprises one or more reagents to amplify, and optionally purify a variant nuclease template nucleic acid. The second compartmentalized reaction comprises one or more reagents to synthesize a variant nuclease polypeptide by transcribing and translating the nucleic acid encoding the variant nuclease. The third compartmentalized reaction comprises one or more assays to screen the nuclease activity of said variant polypeptide. Screening assays may include but are not limited to FACS sorting, fluorescence quenching, comparative cis and trans nuclease measurements, and validation by SHINE or SHERLOCK assay.

SHERLOCK (Specific High Sensitivity Enzymatic Reporter unLOCKing) is a CRISPR-based diagnostic platform for specific recognition of desired DNA or RNA sequences. SHERLOCK detects RNA or DNA using nucleic acid pre-amplification with CRISPR-Cas enzymology as generally described in Kellner, et al. “SHERLOCK: Nucleic acid detection with CRISPR nucleases,” Nat Protoc. October; 14 (10): 2986-3012 (2019).

SHINE (SHERLOCK and HUDSON Integration to Navigate Epidemics) is a diagnostic method for RNA detection from unextracted samples. SHINE comprises optimizing and combining SHERLOCK into a single-step reaction where SHINE's results can be visualized with an in-tube fluorescent readout. SHINE is generally described in Arizti-Sanz, et al. in “Streamlined inactivation, amplification, and Cas13-based detection of SARS-COV-2.” Nat Commun 11, 5921 (2020).

### Methods of Engineering Nucleases

The following method is generally applicable to all Cas nucleases or nucleases that possess nuclease activity.

CRISPR-Cas nucleases edit a genome efficiently in a wide variety of organisms and cell types (Sander et al., “CRISPR-Cas systems for editing, regulating and targeting genomes” Nature Biotechnology, 32, 347-355 (2014). As described herein, Cas proteins can be engineered to show altered specificity in binding dynamics or catalytic activity.

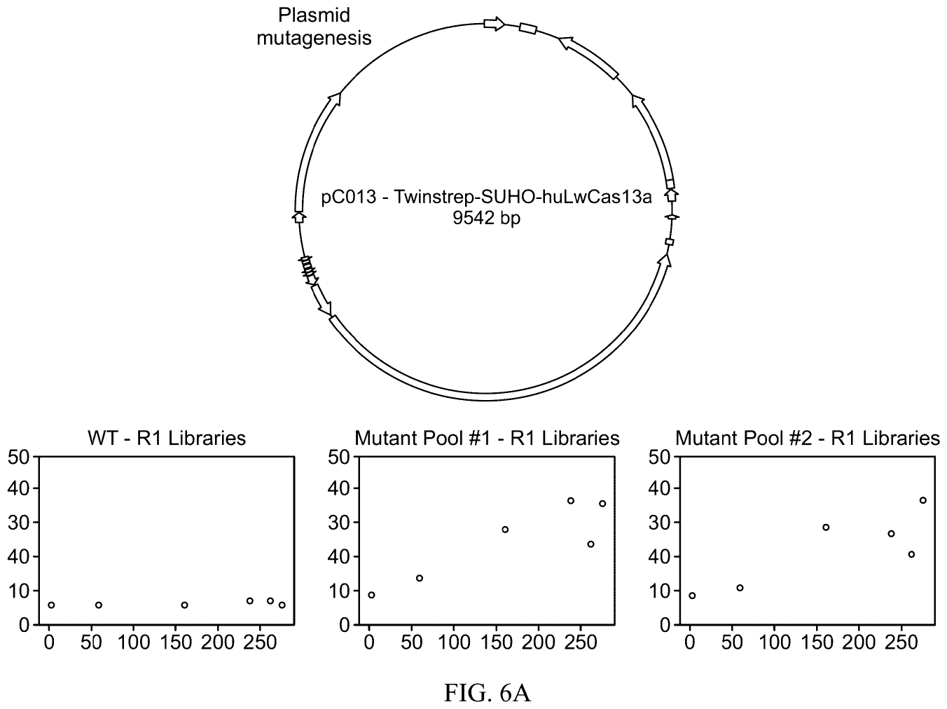

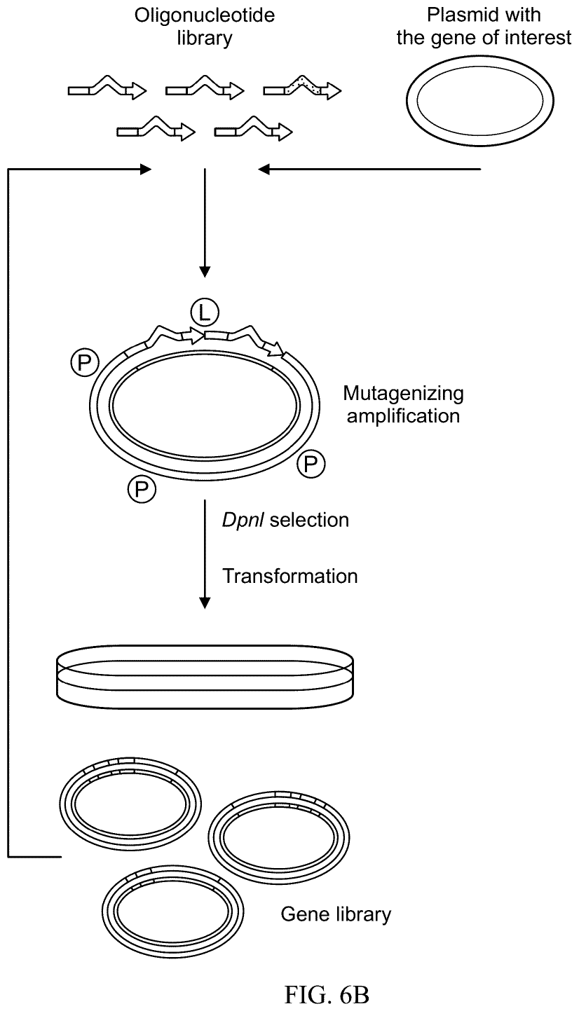

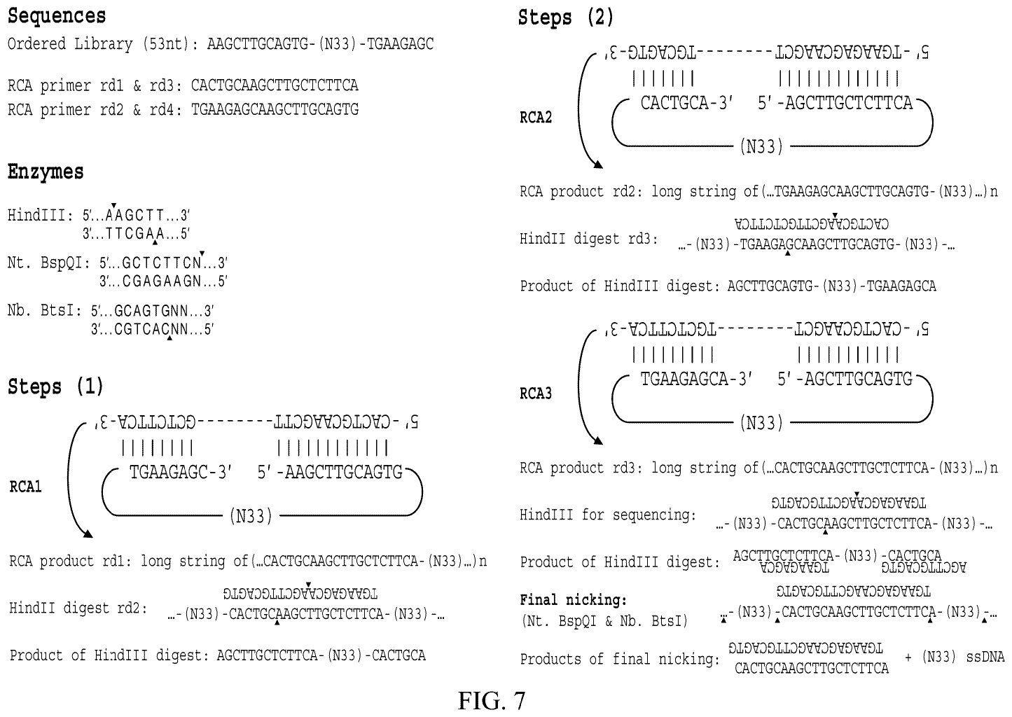

A library of Cas nuclease variants may be designed through targeted mutagenesis of specific domains. For example, as shown in FIGS. 6 and 7, a library of Cas nuclease variants may be designed using an oligonucleotide library for mutagenesis of plasmids containing a nucleic acid encoding a Cas protein. An oligonucleotide library can be created using rolling circle amplification (RCA). See, for example, Schmidt et al., Scalable amplification of strand subsets from chip-synthesized oligonucleotide libraries, 2015; 6: 8634.

As with other Class 2 CRISPR enzymes, Cas13 protein has a bi-lobed architecture, containing a nuclease (NUC) lobe and a crRNA recognition (REC) lobe. Two higher eukaryotes and prokaryotes nucleotide (HEPN)-binding domains in the NUC lobe are responsible for the complex's RNase activity.

Cas13 is an RNA-targeting CRISPR enzyme that exhibits both on-target (cis) and off-target (trans) cleavage activity. Prior screens of Cas9 and Cas12 have yielded variants with enhanced specificity, compact size, and broader targeting range. A high-throughput screen of Cas13 variants therefore promises to expand the enzyme's uses in RNA-oriented applications.

Specifically, site-directed mutagenesis may be targeted to one or more domains selected from the list consisting of a HEPN-1 domain, a HEPN-2 domain, a Helical-1 domain, a Helical-2 domain, a Cas switch region, a Cas nuclease lobe, a Cas recognition lobe, a Cas linker region, a NTD, a Monolith region, a Cas catalytic site, and combinations thereof. Alternatively, a ready-to-clone DNA library can be obtained from a commercial source or can be produced using techniques known to the skilled artisan. Cas nucleases may also be engineered to contain a unique barcode tag and a universal ‘handle’ appended to each library variant for downstream identification of the nuclease sequence. Each variant-barcode association may be determined by deep sequencing. The DNA library may also be cloned into a custom vector or plasmid backbone, transformed into competent E. coli cells, and extracted via plasmid purification.

In another aspect, a method for engineering Cas12 or Cas13 nucleases comprises low-throughput screening of mammalian cells to determine Cas nuclease cis and/or trans activity with respect to targeted exogenous or endogenous genes. For example, mammalian cells can be cotransfected with a plasmid coding for a Cas12 nuclease or Cas13 nuclease and a first fluorescent reporter protein, and a plasmid coding for a guide RNA (gRNA) and a second fluorescent reporter protein. Alternatively, a dual reporter system, such as a dual-fluorescence reporter system can be used to target endogenous genes in mammalian cells to determine Cas nuclease activity. In some aspects, the mammalian cell can be a HEK293 cell or CHO cell.

In other aspects, the Cas nucleases are engineered using rational design such as, for example, insertion of RNA binding domains into a nucleotide-binding domain.

A library of variant Cas nucleases may be generated using a nuclease selected from the list consisting of a, Cas12a, Cas12b, Cas12d, Cas13a, a Cas13b, a Cas13d, or a CasRx. A library of variant Cas13 nucleases may be generated using random or site-directed mutagenesis for Leptotrichia wadei Cas13a (LwaCas13a) Leptotrichia buccalis Cas13a (LbuCas13a), Ruminococcus flavefaciens XPD3002 (RfxCas13d (CasRx)), or Prevotella sp. P5-125 (PspCas13b) nucleases.

Each variant may differ from a wild-type sequence by 1, 2, 3, 4, 5, 6, 7, 8, 9, or 10 or more amino acid substitutions, insertions, or deletions.

A library of variant Cas13 nucleases may be generated using a nuclease selected from a nuclease with at least about 85%, 90%, 95%, 97%, 98% %, 99%, or 100% identity to SEQ ID NO: 1. Each variant may differ from SEQ ID NO: 1 by 1, 2, 3, 4, 5, 6, 7, 8, 9, or 10 or more amino acid substitutions, insertions, or deletions.

A library of variant Cas13 nucleases may be generated using a nuclease encoded by a nucleic acid possessing at least 85%, 90%, 95%, 97%, 98%, 99%, or 100% homology to SEQ ID NO: 2.

A library of variant Cas13 nucleases may be generated using a nuclease selected from a nuclease with at least about 85%, 90%, 95%, 97%, 98% %, 99%, or 100% identity to SEQ ID NO: 3. Each variant may differ from SEQ ID NO: 3 by 1, 2, 3, 4, 5, 6, 7, 8, 9, or 10 or more amino acid substitutions, insertions, or deletions.

A library of variant Cas13 nucleases may be generated using a nuclease encoded by a nucleic acid possessing at least 85%, 90%, 95%, 97%, 98%, 99%, or 100% homology to SEQ ID NO: 4.

A library of variant Cas13 nucleases may be generated using a nuclease selected from a nuclease with at least about 85%, 90%, 95%, 97%, 98% %, 99%, or 100% identity to SEQ ID NO: 5. Each variant may differ from SEQ ID NO: 5 by 1, 2, 3, 4, 5, 6, 7, 8, 9, or 10 or more amino acid substitutions, insertions, or deletions.

A library of variant Cas13 nucleases may be generated using a nuclease encoded by a nucleic acid possessing at least 85%, 90%, 95%, 97%, 98%, 99%, or 100% homology to SEQ ID NO: 6.

A library of variant Cas13 nucleases may be generated using a nuclease selected from a nuclease with at least about 85%, 90%, 95%, 97%, 98% %, 99%, or 100% identity to SEQ ID NO: 7. Each variant may differ from SEQ ID NO: 7 by 1, 2, 3, 4, 5, 6, 7, 8, 9, or 10 or more amino acid substitutions, insertions, or deletions.

A library of variant Cas13 nucleases may be generated using a nuclease encoded by a nucleic acid possessing at least 85%, 90%, 95%, 97%, 98%, 99%, or 100% homology to SEQ ID NO: 8.

The methods described herein are capable of screening a large amount of variant nucleic acids in a single assay. The methods described herein may screen at least about 10 variant nucleic acids in a single assay to about 20,000 variant nucleic acids in a single assay. The methods described herein may screen at least about 10 variant nucleic acids in a single assay to about 50 variant nucleic acids in a single assay, about 10 variant nucleic acids in a single assay to about 100 variant nucleic acids in a single assay, about 10 variant nucleic acids in a single assay to about 500 variant nucleic acids in a single assay, about 10 variant nucleic acids in a single assay to about 1,000 variant nucleic acids in a single assay, about 10 variant nucleic acids in a single assay to about 2,000 variant nucleic acids in a single assay, about 10 variant nucleic acids in a single assay to about 5,000 variant nucleic acids in a single assay, about 10 variant nucleic acids in a single assay to about 10,000 variant nucleic acids in a single assay, about 10 variant nucleic acids in a single assay to about 20,000 variant nucleic acids in a single assay, about 10 variant nucleic acids in a single assay to about 100,000 variant nucleic acids in a single assay, about 50 variant nucleic acids in a single assay to about 100 variant nucleic acids in a single assay, about 50 variant nucleic acids in a single assay to about 500 variant nucleic acids in a single assay, about 50 variant nucleic acids in a single assay to about 1,000 variant nucleic acids in a single assay, about 50 variant nucleic acids in a single assay to about 2,000 variant nucleic acids in a single assay, about 50 variant nucleic acids in a single assay to about 5,000 variant nucleic acids in a single assay, about 50 variant nucleic acids in a single assay to about 10,000 variant nucleic acids in a single assay, about 50 variant nucleic acids in a single assay to about 20,000 variant nucleic acids in a single assay, about 50 variant nucleic acids in a single assay to about 100,000 variant nucleic acids in a single assay, about 100 variant nucleic acids in a single assay to about 500 variant nucleic acids in a single assay, about 100 variant nucleic acids in a single assay to about 1,000 variant nucleic acids in a single assay, about 100 variant nucleic acids in a single assay to about 2,000 variant nucleic acids in a single assay, about 100 variant nucleic acids in a single assay to about 5,000 variant nucleic acids in a single assay, about 100 variant nucleic acids in a single assay to about 10,000 variant nucleic acids in a single assay, about 100 variant nucleic acids in a single assay to about 20,000 variant nucleic acids in a single assay, about 100 variant nucleic acids in a single assay to about 100,000 variant nucleic acids in a single assay, about 500 variant nucleic acids in a single assay to about 1,000 variant nucleic acids in a single assay, about 500 variant nucleic acids in a single assay to about 2,000 variant nucleic acids in a single assay, about 500 variant nucleic acids in a single assay to about 5,000 variant nucleic acids in a single assay, about 500 variant nucleic acids in a single assay to about 10,000 variant nucleic acids in a single assay, about 500 variant nucleic acids in a single assay to about 20,000 variant nucleic acids in a single assay, about 500 variant nucleic acids in a single assay to about 100,000 variant nucleic acids in a single assay, about 1,000 variant nucleic acids in a single assay to about 2,000 variant nucleic acids in a single assay, about 1,000 variant nucleic acids in a single assay to about 5,000 variant nucleic acids in a single assay, about 1,000 variant nucleic acids in a single assay to about 10,000 variant nucleic acids in a single assay, about 1,000 variant nucleic acids in a single assay to about 20,000 variant nucleic acids in a single assay, about 1,000 variant nucleic acids in a single assay to about 100,000 variant nucleic acids in a single assay, about 2,000 variant nucleic acids in a single assay to about 5,000 variant nucleic acids in a single assay, about 2,000 variant nucleic acids in a single assay to about 10,000 variant nucleic acids in a single assay, about 2,000 variant nucleic acids in a single assay to about 20,000 variant nucleic acids in a single assay, about 2,000 variant nucleic acids in a single assay to about 100,000 variant nucleic acids in a single assay, about 5,000 variant nucleic acids in a single assay to about 10,000 variant nucleic acids in a single assay, about 5,000 variant nucleic acids in a single assay to about 20,000 variant nucleic acids in a single assay, about 5,000 variant nucleic acids in a single assay to about 100,000 variant nucleic acids in a single assay, about 10,000 variant nucleic acids in a single assay to about 20,000 variant nucleic acids in a single assay, about 10,000 variant nucleic acids in a single assay to about 100,000 variant nucleic acids in a single assay, or about 20,000 variant nucleic acids in a single assay to about 100,000 variant nucleic acids in a single assay. The methods described herein may screen at least about 10 variant nucleic acids in a single assay, about 50 variant nucleic acids in a single assay, about 100 variant nucleic acids in a single assay, about 500 variant nucleic acids in a single assay, about 1,000 variant nucleic acids in a single assay, about 2,000 variant nucleic acids in a single assay, about 5,000 variant nucleic acids in a single assay, about 10,000 variant nucleic acids in a single assay, about 20,000 variant nucleic acids in a single assay, or about 100,000 variant nucleic acids in a single assay. The methods described herein may screen at least at least about 10 variant nucleic acids in a single assay, about 50 variant nucleic acids in a single assay, about 100 variant nucleic acids in a single assay, about 500 variant nucleic acids in a single assay, about 1,000 variant nucleic acids in a single assay, about 2,000 variant nucleic acids in a single assay, about 5,000 variant nucleic acids in a single assay, about 10,000 variant nucleic acids in a single assay or about 20,000 variant nucleic acids in a single assay. The methods described herein may screen at least at most about 50 variant nucleic acids in a single assay, about 100 variant nucleic acids in a single assay, about 500 variant nucleic acids in a single assay, about 1,000 variant nucleic acids in a single assay, about 2,000 variant nucleic acids in a single assay, about 5,000 variant nucleic acids in a single assay, about 10,000 variant nucleic acids in a single assay, about 20,000 variant nucleic acids in a single assay, or about 100,000 variant nucleic acids in a single assay.

A nucleic acid encoding the variant nucleases herein can comprise a unique barcode to aid downstream identification of nucleases that exhibit desirable nuclease properties. Unique barcode tags appended to a nucleic acid encoding a nuclease may contain about 10 base-pairs to about 50 base-pairs. Unique barcode tags may contain about 10 base-pairs to about 20 base-pairs, about 10 base-pairs to about 30 base-pairs, about 10 base-pairs to about 40 base-pairs, about 10 base-pairs to about 50 base-pairs, about 20 base-pairs to about 30 base-pairs, about 20 base-pairs to about 40 base-pairs, about 20 base-pairs to about 50 base-pairs, about 30 base-pairs to about 40 base-pairs, about 30 base-pairs to about 50 base-pairs, or about 40 base-pairs to about 50 base-pairs. Unique barcode tags may contain about 10 base-pairs, about 20 base-pairs, about 30) base-pairs, about 40 base-pairs, or about 50) base-pairs. Unique barcode tags may contain at least about 10 base-pairs, about 20 base-pairs, about 30 base-pairs, or about 40) base-pairs. Unique barcode tags may contain at most about 20 base-pairs, about 30 base-pairs, about 40) base-pairs, or about 50) base-pairs.

A nucleic acid encoding the variant nucleases herein can comprise a universal handle sequence or universal primer binding site to facilitate nucleic acid sequencing. A universal ‘handle’ sequence appended to a nucleic acid encoding a nuclease may contain about 5 base-pairs to about 30 base-pairs. A universal ‘handle’ sequence appended to a nuclease may contain about 5 base-pairs to about 10 base-pairs, about 5 base-pairs to about 15 base-pairs, about 5 base-pairs to about 20 base-pairs, about 5 base-pairs to about 30 base-pairs, about 10) base-pairs to about 15 base-pairs, about 10 base-pairs to about 20 base-pairs, about 10 base-pairs to about 30 base-pairs, about 15 base-pairs to about 20 base-pairs, about 15 base-pairs to about 30 base-pairs, or about 20 base-pairs to about 30 base-pairs. A universal ‘handle’ sequence appended to a nuclease may contain about 5 base-pairs, about 10 base-pairs, about 15 base-pairs, about 20 base-pairs, or about 30 base-pairs. A universal ‘handle’ sequence appended to a nuclease may contain at least about 5 base-pairs, about 10 base-pairs, about 15 base-pairs, or about 20 base-pairs. A universal ‘handle’ sequence appended to a nuclease may contain at most about 10 base-pairs, about 15 base-pairs, about 20 base-pairs, or about 30 base-pairs.

### Compartmentalized Reactions

In a certain embodiment of the methods described herein, the compartmentalized reactions may amplify, modify, isolate, or purify a target polynucleotide in a reaction vessel. A compartmentalized reaction may also contain one or more of the following: transcribing a template nucleotide sequence to RNA; amplifying, modifying, isolating, or purifying said RNA in said reaction vessel; translating a RNA to DNA, LNA, oligonucleotide, or other nucleotide sequence; amplifying, modifying, isolating, or purifying said nucleotide sequence in said reaction vessel; and translating a RNA or other nucleotide sequence to polypeptide sequence.

Proteins can be expressed in a compartmentalized reaction using biomolecular translation machinery extracted from cells (“cell lysates”). Cell-free protein production can be accomplished with several kinds and species of cell extract, and cell-free proteins can also be generated by purified buffers, proteins, nucleotides, and template nucleotide.

A reaction chamber may include but is not limited to a well of dish, multiwell plate, or a hydrophilic compartment of an inverse emulsion.

In a certain embodiment of the methods described herein the compartmentalized reactions may be performed in an emulsion. While a cell membrane constitutes an amphiphilic interface between the interior of the cell and its environment, this barrier can be mimicked by encapsulating transcription-translation reactions inside double (water-in-oil-in-water) emulsions in order to study large number of these reactions at a cellular scale. Large numbers of TXTL microdroplets in oil with a controlled disparity can be generated, stored, and remain stable for screening.

Methods of generating double emulsion (DE) droplets include but are not limited to using a microfluidic device or phase-separated polymer solution. Microfluidic devices for generating DE compartmentalized reactions for example are described generally by Macosko, et al. in “Highly parallel genome-wide expression profiling of individual cells using nanoliter droplets” Cell, 161 (5), 1202-1214 (2015). Microfluidic devices for generating DE compartmentalized reactions for Mastrobattista, et al. in “High-throughput screening of enzyme libraries; in vitro evolution of a β-galactosidase by fluorescence-activated sorting of double emulsions” Chemistry & Biology, 12 (12), 1291-1300 (2005). Said microfluidic method may comprise aqueous solution, an oil solution, a syringe or vacuum pump, and a microfluidic chip to generate an aqueous volume containing individual oil barriers, wherein said oil barriers surrounded another aqueous solution for individual reactions.

Phase separated polymer solution may also generate DE droplets, for example as described generally in Torre, et al. “Multiphase Water-in-Oil Emulsion Droplets for Cell-Free Transcription-Translation” Langmuir. 30 (20): 5695-5699 (2014).

DE droplets are advantageous for establishing direct links between sequence and phenotype. Each DE droplet is characterized by a water/oil/water layer, contains a single variant of interest, and can accommodate a range of biologically relevant reagents. DE droplets can be created through a droplet microfluidics platform with the advantages of being monodisperse and FACS-compatible. Unlike traditional single-emulsions, DE droplet each can undergo both fluorescence-based phenotyping and downstream sequencing, circumventing the expense of fluorescence activated droplet sorting (FADS) and the non-specific nature of pooled sequencing. Another advantage of DE droplets is that DE droplets can be clustered by fluorescence through FACS, support RT-qPCR, encapsulate mammalian cells, and sort variants with single-droplet precision.

The emulsion for use with the methods described herein may comprise 100 droplets to 10,000 droplets per microliter. The emulsion for use with the methods described herein may comprise 100 droplets to 200 droplets, 100 droplets to 500 droplets, 100 droplets to 1,000 droplets, 100 droplets to 2,000 droplets, 100 droplets to 5,000 droplets, 100 droplets to 10,000 droplets, 200 droplets to 500 droplets, 200 droplets to 1,000 droplets, 200 droplets to 2,000 droplets, 200 droplets to 5,000 droplets, 200 droplets to 10,000 droplets, 500 droplets to 1,000 droplets, 500 droplets to 2,000 droplets, 500 droplets to 5,000 droplets, 500 droplets to 10,000 droplets, 1,000 droplets to 2,000 droplets, 1,000 droplets to 5,000 droplets, 1,000 droplets to 10,000 droplets, 2,000 droplets to 5,000 droplets, 2,000 droplets to 10,000 droplets, or 5,000 droplets to 10,000 droplets per microliter. The emulsion for use with the methods described herein may comprise 100 droplets, 200 droplets, 500 droplets, 1,000 droplets, 2,000 droplets, 5,000 droplets, or 10,000 droplets per microliter. The emulsion for use with the methods described herein may comprise at least 100 droplets, 200 droplets, 500 droplets, 1,000 droplets, 2,000 droplets, or 5,000 droplets per microliter. The emulsion for use with the methods described herein may comprise at most 200 droplets, 500 droplets, 1,000 droplets, 2,000 droplets, 5,000 droplets, or 10,000 droplets per microliter.

A droplet may comprise a volume of at least about 1 picoliter to about 1,000 picoliters. A droplet may comprise a volume of at least about 1 picoliter to about 2 picoliters, about 1 picoliter to about 5 picoliters, about 1 picoliter to about 10 picoliters, about 1 picoliter to about 25 picoliters, about 1 picoliter to about 50 picoliters, about 1 picoliter to about 100 picoliters, about 1 picoliter to about 200 picoliters, about 1 picoliter to about 500 picoliters, about 1 picoliter to about 1,000 picoliters, about 2 picoliters to about 5 picoliters, about 2 picoliters to about 10 picoliters, about 2 picoliters to about 25 picoliters, about 2 picoliters to about 50) picoliters, about 2 picoliters to about 100 picoliters, about 2 picoliters to about 200 picoliters, about 2 picoliters to about 500 picoliters, about 2 picoliters to about 1,000 picoliters, about 5 picoliters to about 10 picoliters, about 5 picoliters to about 25 picoliters, about 5 picoliters to about 50 picoliters, about 5 picoliters to about 100 picoliters, about 5 picoliters to about 200 picoliters, about 5 picoliters to about 500 picoliters, about 5 picoliters to about 1,000 picoliters, about 10 picoliters to about 25 picoliters, about 10 picoliters to about 50 picoliters, about 10 picoliters to about 100 picoliters, about 10 picoliters to about 200 picoliters, about 10 picoliters to about 500 picoliters, about 10 picoliters to about 1,000 picoliters, about 25 picoliters to about 50 picoliters, about 25 picoliters to about 100 picoliters, about 25 picoliters to about 200 picoliters, about 25 picoliters to about 500 picoliters, about 25 picoliters to about 1,000 picoliters, about 50 picoliters to about 100 picoliters, about 50 picoliters to about 200 picoliters, about 50 picoliters to about 500 picoliters, about 50 picoliters to about 1,000 picoliters, about 100 picoliters to about 200 picoliters, about 100 picoliters to about 500 picoliters, about 100 picoliters to about 1,000 picoliters, about 200 picoliters to about 500 picoliters, about 200 picoliters to about 1,000 picoliters, or about 500 picoliters to about 1,000 picoliters. A droplet may comprise a volume of at least about 1 picoliter, about 2 picoliters, about 5 picoliters, about 10 picoliters, about 25 picoliters, about 50 picoliters, about 100 picoliters, about 200 picoliters, about 500 picoliters, or about 1,000 picoliters. A droplet may comprise a volume of at least at least about 1 picoliter, about 2 picoliters, about 5 picoliters, about 10 picoliters, about 25 picoliters, about 50 picoliters, about 100 picoliters, about 200 picoliters, or about 500 picoliters. A droplet may comprise a volume of at least at most about 2 picoliters, about 5 picoliters, about 10 picoliters, about 25 picoliters, about 50 picoliters, about 100 picoliters, about 200 picoliters, about 500 picoliters, or about 1,000 picoliters.

In some embodiments, carrier molecules can be beads composed of or coated with magnetic material, glass, polyacrylamide, polystyrene, protein A, protein G, streptavidin, antibodies, and silanized material. In other embodiments, a magnetic bead may be coated with protein A, protein G, streptavidin, antibodies or silanized material.

In some embodiments, carrier molecules can be complexed to a variant template nucleic acid. A carrier molecule complex may comprise a variant Cas polypeptide, a guide RNA, and a bead to form a Cas-guide RNA-bead complex. A carrier molecule complex may comprise a variant Cas polypeptide, a variant crRNA, and a bead to a Cas-crRNA-bead complex. Said variant crRNA may comprise of a barcode region to identify the variant template nucleic acid or to complex said bead with a crRNA.

The first compartmentalized reaction is referred to FIG. 2, which comprises one or more reagents including but not limited to dNTPs, PCR buffer, PCR polymerase, variant nuclease primers, variant nuclease template DNA, and a bead for purification of variant RNA. Said first compartmentalized reaction generates variant RNA from said variant nuclease template DNA, which can be generated through PCR or isothermal amplification. FIG. 2 can alternatively comprise one or more reagents for isothermal amplification instead of PCR. Each droplet may also contain a bead and Cas plasmid, wherein crRNA of a variant nuclease sequence is complexed to a barcode sequence, and said barcode sequence is captured or complexed to a bead.

A second compartmentalized reaction is referred to FIG. 3 and comprises one or more reagents including but not limited to a bead for purification of variant RNA, transcriptase polypeptide, buffer for DNA transcription, dNTPs, and oligonucleotide primers with complementary sequences to said variant RNA.

A second compartmentalized reaction can generate variant nuclease polypeptides in a cell-free emulsion. A variant nucleic acid sequence from a first emulsion may comprise a Cas RNA-barcode-bead complex, and said Cas RNA-barcode-bead complex may then be translated and transcribed to generate variant Cas polypeptide. In one aspect, said variant Cas polypeptide may bind to crRNA and form a Cas-crRNA-barcode-bead complex. In one embodiment, said Cas-crRNA-barcode-bead complex can be purified from a second emulsion using said bead.

Synthesis of naturally-occurring or variant polypeptides is essential for biomedical research, diagnostics, and therapeutics. Polypeptide synthesis may be performed within the environment of a cell, or using cellular extracts and coding sequences to synthesize proteins in vitro. In vitro polypeptide synthesis reactions in a cell-free environment include but are not limited to amplifying a targeted polynucleotide sequence, wherein said targeted polynucleotide sequence is present in a complex mixture of sequences with a 5′ and a 3′ primer; transcribing the amplification product into mRNA with a RNA polymerase; and translating said mRNA product into DNA with a RNA polymerase. RNA and DNA mimetics may also be substituted, for example but not limited to LNA as a substitution for RNA or DNA.

An in vitro transcription and translation reaction can generate about 1 polypeptide to about 100,000 polypeptides. An in vitro transcription and translation reaction can generate about 1 polypeptide to about 10 polypeptides, about 1 polypeptide to about 50 polypeptides, about 1 polypeptide to about 100 polypeptides, about 1 polypeptide to about 500 polypeptides, about 1 polypeptide to about 1,000 polypeptides, about 1 polypeptide to about 2,000 polypeptides, about 1 polypeptide to about 5,000 polypeptides, about 1 polypeptide to about 10,000 polypeptides, about 1 polypeptide to about 20,000 polypeptides, about 1 polypeptide to about 50,000 polypeptides, about 1 polypeptide to about 100,000 polypeptides, about 10 polypeptides to about 50 polypeptides, about 10 polypeptides to about 100 polypeptides, about 10 polypeptides to about 500 polypeptides, about 10 polypeptides to about 1,000 polypeptides, about 10 polypeptides to about 2,000 polypeptides, about 10 polypeptides to about 5,000 polypeptides, about 10 polypeptides to about 10,000 polypeptides, about 10 polypeptides to about 20,000 polypeptides, about 10 polypeptides to about 50,000 polypeptides, about 10 polypeptides to about 100,000 polypeptides, about 50 polypeptides to about 100 polypeptides, about 50 polypeptides to about 500 polypeptides, about 50 polypeptides to about 1,000 polypeptides, about 50 polypeptides to about 2,000 polypeptides, about 50 polypeptides to about 5,000 polypeptides, about 50 polypeptides to about 10,000 polypeptides, about 50 polypeptides to about 20,000 polypeptides, about 50 polypeptides to about 50,000 polypeptides, about 50 polypeptides to about 100,000 polypeptides, about 100 polypeptides to about 500 polypeptides, about 100 polypeptides to about 1,000 polypeptides, about 100 polypeptides to about 2.000 polypeptides, about 100 polypeptides to about 5,000 polypeptides, about 100 polypeptides to about 10,000 polypeptides, about 100 polypeptides to about 20,000 polypeptides, about 100 polypeptides to about 50,000 polypeptides, about 100 polypeptides to about 100,000 polypeptides, about 500 polypeptides to about 1,000 polypeptides, about 500 polypeptides to about 2,000 polypeptides, about 500 polypeptides to about 5,000 polypeptides, about 500 polypeptides to about 10,000 polypeptides, about 500 polypeptides to about 20,000 polypeptides, about 500 polypeptides to about 50,000 polypeptides, about 500 polypeptides to about 100,000 polypeptides, about 1,000 polypeptides to about 2,000 polypeptides, about 1,000 polypeptides to about 5,000 polypeptides, about 1,000 polypeptides to about 10,000 polypeptides, about 1,000 polypeptides to about 20,000 polypeptides, about 1,000 polypeptides to about 50,000 polypeptides, about 1,000 polypeptides to about 100,000 polypeptides, about 2,000 polypeptides to about 5,000 polypeptides, about 2,000 polypeptides to about 10,000 polypeptides, about 2,000 polypeptides to about 20,000 polypeptides, about 2,000 polypeptides to about 50,000 polypeptides, about 2,000 polypeptides to about 100,000 polypeptides, about 5,000 polypeptides to about 10,000 polypeptides, about 5,000 polypeptides to about 20,000 polypeptides, about 5,000 polypeptides to about 50,000 polypeptides, about 5,000 polypeptides to about 100,000 polypeptides, about 10,000 polypeptides to about 20,000 polypeptides, about 10,000 polypeptides to about 50,000 polypeptides, about 10,000 polypeptides to about 100,000 polypeptides, about 20,000 polypeptides to about 50,000 polypeptides, about 20,000 polypeptides to about 100,000 polypeptides, or about 50,000 polypeptides to about 100,000 polypeptides. An in vitro transcription and translation reaction can generate about 1 polypeptide, about 10 polypeptides, about 50 polypeptides, about 100 polypeptides, about 500 polypeptides, about 1,000 polypeptides, about 2,000 polypeptides, about 5,000 polypeptides, about 10,000 polypeptides, about 20,000 polypeptides, about 50,000 polypeptides, or about 100,000 polypeptides. An in vitro transcription and translation reaction can generate at least about 1 polypeptide, about 10 polypeptides, about 50 polypeptides, about 100 polypeptides, about 500 polypeptides, about 1,000 polypeptides, about 2,000 polypeptides, about 5,000 polypeptides, about 10,000 polypeptides, about 20,000 polypeptides, or about 50,000 polypeptides. An in vitro transcription and translation reaction can generate at most about 10 polypeptides, about 50 polypeptides, about 100 polypeptides, about 500 polypeptides, about 1,000 polypeptides, about 2,000 polypeptides, about 5,000 polypeptides, about 10,000 polypeptides, about 20,000 polypeptides, about 50,000 polypeptides, or about 100,000 polypeptides.

An in vitro transcription and translation reaction can generate about 1 amino acid to about 10,000 amino acids in length. An in vitro transcription and translation reaction can generate about 1 amino acid to about 2 amino acids in length, about 1 amino acid to about 5 amino acids in length, about 1 amino acid to about 10 amino acids in length, about 1 amino acid to about 30 amino acids in length, about 1 amino acid to about 100 amino acids in length, about 1 amino acid to about 200 amino acids in length, about 1 amino acid to about 500 amino acids in length, about 1 amino acid to about 1,000 amino acids in length, about 1 amino acid to about 2,000 amino acids in length, about 1 amino acid to about 5,000 amino acids in length, about 1 amino acid to about 10,000 amino acids in length, about 2 amino acids in length to about 5 amino acids in length, about 2 amino acids in length to about 10 amino acids in length, about 2 amino acids in length to about 30 amino acids in length, about 2 amino acids in length to about 100 amino acids in length, about 2 amino acids in length to about 200 amino acids in length, about 2 amino acids in length to about 500 amino acids in length, about 2 amino acids in length to about 1,000 amino acids in length, about 2 amino acids in length to about 2,000 amino acids in length, about 2 amino acids in length to about 5,000 amino acids in length, about 2 amino acids in length to about 10,000 amino acids in length, about 5 amino acids in length to about 10 amino acids in length, about 5 amino acids in length to about 30 amino acids in length, about 5 amino acids in length to about 100 amino acids in length, about 5 amino acids in length to about 200 amino acids in length, about 5 amino acids in length to about 500 amino acids in length, about 5 amino acids in length to about 1,000 amino acids in length, about 5 amino acids in length to about 2,000 amino acids in length, about 5 amino acids in length to about 5,000 amino acids in length, about 5 amino acids in length to about 10,000 amino acids in length, about 10 amino acids in length to about 30 amino acids in length, about 10 amino acids in length to about 100 amino acids in length, about 10 amino acids in length to about 200 amino acids in length, about 10 amino acids in length to about 500 amino acids in length, about 10 amino acids in length to about 1,000 amino acids in length, about 10 amino acids in length to about 2,000 amino acids in length, about 10 amino acids in length to about 5,000 amino acids in length, about 10 amino acids in length to about 10,000 amino acids in length, about 30) amino acids in length to about 100 amino acids in length, about 30 amino acids in length to about 200 amino acids in length, about 30 amino acids in length to about 500 amino acids in length, about 30 amino acids in length to about 1,000 amino acids in length, about 30 amino acids in length to about 2,000 amino acids in length, about 30 amino acids in length to about 5,000 amino acids in length, about 30 amino acids in length to about 10,000 amino acids in length, about 100 amino acids in length to about 200 amino acids in length, about 100 amino acids in length to about 500 amino acids in length, about 100 amino acids in length to about 1,000 amino acids in length, about 100 amino acids in length to about 2,000 amino acids in length, about 100 amino acids in length to about 5,000 amino acids in length, about 100 amino acids in length to about 10,000 amino acids in length, about 200 amino acids in length to about 500 amino acids in length, about 200 amino acids in length to about 1,000 amino acids in length, about 200 amino acids in length to about 2.000 amino acids in length, about 200 amino acids in length to about 5,000 amino acids in length, about 200 amino acids in length to about 10,000 amino acids in length, about 500 amino acids in length to about 1,000 amino acids in length, about 500 amino acids in length to about 2,000 amino acids in length, about 500 amino acids in length to about 5,000 amino acids in length, about 500 amino acids in length to about 10,000 amino acids in length, about 1,000 amino acids in length to about 2,000 amino acids in length, about 1,000 amino acids in length to about 5,000 amino acids in length, about 1,000 amino acids in length to about 10,000 amino acids in length, about 2,000 amino acids in length to about 5,000 amino acids in length, about 2,000 amino acids in length to about 10,000 amino acids in length, or about 5,000 amino acids in length to about 10,000 amino acids in length. An in vitro transcription and translation reaction can generate about 1 amino acid, about 2 amino acids in length, about 5 amino acids in length, about 10 amino acids in length, about 30 amino acids in length, about 100 amino acids in length, about 200 amino acids in length, about 500 amino acids in length, about 1,000 amino acids in length, about 2,000 amino acids in length, about 5,000 amino acids in length, or about 10,000 amino acids in length. An in vitro transcription and translation reaction can generate at least about 1 amino acid, about 2 amino acids in length, about 5 amino acids in length, about 10 amino acids in length, about 30 amino acids in length, about 100 amino acids in length, about 200 amino acids in length, about 500 amino acids in length, about 1,000 amino acids in length, about 2,000 amino acids in length, or about 5,000 amino acids in length. An in vitro transcription and translation reaction can generate at most about 2 amino acids in length, about 5 amino acids in length, about 10 amino acids in length, about 30 amino acids in length, about 100 amino acids in length, about 200 amino acids in length, about 500 amino acids in length, about 1,000 amino acids in length, about 2,000 amino acids in length, about 5,000 amino acids in length, or about 10,000 amino acids in length.

The composition of the third emulsion is referred to in FIG. 4 and comprises one or more reagents including but not limited to said variant Cas polypeptide, a plasmid encoding a fluorescence reporter polypeptide, a FQ reporter molecule, cell-free transcription-translation system, and a double emulsion platform.

In one embodiment, reactions may be loaded into well plates and incubated in a microplate reader for iterative time measurements. Dual-channel imaging at regular timepoints can be used to reveal and decouple the cis and trans RNase activity of the Cas13/crRNA complex. The concentrations of all reaction components are titrated over an experimentally determined range. In one embodiment, off-target crRNAs and known inactive mutants of LwaCas13a can serve as negative activity controls when in complex with crRNAs.

For individual sequencing, droplets are sorted into different wells of a well plate to naturally lyse through contact with air, opening themselves to downstream analysis of the reaction contents. Borrowing from single-cell sequencing workflows, mRNA transcripts on beads are subject to reverse transcription (RT) via template switching. Resulting cDNA is amplified using the 15-bp handle as a universal primer binding site, then sequenced using conventional library preparation and next-generation sequencing (NGS) methods. Barcode identities will be traced back to their corresponding Cas13a variants through the previously established variant-barcode associations.

### Guide RNAs

The present disclosure also provides synthetic guide RNAs (sgRNAs). The guide RNAs hybridize to target nucleic acids such as target nucleic acids that are detectably labeled (or labeled such that a detectable label is released after nuclease cleavage). In certain embodiments, a library of guide RNAs is provided. Guide RNA generation for Cas12 and Cas13 is generally described by Arizti-Sanz, et al. in “Streamlined inactivation, amplification, and Cas13-based detection of SARS-COV-2.” Nat Commun 11, 5921 (2020). The guide RNAs may be screened against a wild-type or altered nuclease described herein to optimize one or more of the guide RNAs nuclease or target binding ability.

The library may comprise at least 10, 30, 50, 100, 500, 1000, 5,000, 10,000, 50,000, or 100,000 RNA molecules, each of which targets a different sequence in a target RNA or DNA. In this instance the target RNA or DNA can be the same gene targeted by multiple sgRNAs or multiple genes targeted by the same sgRNA. The library can also be in the form of a pool of at least two synthetic sgRNAs or an individual RNA in each well in a multi-well format. Variant RNAs can be guide RNAs or crRNAs.

The library may comprise of about 1 RNA molecule to about 100,000 RNA molecules, each of which targets a different sequence in a target RNA or DNA. The library may comprise about 1 RNA molecule to about 30 RNA molecules, about 1 RNA molecule to about 50) RNA molecules, about 1 RNA molecule to about 100 RNA molecules, about 1 RNA molecule to about 500 RNA molecules, about 1 RNA molecule to about 1,000 RNA molecules, about 1 RNA molecule to about 5,000 RNA molecules, about 1 RNA molecule to about 10,000 RNA molecules, about 1 RNA molecule to about 50,000 RNA molecules, about 1 RNA molecule to about 100,000 RNA molecules, about 30 RNA molecules to about 50 RNA molecules, about 30 RNA molecules to about 100 RNA molecules, about 30 RNA molecules to about 500) RNA molecules, about 30) RNA molecules to about 1,000 RNA molecules, about 30 RNA molecules to about 5,000 RNA molecules, about 30 RNA molecules to about 10,000 RNA molecules, about 30) RNA molecules to about 50,000 RNA molecules, about 30 RNA molecules to about 100,000 RNA molecules, about 50) RNA molecules to about 100 RNA molecules, about 50) RNA molecules to about 500) RNA molecules, about 50) RNA molecules to about 1,000 RNA molecules, about 50) RNA molecules to about 5,000 RNA molecules, about 50 RNA molecules to about 10,000 RNA molecules, about 50 RNA molecules to about 50,000 RNA molecules, about 50) RNA molecules to about 100,000 RNA molecules, about 100 RNA molecules to about 500) RNA molecules, about 100 RNA molecules to about 1,000 RNA molecules, about 100) RNA molecules to about 5,000 RNA molecules, about 100 RNA molecules to about 10,000 RNA molecules, about 100 RNA molecules to about 50,000 RNA molecules, about 100) RNA molecules to about 100,000 RNA molecules, about 500 RNA molecules to about 1,000 RNA molecules, about 500) RNA molecules to about 5,000 RNA molecules, about 500) RNA molecules to about 10,000 RNA molecules, about 500 RNA molecules to about 50,000 RNA molecules, about 500 RNA molecules to about 100,000 RNA molecules, about 1,000 RNA molecules to about 5,000 RNA molecules, about 1,000 RNA molecules to about 10,000 RNA molecules, about 1,000 RNA molecules to about 50,000 RNA molecules, about 1,000 RNA molecules to about 100,000 RNA molecules, about 5,000 RNA molecules to about 10,000 RNA molecules, about 5,000 RNA molecules to about 50,000 RNA molecules, about 5,000 RNA molecules to about 100,000 RNA molecules, about 10,000 RNA molecules to about 50,000 RNA molecules, about 10,000 RNA molecules to about 100,000 RNA molecules, or about 50,000 RNA molecules to about 100,000 RNA molecules, each of which targets a different sequence in a target RNA or DNA. The library may comprise about 1 RNA molecule, about 30) RNA molecules, about 50) RNA molecules, about 100 RNA molecules, about 500) RNA molecules, about 1,000 RNA molecules, about 5,000 RNA molecules, about 10,000 RNA molecules, about 50,000 RNA molecules, or about 100,000 RNA molecules. The library may comprise at least about 1 RNA molecule, about 30) RNA molecules, about 50) RNA molecules, about 100 RNA molecules, about 500) RNA molecules, about 1,000 RNA molecules, about 5,000 RNA molecules, about 10,000 RNA molecules, or about 50,000 RNA molecules, each of which targets a different sequence in a target RNA or DNA. The library may comprise at most about 30 RNA molecules, about 50 RNA molecules, about 100 RNA molecules, about 500 RNA molecules, about 1,000 RNA molecules, about 5,000 RNA molecules, about 10,000 RNA molecules, about 50,000 RNA molecules, or about 100,000 RNA molecules, each of which targets a different sequence in a target RNA or DNA.

For the method described herein any guide RNA may be used that effectively allows association of the Cas nuclease, and which may hybridize to a target molecule (e.g., target RNA).

Measuring the nuclease activity of said variant polypeptide is referred to in FIG. 5. Assay measurements may include but are not limited to cis nuclease activity, trans nuclease activity, or both. The cis and trans Cas nuclease activity is measured in a cell-free assay where changes in fluorescence molecule signals monitor the sequence-specific cis cleavage of a GFP transcript by the Cas/crRNA complex, whereas FQ reporter signal will monitor collateral trans cleavage upon Cas13 activation. crRNA spacers are designed to target RNA encoding green fluorescent protein (GFP) and knockdown efficiency is confirmed through SHERLOCK, a Cas13-based nucleic acid detection protocol. The overall cell-free reaction consists of three plasmids: (one expressing GFP, one expressing a GFP-targeting crRNA, and one expressing Cas13a), PURExpress solutions, and fluorophore-quencher (FQ) reporters.

Also contemplated are guide RNAs that comprise modifications from naturally occurring RNAs. In certain embodiments, the guide RNA comprises one or more of; a non-natural internucleoside linkage, a nucleic acid mimetic, a modified sugar moiety, and a modified nucleobase. In certain embodiments, the non-natural internucleoside linkage comprises one or more of; a phosphorothioate, a phosphoramidate, a non-phosphodiester, a heteroatom, a chiral phosphorothioate, a phosphorodithioate, a phosphotriester, an aminoalkylphosphotriester, a 3′-alkylene phosphonates, a 5′-alkylene phosphonate, a chiral phosphonate, a phosphinate, a 3′-amino phosphoramidate, an aminoalkylphosphoramidate, a phosphorodiamidate, a thionophosphoramidate, a thionoalkylphosphonate, a thionoalkylphosphotriester, a selenophosphate, and a boranophosphate. In certain embodiments, the nucleic acid mimetic comprises one or more of a peptide nucleic acid (PNA), morpholino nucleic acid, cyclohexenyl nucleic acid (CeNAs), or a locked nucleic acid (LNA). In certain embodiments, the modified sugar moiety comprises one or more of 2′-O-(2-methoxyethyl), 2′-dimethylaminooxyethoxy, 2′-dimethylaminoethoxyethoxy, 2′-O-methyl, and 2′-fluoro. In certain embodiments, the modified nucleobase comprises one or more of; a 5-methylcytosine; a 5-hydroxymethyl cytosine; a xanthine; a hypoxanthine; a 2-aminoadenine; a 6-methyl derivative of adenine; a 6-methyl derivative of guanine; a 2-propyl derivative of adenine; a 2-propyl derivative of guanine; a 2-thiouracil; a 2-thiothymine; a 2-thiocytosine; a 5-halouracil; a 5-halocytosine; a 5-propynyl uracil; a 5-propynyl cytosine; a 6-azo uracil; a 6-azo cytosine; a 6-azo thymine; a pseudouracil; a 4-thiouracil; an 8-halo; an 8-amino; an 8-thiol; an 8-thioalkyl; an 8-hydroxyl; a 5-halo; a 5-bromo; a 5-trifluoromethyl; a 5-substituted uracil; a 5-substituted cytosine; a 7-methylguanine; a 7-methyladenine; a 2-F-adenine; a 2-amino-adenine; an 8-azaguanine; an 8-azaadenine; a 7-deazaguanine; a 7-deazaadenine; a 3-deazaguanine; a 3-deazaadenine; a tricyclic pyrimidine; a phenoxazine cytidine; a phenothiazine cytidine; a substituted phenoxazine cytidine; a carbazole cytidine; a pyridoindole cytidine; a 7-deaza-adenine; a 7-deazaguanosine; a 2-aminopyridine; a 2-pyridone; a 5-substituted pyrimidine; a 6-azapyrimidine; an N-2, N-6 or O-6 substituted purine; a 2-aminopropyladenine; a 5-propynyluracil; and a 5-propynylcytosine.

### Screening Measurements Tools

Nuclease variants may be individually screened and isolated in each reaction compartments. The single variant screening setup comprises three syringe pumps, a microfluidic device, a camera, and a monitor for droplet visualization. Generated droplets can be imaged via fluorescence microscopy. The cis and trans Cas nuclease activity is measured in a cell-free assay where changes in fluorescence molecule signals monitor the sequence-specific cis cleavage of a GFP transcript by the Cas/crRNA complex, whereas FQ reporter signal will monitor collateral trans cleavage upon Cas13 activation. crRNA spacers are designed to target RNA encoding green fluorescent protein (GFP) and knockdown efficiency is confirmed through SHINE or SHERLOCK, both Cas13-based nucleic acid detection protocols.

Nuclease variants can be individually screened and isolated in each reaction compartments using a single variant approach. The single variant screening setup comprises three syringe pumps, a microfluidic device, a camera, and a monitor for droplet visualization. Generated droplets can be imaged via fluorescence microscopy. The cis and trans Cas nuclease activity is measured in a cell-free assay where changes in fluorescence molecule signals monitor the sequence-specific cis cleavage of a GFP transcript by the Cas/crRNA complex, whereas FQ reporter signal will monitor collateral trans cleavage upon Cas13 activation. crRNA spacers are designed to target RNA encoding green fluorescent protein (GFP) and knockdown efficiency is confirmed through SHINE or SHERLOCK, both Cas13-based nucleic acid detection protocols.

Double-emulsion droplet fluorescence may also be directly quantified through FACS. Droplets may be sorted into two replicates of a Cas droplet library: one with a GFP FITC-A vs. PE-A gate to isolate cis active variants, and once with an APC-A vs. PE-A gate to isolate trans active variants.

FACS screening of DE droplet fluorescence may indicate an increase in a variant's cis nuclease activity. FACS screening of DE droplet fluorescence may indicate a decrease in a variant's cis nuclease activity. FACS screening of DE droplet fluorescence may indicate an increase in a variant's trans nuclease activity. FACS screening of DE droplet fluorescence may indicate a decrease in a variant's trans nuclease activity. FACS screening of DE droplet fluorescence may indicate both an increase and later decrease in a variant's cis nuclease activity. FACS screening of DE droplet fluorescence may indicate both a decrease and later increase in a variant's cis nuclease activity. FACS screening of DE droplet fluorescence may indicate both an increase and later decrease in a variant's trans nuclease activity. FACS screening of DE droplet fluorescence may indicate both a decrease and later increase in a variant's trans nuclease activity. FACS screening of DE droplet fluorescence may indicate an increase in both a variant's cis and trans nuclease activity. FACS screening of DE droplet fluorescence may indicate a decrease in both a variant's cis and trans nuclease activity. FACS screening of DE droplet fluorescence may indicate an increase in a variant's cis and decrease in trans nuclease activity. FACS screening of DE droplet fluorescence may indicate an increase in a variant's trans and decrease in cis nuclease activity.

Cas Variants with Altered Nuclease Activity

Described herein is a variant Leptotrichia wadei Cas13 (LwaCas13) nuclease, wherein said variant LwaCas13 nuclease comprises an altered nuclease activity compared to an unaltered LwaCas13 in SEQ ID NO: 1. A variant Leptotrichia wadei Cas13 (LwaCas13) nuclease, wherein said variant LwaCas13 nuclease may comprise one or more of the following: an increase in said variant's cis nuclease activity, a decrease in said variant's cis nuclease activity, an increase in said variant's trans nuclease activity, and a decrease in said variant's trans nuclease activity.

The variant Leptotrichia wadei Cas13 (LwaCas13) nuclease may comprise one or more amino acid sequence modifications relative to SEQ ID NO: 1. The variant Leptotrichia wadei Cas13 (LwaCas13) nuclease may comprise one, two, three, four, five, six, seven, eight, nine, ten or more one or more amino acid sequence modifications relative to SEQ ID NO: 1.

The one or more amino acid sequence modifications may be in any one or more LwaCas13 domains selected from the list consisting of a HEPN-1 domain, a HEPN-2 domain, a Helical-1 domain, a Helical-2 domain, a Cas13a switch region, a Cas13 nuclease lobe, a Cas13 recognition lobe, a Cas13 linker region, a NTD, a Monolith region, and a Cas13 catalytic site. In certain embodiments, one or more amino acid sequence modifications relative to SEQ ID NO: 1 are in a HEPN nuclease activation domain. In certain embodiments, one or more amino acid sequence modifications relative to SEQ ID NO: 1 are in a RNA binding domain. In certain embodiments, two or more amino acid sequence modifications relative to SEQ ID NO: 1 are in a RNA binding domain and a HEPN nuclease activation domain

A variant LwaCas13 may possess about a 10% increase in cis nuclease activity to about a 1000% increase in cis nuclease activity. A variant LwaCas13 may possess about a 10% increase in cis nuclease activity to about a 50% increase in cis nuclease activity, about a 10% increase in cis nuclease activity to about a 100% increase in cis nuclease activity, about a 10% increase in cis nuclease activity to about a 200% increase in cis nuclease activity, about a 10% increase in cis nuclease activity to about a 500% increase in cis nuclease activity, about a 10% increase in cis nuclease activity to about a 1000% increase in cis nuclease activity, about a 50% increase in cis nuclease activity to about a 100% increase in cis nuclease activity, about a 50% increase in cis nuclease activity to about a 200% increase in cis nuclease activity, about a 50% increase in cis nuclease activity to about a 500% increase in cis nuclease activity, about a 50% increase in cis nuclease activity to about a 1000% increase in cis nuclease activity, about a 100% increase in cis nuclease activity to about a 200% increase in cis nuclease activity, about a 100% increase in cis nuclease activity to about a 500% increase in cis nuclease activity, about a 100% increase in cis nuclease activity to about a 1000% increase in cis nuclease activity, about a 200% increase in cis nuclease activity to about a 500% increase in cis nuclease activity, about a 200% increase in cis nuclease activity to about a 1000% increase in cis nuclease activity, or about a 500% increase in cis nuclease activity to about a 1000% increase in cis nuclease activity. A variant LwaCas13 may possess about a 10% increase in cis nuclease activity, about a 50% increase in cis nuclease activity, about a 100% increase in cis nuclease activity, about a 200% increase in cis nuclease activity, about a 500% increase in cis nuclease activity, or about a 1000% increase in cis nuclease activity. A variant LwaCas13 may possess at least about a 10% increase in cis nuclease activity, about a 50% increase in cis nuclease activity, about a 100% increase in cis nuclease activity, about a 200% increase in cis nuclease activity, or about a 500% increase in cis nuclease activity. A variant LwaCas13 may possess at most about a 50% increase in cis nuclease activity, about a 100% increase in cis nuclease activity, about a 200% increase in cis nuclease activity, about a 500% increase in cis nuclease activity, or about a 1000% increase in cis nuclease activity.

A variant LwaCas13 may possess about 1% decrease in cis nuclease activity to about 100% decrease in cis nuclease activity. A variant LwaCas13 may possess about 1% decrease in cis nuclease activity to about 2% decrease in cis nuclease activity, about 1% decrease in cis nuclease activity to about 5% decrease in cis nuclease activity, about 1% decrease in cis nuclease activity to about 10% decrease in cis nuclease activity, about 1% decrease in cis nuclease activity to about 25% decrease in cis nuclease activity, about 1% decrease in cis nuclease activity to about 50% decrease in cis nuclease activity, about 1% decrease in cis nuclease activity to about 75% decrease in cis nuclease activity, about 1% decrease in cis nuclease activity to about 99% decrease in cis nuclease activity, about 1% decrease in cis nuclease activity to about 100% decrease in cis nuclease activity, about 2% decrease in cis nuclease activity to about 5% decrease in cis nuclease activity, about 2% decrease in cis nuclease activity to about 10% decrease in cis nuclease activity, about 2% decrease in cis nuclease activity to about 25% decrease in cis nuclease activity, about 2% decrease in cis nuclease activity to about 50% decrease in cis nuclease activity, about 2% decrease in cis nuclease activity to about 75% decrease in cis nuclease activity, about 2% decrease in cis nuclease activity to about 99% decrease in cis nuclease activity, about 2% decrease in cis nuclease activity to about 100% decrease in cis nuclease activity, about 5% decrease in cis nuclease activity to about 10% decrease in cis nuclease activity, about 5% decrease in cis nuclease activity to about 25% decrease in cis nuclease activity, about 5% decrease in cis nuclease activity to about 50% decrease in cis nuclease activity, about 5% decrease in cis nuclease activity to about 75% decrease in cis nuclease activity, about 5% decrease in cis nuclease activity to about 99% decrease in cis nuclease activity, about 5% decrease in cis nuclease activity to about 100% decrease in cis nuclease activity, about 10% decrease in cis nuclease activity to about 25% decrease in cis nuclease activity, about 10% decrease in cis nuclease activity to about 50% decrease in cis nuclease activity, about 10% decrease in cis nuclease activity to about 75% decrease in cis nuclease activity, about 10% decrease in cis nuclease activity to about 99% decrease in cis nuclease activity, about 10% decrease in cis nuclease activity to about 100% decrease in cis nuclease activity, about 25% decrease in cis nuclease activity to about 50% decrease in cis nuclease activity, about 25% decrease in cis nuclease activity to about 75% decrease in cis nuclease activity, about 25% decrease in cis nuclease activity to about 99% decrease in cis nuclease activity, about 25% decrease in cis nuclease activity to about 100% decrease in cis nuclease activity, about 50% decrease in cis nuclease activity to about 75% decrease in cis nuclease activity, about 50% decrease in cis nuclease activity to about 99% decrease in cis nuclease activity, about 50% decrease in cis nuclease activity to about 100% decrease in cis nuclease activity, about 75% decrease in cis nuclease activity to about 99% decrease in cis nuclease activity, about 75% decrease in cis nuclease activity to about 100% decrease in cis nuclease activity, or about 99% decrease in cis nuclease activity to about 100% decrease in cis nuclease activity. A variant LwaCas13 may possess about 1% decrease in cis nuclease activity, about 2% decrease in cis nuclease activity, about 5% decrease in cis nuclease activity, about 10% decrease in cis nuclease activity, about 25% decrease in cis nuclease activity, about 50% decrease in cis nuclease activity, about 75% decrease in cis nuclease activity, about 99% decrease in cis nuclease activity, or about 100% decrease in cis nuclease activity. A variant LwaCas13 may possess at least about 1% decrease in cis nuclease activity, about 2% decrease in cis nuclease activity, about 5% decrease in cis nuclease activity, about 10% decrease in cis nuclease activity, about 25% decrease in cis nuclease activity, about 50% decrease in cis nuclease activity, about 75% decrease in cis nuclease activity, or about 99% decrease in cis nuclease activity. A variant LwaCas13 may possess at most about 2% decrease in cis nuclease activity, about 5% decrease in cis nuclease activity, about 10% decrease in cis nuclease activity, about 25% decrease in cis nuclease activity, about 50% decrease in cis nuclease activity, about 75% decrease in cis nuclease activity, about 99% decrease in cis nuclease activity, or about 100% decrease in cis nuclease activity

A variant LwaCas13 may possess about a 10% increase in trans nuclease activity to about a 1000% increase in trans nuclease activity. A variant LwaCas13 may possess about a 10% increase in trans nuclease activity to about a 50% increase in trans nuclease activity, about a 10% increase in trans nuclease activity to about a 100% increase in trans nuclease activity, about a 10% increase in trans nuclease activity to about a 200% increase in trans nuclease activity, about a 10% increase in trans nuclease activity to about a 500% increase in trans nuclease activity, about a 10% increase in trans nuclease activity to about a 1000% increase in trans nuclease activity, about a 50% increase in trans nuclease activity to about a 100% increase in trans nuclease activity, about a 50% increase in trans nuclease activity to about a 200% increase in trans nuclease activity, about a 50% increase in trans nuclease activity to about a 500% increase in trans nuclease activity, about a 50% increase in trans nuclease activity to about a 1000% increase in trans nuclease activity, about a 100% increase in trans nuclease activity to about a 200% increase in trans nuclease activity, about a 100% increase in trans nuclease activity to about a 500% increase in trans nuclease activity, about a 100% increase in trans nuclease activity to about a 1000% increase in trans nuclease activity, about a 200% increase in trans nuclease activity to about a 500% increase in trans nuclease activity, about a 200% increase in trans nuclease activity to about a 1000% increase in trans nuclease activity, or about a 500% increase in trans nuclease activity to about a 1000% increase in trans nuclease activity. A variant LwaCas13 may possess about a 10% increase in trans nuclease activity, about a 50% increase in trans nuclease activity, about a 100% increase in trans nuclease activity, about a 200% increase in trans nuclease activity, about a 500% increase in trans nuclease activity, or about a 1000% increase in trans nuclease activity. A variant LwaCas13 may possess at least about a 10% increase in trans nuclease activity, about a 50% increase in trans nuclease activity, about a 100% increase in trans nuclease activity, about a 200% increase in trans nuclease activity, or about a 500% increase in trans nuclease activity. A variant LwaCas13 may possess at most about a 50% increase in trans nuclease activity, about a 100% increase in trans nuclease activity, about a 200% increase in trans nuclease activity, about a 500% increase in trans nuclease activity, or about a 1000% increase in trans nuclease activity.

A variant LwaCas13 may possess about 1% decrease in trans nuclease activity to about 100% decrease in trans nuclease activity. A variant LwaCas13 may possess about 1% decrease in trans nuclease activity to about 2% decrease in trans nuclease activity, about 1% decrease in trans nuclease activity to about 5% decrease in trans nuclease activity, about 1% decrease in trans nuclease activity to about 10% decrease in trans nuclease activity, about 1% decrease in trans nuclease activity to about 25% decrease in trans nuclease activity, about 1% decrease in trans nuclease activity to about 50% decrease in trans nuclease activity, about 1% decrease in trans nuclease activity to about 75% decrease in trans nuclease activity, about 1% decrease in trans nuclease activity to about 99% decrease in trans nuclease activity, about 1% decrease in trans nuclease activity to about 100% decrease in trans nuclease activity, about 2% decrease in trans nuclease activity to about 5% decrease in trans nuclease activity, about 2% decrease in trans nuclease activity to about 10% decrease in trans nuclease activity, about 2% decrease in trans nuclease activity to about 25% decrease in trans nuclease activity, about 2% decrease in trans nuclease activity to about 50% decrease in trans nuclease activity, about 2% decrease in trans nuclease activity to about 75% decrease in trans nuclease activity, about 2% decrease in trans nuclease activity to about 99% decrease in trans nuclease activity, about 2% decrease in trans nuclease activity to about 100% decrease in trans nuclease activity, about 5% decrease in trans nuclease activity to about 10% decrease in trans nuclease activity, about 5% decrease in trans nuclease activity to about 25% decrease in trans nuclease activity, about 5% decrease in trans nuclease activity to about 50% decrease in trans nuclease activity, about 5% decrease in trans nuclease activity to about 75% decrease in trans nuclease activity, about 5% decrease in trans nuclease activity to about 99% decrease in trans nuclease activity, about 5% decrease in trans nuclease activity to about 100% decrease in trans nuclease activity, about 10% decrease in trans nuclease activity to about 25% decrease in trans nuclease activity, about 10% decrease in trans nuclease activity to about 50% decrease in trans nuclease activity, about 10% decrease in trans nuclease activity to about 75% decrease in trans nuclease activity, about 10% decrease in trans nuclease activity to about 99% decrease in trans nuclease activity, about 10% decrease in trans nuclease activity to about 100% decrease in trans nuclease activity, about 25% decrease in trans nuclease activity to about 50% decrease in trans nuclease activity, about 25% decrease in trans nuclease activity to about 75% decrease in trans nuclease activity, about 25% decrease in trans nuclease activity to about 99% decrease in trans nuclease activity, about 25% decrease in trans nuclease activity to about 100% decrease in trans nuclease activity, about 50% decrease in trans nuclease activity to about 75% decrease in trans nuclease activity, about 50% decrease in trans nuclease activity to about 99% decrease in trans nuclease activity, about 50% decrease in trans nuclease activity to about 100% decrease in trans nuclease activity, about 75% decrease in trans nuclease activity to about 99% decrease in trans nuclease activity, about 75% decrease in trans nuclease activity to about 100% decrease in trans nuclease activity, or about 99% decrease in trans nuclease activity to about 100% decrease in trans nuclease activity. A variant LwaCas13 may possess about 1% decrease in trans nuclease activity, about 2% decrease in trans nuclease activity, about 5% decrease in trans nuclease activity, about 10% decrease in trans nuclease activity, about 25% decrease in trans nuclease activity, about 50% decrease in trans nuclease activity, about 75% decrease in trans nuclease activity, about 99% decrease in trans nuclease activity, or about 100% decrease in trans nuclease activity. A variant LwaCas13 may possess at least about 1% decrease in trans nuclease activity, about 2% decrease in trans nuclease activity, about 5% decrease in trans nuclease activity, about 10% decrease in trans nuclease activity, about 25% decrease in trans nuclease activity, about 50% decrease in trans nuclease activity, about 75% decrease in trans nuclease activity, or about 99% decrease in trans nuclease activity. A variant LwaCas13 may possess at most about 2% decrease in trans nuclease activity, about 5% decrease in trans nuclease activity, about 10% decrease in trans nuclease activity, about 25% decrease in trans nuclease activity, about 50% decrease in trans nuclease activity, about 75% decrease in trans nuclease activity, about 99% decrease in trans nuclease activity, or about 100% decrease in trans nuclease activity. A variant Leptotrichia buccalis Cas13 (LbuCas13) nuclease, wherein said variant LbuCas13 nuclease comprises an altered nuclease activity compared to an unaltered LbuCas13 in SEQ ID NO: 3. A variant Leptotrichia buccalis Cas13 (LbuCas13) nuclease, wherein said variant LbuCas13 nuclease may comprise one or more of the following; an increase in said variant's cis nuclease activity, a decrease in said variant's cis nuclease activity, an increase in said variant's trans nuclease activity, and a decrease in said variant's trans nuclease activity.

The variant Leptotrichia buccalis Cas13 (LbuCas13) nuclease may comprise one or more amino acid sequence modifications relative to SEQ ID NO: 3. The variant Leptotrichia buccalis Cas13 (LbuCas13) nuclease may comprise one, two, three, four, five, six, seven, eight, nine, ten or more one or more amino acid sequence modifications relative to SEQ ID NO: 3.

The one or more amino acid sequence modifications may be in any one or more LbuCas13 domains selected from the list consisting of a HEPN-1 domain, a HEPN-2 domain, a Helical-1 domain, a Helical-2 domain, a Cas13a switch region, a Cas13 nuclease lobe, a Cas13 recognition lobe, a Cas13 linker region, a NTD, a Monolith region, and a Cas13 catalytic site. In certain embodiments, one or more amino acid sequence modifications relative to SEQ ID NO: 3 are in a HEPN nuclease activation domain. In certain embodiments, one or more amino acid sequence modifications relative to SEQ ID NO: 3 are in a RNA binding domain. In certain embodiments, two or more amino acid sequence modifications relative to SEQ ID NO: 3 are in a RNA binding domain and a HEPN nuclease activation domain.

A variant LbuCas13 may possess about a 10% increase in cis nuclease activity to about a 1000% increase in cis nuclease activity. A variant LbuCas13 may possess about a 10% increase in cis nuclease activity to about a 50% increase in cis nuclease activity, about a 10% increase in cis nuclease activity to about a 100% increase in cis nuclease activity, about a 10% increase in cis nuclease activity to about a 200% increase in cis nuclease activity, about a 10% increase in cis nuclease activity to about a 500% increase in cis nuclease activity, about a 10% increase in cis nuclease activity to about a 1000% increase in cis nuclease activity, about a 50% increase in cis nuclease activity to about a 100% increase in cis nuclease activity, about a 50% increase in cis nuclease activity to about a 200% increase in cis nuclease activity, about a 50% increase in cis nuclease activity to about a 500% increase in cis nuclease activity, about a 50% increase in cis nuclease activity to about a 1000% increase in cis nuclease activity, about a 100% increase in cis nuclease activity to about a 200% increase in cis nuclease activity, about a 100% increase in cis nuclease activity to about a 500% increase in cis nuclease activity, about a 100% increase in cis nuclease activity to about a 1000% increase in cis nuclease activity, about a 200% increase in cis nuclease activity to about a 500% increase in cis nuclease activity, about a 200% increase in cis nuclease activity to about a 1000% increase in cis nuclease activity, or about a 500% increase in cis nuclease activity to about a 1000% increase in cis nuclease activity. A variant LbuCas13 may possess about a 10% increase in cis nuclease activity, about a 50% increase in cis nuclease activity, about a 100% increase in cis nuclease activity, about a 200% increase in cis nuclease activity, about a 500% increase in cis nuclease activity, or about a 1000% increase in cis nuclease activity. A variant LbuCas13 may possess at least about a 10% increase in cis nuclease activity, about a 50% increase in cis nuclease activity, about a 100% increase in cis nuclease activity, about a 200% increase in cis nuclease activity, or about a 500% increase in cis nuclease activity. A variant LbuCas13 may possess at most about a 50% increase in cis nuclease activity, about a 100% increase in cis nuclease activity, about a 200% increase in cis nuclease activity, about a 500% increase in cis nuclease activity, or about a 1000% increase in cis nuclease activity.

A variant LbuCas13 may possess about 1% decrease in cis nuclease activity to about 100% decrease in cis nuclease activity. A variant LbuCas13 may possess about 1% decrease in cis nuclease activity to about 2% decrease in cis nuclease activity, about 1% decrease in cis nuclease activity to about 5% decrease in cis nuclease activity, about 1% decrease in cis nuclease activity to about 10% decrease in cis nuclease activity, about 1% decrease in cis nuclease activity to about 25% decrease in cis nuclease activity, about 1% decrease in cis nuclease activity to about 50% decrease in cis nuclease activity, about 1% decrease in cis nuclease activity to about 75% decrease in cis nuclease activity, about 1% decrease in cis nuclease activity to about 99% decrease in cis nuclease activity, about 1% decrease in cis nuclease activity to about 100% decrease in cis nuclease activity, about 2% decrease in cis nuclease activity to about 5% decrease in cis nuclease activity, about 2% decrease in cis nuclease activity to about 10% decrease in cis nuclease activity, about 2% decrease in cis nuclease activity to about 25% decrease in cis nuclease activity, about 2% decrease in cis nuclease activity to about 50% decrease in cis nuclease activity, about 2% decrease in cis nuclease activity to about 75% decrease in cis nuclease activity, about 2% decrease in cis nuclease activity to about 99% decrease in cis nuclease activity, about 2% decrease in cis nuclease activity to about 100% decrease in cis nuclease activity, about 5% decrease in cis nuclease activity to about 10% decrease in cis nuclease activity, about 5% decrease in cis nuclease activity to about 25% decrease in cis nuclease activity, about 5% decrease in cis nuclease activity to about 50% decrease in cis nuclease activity, about 5% decrease in cis nuclease activity to about 75% decrease in cis nuclease activity, about 5% decrease in cis nuclease activity to about 99% decrease in cis nuclease activity, about 5% decrease in cis nuclease activity to about 100% decrease in cis nuclease activity, about 10% decrease in cis nuclease activity to about 25% decrease in cis nuclease activity, about 10% decrease in cis nuclease activity to about 50% decrease in cis nuclease activity, about 10% decrease in cis nuclease activity to about 75% decrease in cis nuclease activity, about 10% decrease in cis nuclease activity to about 99% decrease in cis nuclease activity, about 10% decrease in cis nuclease activity to about 100% decrease in cis nuclease activity, about 25% decrease in cis nuclease activity to about 50% decrease in cis nuclease activity, about 25% decrease in cis nuclease activity to about 75% decrease in cis nuclease activity, about 25% decrease in cis nuclease activity to about 99% decrease in cis nuclease activity, about 25% decrease in cis nuclease activity to about 100% decrease in cis nuclease activity, about 50% decrease in cis nuclease activity to about 75% decrease in cis nuclease activity, about 50% decrease in cis nuclease activity to about 99% decrease in cis nuclease activity, about 50% decrease in cis nuclease activity to about 100% decrease in cis nuclease activity, about 75% decrease in cis nuclease activity to about 99% decrease in cis nuclease activity, about 75% decrease in cis nuclease activity to about 100% decrease in cis nuclease activity, or about 99% decrease in cis nuclease activity to about 100% decrease in cis nuclease activity. A variant LbuCas13 may possess about 1% decrease in cis nuclease activity, about 2% decrease in cis nuclease activity, about 5% decrease in cis nuclease activity, about 10% decrease in cis nuclease activity, about 25% decrease in cis nuclease activity, about 50% decrease in cis nuclease activity, about 75% decrease in cis nuclease activity, about 99% decrease in cis nuclease activity, or about 100% decrease in cis nuclease activity. A variant LbuCas13 may possess at least about 1% decrease in cis nuclease activity, about 2% decrease in cis nuclease activity, about 5% decrease in cis nuclease activity, about 10% decrease in cis nuclease activity, about 25% decrease in cis nuclease activity, about 50% decrease in cis nuclease activity, about 75% decrease in cis nuclease activity, or about 99% decrease in cis nuclease activity. A variant LbuCas13 may possess at most about 2% decrease in cis nuclease activity, about 5% decrease in cis nuclease activity, about 10% decrease in cis nuclease activity, about 25% decrease in cis nuclease activity, about 50% decrease in cis nuclease activity, about 75% decrease in cis nuclease activity, about 99% decrease in cis nuclease activity, or about 100% decrease in cis nuclease activity

A variant LbuCas13 may possess about a 10% increase in trans nuclease activity to about a 1000% increase in trans nuclease activity. A variant LbuCas13 may possess about a 10% increase in trans nuclease activity to about a 50% increase in trans nuclease activity, about a 10% increase in trans nuclease activity to about a 100% increase in trans nuclease activity, about a 10% increase in trans nuclease activity to about a 200% increase in trans nuclease activity, about a 10% increase in trans nuclease activity to about a 500% increase in trans nuclease activity, about a 10% increase in trans nuclease activity to about a 1000% increase in trans nuclease activity, about a 50% increase in trans nuclease activity to about a 100% increase in trans nuclease activity, about a 50% increase in trans nuclease activity to about a 200% increase in trans nuclease activity, about a 50% increase in trans nuclease activity to about a 500% increase in trans nuclease activity, about a 50% increase in trans nuclease activity to about a 1000% increase in trans nuclease activity, about a 100% increase in trans nuclease activity to about a 200% increase in trans nuclease activity, about a 100% increase in trans nuclease activity to about a 500% increase in trans nuclease activity, about a 100% increase in trans nuclease activity to about a 1000% increase in trans nuclease activity, about a 200% increase in trans nuclease activity to about a 500% increase in trans nuclease activity, about a 200% increase in trans nuclease activity to about a 1000% increase in trans nuclease activity, or about a 500% increase in trans nuclease activity to about a 1000% increase in trans nuclease activity. A variant LbuCas13 may possess about a 10% increase in trans nuclease activity, about a 50% increase in trans nuclease activity, about a 100% increase in trans nuclease activity, about a 200% increase in trans nuclease activity, about a 500% increase in trans nuclease activity, or about a 1000% increase in trans nuclease activity. A variant LbuCas13 may possess at least about a 10% increase in trans nuclease activity, about a 50% increase in trans nuclease activity, about a 100% increase in trans nuclease activity, about a 200% increase in trans nuclease activity, or about a 500% increase in trans nuclease activity. A variant LbuCas13 may possess at most about a 50% increase in trans nuclease activity, about a 100% increase in trans nuclease activity, about a 200% increase in trans nuclease activity, about a 500% increase in trans nuclease activity, or about a 1000% increase in trans nuclease activity.

A variant LbuCas13 may possess about 1% decrease in trans nuclease activity to about 100% decrease in trans nuclease activity. A variant LbuCas13 may possess about 1% decrease in trans nuclease activity to about 2% decrease in trans nuclease activity, about 1% decrease in trans nuclease activity to about 5% decrease in trans nuclease activity, about 1% decrease in trans nuclease activity to about 10% decrease in trans nuclease activity, about 1% decrease in trans nuclease activity to about 25% decrease in trans nuclease activity, about 1% decrease in trans nuclease activity to about 50% decrease in trans nuclease activity, about 1% decrease in trans nuclease activity to about 75% decrease in trans nuclease activity, about 1% decrease in trans nuclease activity to about 99% decrease in trans nuclease activity, about 1% decrease in trans nuclease activity to about 100% decrease in trans nuclease activity, about 2% decrease in trans nuclease activity to about 5% decrease in trans nuclease activity, about 2% decrease in trans nuclease activity to about 10% decrease in trans nuclease activity, about 2% decrease in trans nuclease activity to about 25% decrease in trans nuclease activity, about 2% decrease in trans nuclease activity to about 50% decrease in trans nuclease activity, about 2% decrease in trans nuclease activity to about 75% decrease in trans nuclease activity, about 2% decrease in trans nuclease activity to about 99% decrease in trans nuclease activity, about 2% decrease in trans nuclease activity to about 100% decrease in trans nuclease activity, about 5% decrease in trans nuclease activity to about 10% decrease in trans nuclease activity, about 5% decrease in trans nuclease activity to about 25% decrease in trans nuclease activity, about 5% decrease in trans nuclease activity to about 50% decrease in trans nuclease activity, about 5% decrease in trans nuclease activity to about 75% decrease in trans nuclease activity, about 5% decrease in trans nuclease activity to about 99% decrease in trans nuclease activity, about 5% decrease in trans nuclease activity to about 100% decrease in trans nuclease activity, about 10% decrease in trans nuclease activity to about 25% decrease in trans nuclease activity, about 10% decrease in trans nuclease activity to about 50% decrease in trans nuclease activity, about 10% decrease in trans nuclease activity to about 75% decrease in trans nuclease activity, about 10% decrease in trans nuclease activity to about 99% decrease in trans nuclease activity, about 10% decrease in trans nuclease activity to about 100% decrease in trans nuclease activity, about 25% decrease in trans nuclease activity to about 50% decrease in trans nuclease activity, about 25% decrease in trans nuclease activity to about 75% decrease in trans nuclease activity, about 25% decrease in trans nuclease activity to about 99% decrease in trans nuclease activity, about 25% decrease in trans nuclease activity to about 100% decrease in trans nuclease activity, about 50% decrease in trans nuclease activity to about 75% decrease in trans nuclease activity, about 50% decrease in trans nuclease activity to about 99% decrease in trans nuclease activity, about 50% decrease in trans nuclease activity to about 100% decrease in trans nuclease activity, about 75% decrease in trans nuclease activity to about 99% decrease in trans nuclease activity, about 75% decrease in trans nuclease activity to about 100% decrease in trans nuclease activity, or about 99% decrease in trans nuclease activity to about 100% decrease in trans nuclease activity. A variant LbuCas13 may possess about 1% decrease in trans nuclease activity, about 2% decrease in trans nuclease activity, about 5% decrease in trans nuclease activity, about 10% decrease in trans nuclease activity, about 25% decrease in trans nuclease activity, about 50% decrease in trans nuclease activity, about 75% decrease in trans nuclease activity, about 99% decrease in trans nuclease activity, or about 100% decrease in trans nuclease activity. A variant LbuCas13 may possess at least about 1% decrease in trans nuclease activity, about 2% decrease in trans nuclease activity, about 5% decrease in trans nuclease activity, about 10% decrease in trans nuclease activity, about 25% decrease in trans nuclease activity, about 50% decrease in trans nuclease activity, about 75% decrease in trans nuclease activity, or about 99% decrease in trans nuclease activity. A variant LbuCas13 may possess at most about 2% decrease in trans nuclease activity, about 5% decrease in trans nuclease activity, about 10% decrease in trans nuclease activity, about 25% decrease in trans nuclease activity, about 50% decrease in trans nuclease activity, about 75% decrease in trans nuclease activity, about 99% decrease in trans nuclease activity, or about 100% decrease in trans nuclease activity.

“CasRx”) nuclease, wherein said variant RfxCas13d nuclease comprises an altered nuclease activity compared to an unaltered RfxCas13d in SEQ ID NO: 5. A variant Ruminococcus flavefaciens XPD3002 (RfxCas13d) nuclease, wherein said variant RfxCas13d nuclease may comprise one or more of the following; an increase in said variant's cis nuclease activity, a decrease in said variant's cis nuclease activity, an increase in said variant's trans nuclease activity, and a decrease in said variant's trans nuclease activity.

The variant Ruminococcus flavefaciens XPD3002 (RfxCas13d) nuclease may comprise one or more amino acid sequence modifications relative to SEQ ID NO: 5. The variant Ruminococcus flavefaciens XPD3002 (RfxCas13d) nuclease may comprise one, two, three, four, five, six, seven, eight, nine, ten or more one or more amino acid sequence modifications relative to SEQ ID NO: 5.

The one or more amino acid sequence modifications may be in any one or more RfxCas13d domains selected from the list consisting of a HEPN-1 domain, a HEPN-2 domain, a Helical-1 domain, a Helical-2 domain, a Cas13 switch region, a Cas13 nuclease lobe, a Cas13 recognition lobe, a Cas13 linker region, a NTD, a Monolith region, and a Cas13 catalytic site. In certain embodiments, one or more amino acid sequence modifications relative to SEQ ID NO: 5 are in a HEPN nuclease activation domain. In certain embodiments, one or more amino acid sequence modifications relative to SEQ ID NO: 5 are in a RNA binding domain. In certain embodiments, two or more amino acid sequence modifications relative to SEQ ID NO: 5 are in a RNA binding domain and a HEPN nuclease activation domain

A variant RfxCas13d may possess about a 10% increase in cis nuclease activity to about a 1000% increase in cis nuclease activity. A variant RfxCas13d may possess about a 10% increase in cis nuclease activity to about a 50% increase in cis nuclease activity, about a 10% increase in cis nuclease activity to about a 100% increase in cis nuclease activity, about a 10% increase in cis nuclease activity to about a 200% increase in cis nuclease activity, about a 10% increase in cis nuclease activity to about a 500% increase in cis nuclease activity, about a 10% increase in cis nuclease activity to about a 1000% increase in cis nuclease activity, about a 50% increase in cis nuclease activity to about a 100% increase in cis nuclease activity, about a 50% increase in cis nuclease activity to about a 200% increase in cis nuclease activity, about a 50% increase in cis nuclease activity to about a 500% increase in cis nuclease activity, about a 50% increase in cis nuclease activity to about a 1000% increase in cis nuclease activity, about a 100% increase in cis nuclease activity to about a 200% increase in cis nuclease activity, about a 100% increase in cis nuclease activity to about a 500% increase in cis nuclease activity, about a 100% increase in cis nuclease activity to about a 1000% increase in cis nuclease activity, about a 200% increase in cis nuclease activity to about a 500% increase in cis nuclease activity, about a 200% increase in cis nuclease activity to about a 1000% increase in cis nuclease activity, or about a 500% increase in cis nuclease activity to about a 1000% increase in cis nuclease activity. A variant RfxCas13d may possess about a 10% increase in cis nuclease activity, about a 50% increase in cis nuclease activity, about a 100% increase in cis nuclease activity, about a 200% increase in cis nuclease activity, about a 500% increase in cis nuclease activity, or about a 1000% increase in cis nuclease activity. A variant RfxCas13d may possess at least about a 10% increase in cis nuclease activity, about a 50% increase in cis nuclease activity, about a 100% increase in cis nuclease activity, about a 200% increase in cis nuclease activity, or about a 500% increase in cis nuclease activity. A variant RfxCas13d may possess at most about a 50% increase in cis nuclease activity, about a 100% increase in cis nuclease activity, about a 200% increase in cis nuclease activity, about a 500% increase in cis nuclease activity, or about a 1000% increase in cis nuclease activity.

A variant RfxCas13d may possess about 1% decrease in cis nuclease activity to about 100% decrease in cis nuclease activity. A variant RfxCas13d may possess about 1% decrease in cis nuclease activity to about 2% decrease in cis nuclease activity, about 1% decrease in cis nuclease activity to about 5% decrease in cis nuclease activity, about 1% decrease in cis nuclease activity to about 10% decrease in cis nuclease activity, about 1% decrease in cis nuclease activity to about 25% decrease in cis nuclease activity, about 1% decrease in cis nuclease activity to about 50% decrease in cis nuclease activity, about 1% decrease in cis nuclease activity to about 75% decrease in cis nuclease activity, about 1% decrease in cis nuclease activity to about 99% decrease in cis nuclease activity, about 1% decrease in cis nuclease activity to about 100% decrease in cis nuclease activity, about 2% decrease in cis nuclease activity to about 5% decrease in cis nuclease activity, about 2% decrease in cis nuclease activity to about 10% decrease in cis nuclease activity, about 2% decrease in cis nuclease activity to about 25% decrease in cis nuclease activity, about 2% decrease in cis nuclease activity to about 50% decrease in cis nuclease activity, about 2% decrease in cis nuclease activity to about 75% decrease in cis nuclease activity, about 2% decrease in cis nuclease activity to about 99% decrease in cis nuclease activity, about 2% decrease in cis nuclease activity to about 100% decrease in cis nuclease activity, about 5% decrease in cis nuclease activity to about 10% decrease in cis nuclease activity, about 5% decrease in cis nuclease activity to about 25% decrease in cis nuclease activity, about 5% decrease in cis nuclease activity to about 50% decrease in cis nuclease activity, about 5% decrease in cis nuclease activity to about 75% decrease in cis nuclease activity, about 5% decrease in cis nuclease activity to about 99% decrease in cis nuclease activity, about 5% decrease in cis nuclease activity to about 100% decrease in cis nuclease activity, about 10% decrease in cis nuclease activity to about 25% decrease in cis nuclease activity, about 10% decrease in cis nuclease activity to about 50% decrease in cis nuclease activity, about 10% decrease in cis nuclease activity to about 75% decrease in cis nuclease activity, about 10% decrease in cis nuclease activity to about 99% decrease in cis nuclease activity, about 10% decrease in cis nuclease activity to about 100% decrease in cis nuclease activity, about 25% decrease in cis nuclease activity to about 50% decrease in cis nuclease activity, about 25% decrease in cis nuclease activity to about 75% decrease in cis nuclease activity, about 25% decrease in cis nuclease activity to about 99% decrease in cis nuclease activity, about 25% decrease in cis nuclease activity to about 100% decrease in cis nuclease activity, about 50% decrease in cis nuclease activity to about 75% decrease in cis nuclease activity, about 50% decrease in cis nuclease activity to about 99% decrease in cis nuclease activity, about 50% decrease in cis nuclease activity to about 100% decrease in cis nuclease activity, about 75% decrease in cis nuclease activity to about 99% decrease in cis nuclease activity, about 75% decrease in cis nuclease activity to about 100% decrease in cis nuclease activity, or about 99% decrease in cis nuclease activity to about 100% decrease in cis nuclease activity. A variant RfxCas13d may possess about 1% decrease in cis nuclease activity, about 2% decrease in cis nuclease activity, about 5% decrease in cis nuclease activity, about 10% decrease in cis nuclease activity, about 25% decrease in cis nuclease activity, about 50% decrease in cis nuclease activity, about 75% decrease in cis nuclease activity, about 99% decrease in cis nuclease activity, or about 100% decrease in cis nuclease activity. A variant RfxCas13d may possess at least about 1% decrease in cis nuclease activity, about 2% decrease in cis nuclease activity, about 5% decrease in cis nuclease activity, about 10% decrease in cis nuclease activity, about 25% decrease in cis nuclease activity, about 50% decrease in cis nuclease activity, about 75% decrease in cis nuclease activity, or about 99% decrease in cis nuclease activity. A variant RfxCas13d may possess at most about 2% decrease in cis nuclease activity, about 5% decrease in cis nuclease activity, about 10% decrease in cis nuclease activity, about 25% decrease in cis nuclease activity, about 50% decrease in cis nuclease activity, about 75% decrease in cis nuclease activity, about 99% decrease in cis nuclease activity, or about 100% decrease in cis nuclease activity

A variant RfxCas13d may possess about a 10% increase in trans nuclease activity to about a 1000% increase in trans nuclease activity. A variant RfxCas13d may possess about a 10% increase in trans nuclease activity to about a 50% increase in trans nuclease activity, about a 10% increase in trans nuclease activity to about a 100% increase in trans nuclease activity, about a 10% increase in trans nuclease activity to about a 200% increase in trans nuclease activity, about a 10% increase in trans nuclease activity to about a 500% increase in trans nuclease activity, about a 10% increase in trans nuclease activity to about a 1000% increase in trans nuclease activity, about a 50% increase in trans nuclease activity to about a 100% increase in trans nuclease activity, about a 50% increase in trans nuclease activity to about a 200% increase in trans nuclease activity, about a 50% increase in trans nuclease activity to about a 500% increase in trans nuclease activity, about a 50% increase in trans nuclease activity to about a 1000% increase in trans nuclease activity, about a 100% increase in trans nuclease activity to about a 200% increase in trans nuclease activity, about a 100% increase in trans nuclease activity to about a 500% increase in trans nuclease activity, about a 100% increase in trans nuclease activity to about a 1000% increase in trans nuclease activity, about a 200% increase in trans nuclease activity to about a 500% increase in trans nuclease activity, about a 200% increase in trans nuclease activity to about a 1000% increase in trans nuclease activity, or about a 500% increase in trans nuclease activity to about a 1000% increase in trans nuclease activity. A variant RfxCas13d may possess about a 10% increase in trans nuclease activity, about a 50% increase in trans nuclease activity, about a 100% increase in trans nuclease activity, about a 200% increase in trans nuclease activity, about a 500% increase in trans nuclease activity, or about a 1000% increase in trans nuclease activity. A variant RfxCas13d may possess at least about a 10% increase in trans nuclease activity, about a 50% increase in trans nuclease activity, about a 100% increase in trans nuclease activity, about a 200% increase in trans nuclease activity, or about a 500% increase in trans nuclease activity. A variant RfxCas13d may possess at most about a 50% increase in trans nuclease activity, about a 100% increase in trans nuclease activity, about a 200% increase in trans nuclease activity, about a 500% increase in trans nuclease activity, or about a 1000% increase in trans nuclease activity.

A variant RfxCas13d may possess about 1% decrease in trans nuclease activity to about 100% decrease in trans nuclease activity. A variant RfxCas13d may possess about 1% decrease in trans nuclease activity to about 2% decrease in trans nuclease activity, about 1% decrease in trans nuclease activity to about 5% decrease in trans nuclease activity, about 1% decrease in trans nuclease activity to about 10% decrease in trans nuclease activity, about 1% decrease in trans nuclease activity to about 25% decrease in trans nuclease activity, about 1% decrease in trans nuclease activity to about 50% decrease in trans nuclease activity, about 1% decrease in trans nuclease activity to about 75% decrease in trans nuclease activity, about 1% decrease in trans nuclease activity to about 99% decrease in trans nuclease activity, about 1% decrease in trans nuclease activity to about 100% decrease in trans nuclease activity, about 2% decrease in trans nuclease activity to about 5% decrease in trans nuclease activity, about 2% decrease in trans nuclease activity to about 10% decrease in trans nuclease activity, about 2% decrease in trans nuclease activity to about 25% decrease in trans nuclease activity, about 2% decrease in trans nuclease activity to about 50% decrease in trans nuclease activity, about 2% decrease in trans nuclease activity to about 75% decrease in trans nuclease activity, about 2% decrease in trans nuclease activity to about 99% decrease in trans nuclease activity, about 2% decrease in trans nuclease activity to about 100% decrease in trans nuclease activity, about 5% decrease in trans nuclease activity to about 10% decrease in trans nuclease activity, about 5% decrease in trans nuclease activity to about 25% decrease in trans nuclease activity, about 5% decrease in trans nuclease activity to about 50% decrease in trans nuclease activity, about 5% decrease in trans nuclease activity to about 75% decrease in trans nuclease activity, about 5% decrease in trans nuclease activity to about 99% decrease in trans nuclease activity, about 5% decrease in trans nuclease activity to about 100% decrease in trans nuclease activity, about 10% decrease in trans nuclease activity to about 25% decrease in trans nuclease activity, about 10% decrease in trans nuclease activity to about 50% decrease in trans nuclease activity, about 10% decrease in trans nuclease activity to about 75% decrease in trans nuclease activity, about 10% decrease in trans nuclease activity to about 99% decrease in trans nuclease activity, about 10% decrease in trans nuclease activity to about 100% decrease in trans nuclease activity, about 25% decrease in trans nuclease activity to about 50% decrease in trans nuclease activity, about 25% decrease in trans nuclease activity to about 75% decrease in trans nuclease activity, about 25% decrease in trans nuclease activity to about 99% decrease in trans nuclease activity, about 25% decrease in trans nuclease activity to about 100% decrease in trans nuclease activity, about 50% decrease in trans nuclease activity to about 75% decrease in trans nuclease activity, about 50% decrease in trans nuclease activity to about 99% decrease in trans nuclease activity, about 50% decrease in trans nuclease activity to about 100% decrease in trans nuclease activity, about 75% decrease in trans nuclease activity to about 99% decrease in trans nuclease activity, about 75% decrease in trans nuclease activity to about 100% decrease in trans nuclease activity, or about 99% decrease in trans nuclease activity to about 100% decrease in trans nuclease activity. A variant RfxCas13d may possess about 1% decrease in trans nuclease activity, about 2% decrease in trans nuclease activity, about 5% decrease in trans nuclease activity, about 10% decrease in trans nuclease activity, about 25% decrease in trans nuclease activity, about 50% decrease in trans nuclease activity, about 75% decrease in trans nuclease activity, about 99% decrease in trans nuclease activity, or about 100% decrease in trans nuclease activity. A variant RfxCas13d may possess at least about 1% decrease in trans nuclease activity, about 2% decrease in trans nuclease activity, about 5% decrease in trans nuclease activity, about 10% decrease in trans nuclease activity, about 25% decrease in trans nuclease activity, about 50% decrease in trans nuclease activity, about 75% decrease in trans nuclease activity, or about 99% decrease in trans nuclease activity. A variant RfxCas13d may possess at most about 2% decrease in trans nuclease activity, about 5% decrease in trans nuclease activity, about 10% decrease in trans nuclease activity, about 25% decrease in trans nuclease activity, about 50% decrease in trans nuclease activity, about 75% decrease in trans nuclease activity, about 99% decrease in trans nuclease activity, or about 100% decrease in trans nuclease activity.

A variant Prevotella sp. P5-125 (PspCas13b) nuclease, wherein said variant PspCas13 nuclease comprises an altered nuclease activity compared to an unaltered PspCas13 in SEQ ID NO: 7. A variant Prevotella sp. P5-125 (PspCas13b) nuclease, wherein said variant PspCas13 nuclease may comprise one or more of the following; an increase in said variant's cis nuclease activity, a decrease in said variant's cis nuclease activity, an increase in said variant's trans nuclease activity, and a decrease in said variant's trans nuclease activity.

The variant Prevotella sp. P'S-125 (PspCas13b) nuclease may comprise one or more amino acid sequence modifications relative to SEQ ID NO: 7. The variant Prevotella sp. P5-125 (PspCas13b) nuclease may comprise one, two, three, four, five, six, seven, eight, nine, ten or more one or more amino acid sequence modifications relative to SEQ ID NO: 7.

The one or more amino acid sequence modifications may be in any one or more PspCas13 domains selected from the list consisting of a HEPN-1 domain, a HEPN-2 domain, a Helical-1 domain, a Helical-2 domain, a Cas13 switch region, a Cas13 nuclease lobe, a Cas13 recognition lobe, a Cas13 linker region, a NTD, a Monolith region, and a Cas13 catalytic site. In certain embodiments, one or more amino acid sequence modifications relative to SEQ ID NO: 7 are in a HEPN nuclease activation domain. In certain embodiments, one or more amino acid sequence modifications relative to SEQ ID NO: 7 are in a RNA binding domain. In certain embodiments, two or more amino acid sequence modifications relative to SEQ ID NO: 7 are in a RNA binding domain and a HEPN nuclease activation domain.

A variant PspCas13 may possess about a 10% increase in cis nuclease activity to about a 1000% increase in cis nuclease activity. A variant PspCas13 may possess about a 10% increase in cis nuclease activity to about a 50% increase in cis nuclease activity, about a 10% increase in cis nuclease activity to about a 100% increase in cis nuclease activity, about a 10% increase in cis nuclease activity to about a 200% increase in cis nuclease activity, about a 10% increase in cis nuclease activity to about a 500% increase in cis nuclease activity, about a 10% increase in cis nuclease activity to about a 1000% increase in cis nuclease activity, about a 50% increase in cis nuclease activity to about a 100% increase in cis nuclease activity, about a 50% increase in cis nuclease activity to about a 200% increase in cis nuclease activity, about a 50% increase in cis nuclease activity to about a 500% increase in cis nuclease activity, about a 50% increase in cis nuclease activity to about a 1000% increase in cis nuclease activity, about a 100% increase in cis nuclease activity to about a 200% increase in cis nuclease activity, about a 100% increase in cis nuclease activity to about a 500% increase in cis nuclease activity, about a 100% increase in cis nuclease activity to about a 1000% increase in cis nuclease activity, about a 200% increase in cis nuclease activity to about a 500% increase in cis nuclease activity, about a 200% increase in cis nuclease activity to about a 1000% increase in cis nuclease activity, or about a 500% increase in cis nuclease activity to about a 1000% increase in cis nuclease activity. A variant PspCas13 may possess about a 10% increase in cis nuclease activity, about a 50% increase in cis nuclease activity, about a 100% increase in cis nuclease activity, about a 200% increase in cis nuclease activity, about a 500% increase in cis nuclease activity, or about a 1000% increase in cis nuclease activity. A variant PspCas13 may possess at least about a 10% increase in cis nuclease activity, about a 50% increase in cis nuclease activity, about a 100% increase in cis nuclease activity, about a 200% increase in cis nuclease activity, or about a 500% increase in cis nuclease activity. A variant PspCas13 may possess at most about a 50% increase in cis nuclease activity, about a 100% increase in cis nuclease activity, about a 200% increase in cis nuclease activity, about a 500% increase in cis nuclease activity, or about a 1000% increase in cis nuclease activity.

A variant PspCas13 may possess about 1% decrease in cis nuclease activity to about 100% decrease in cis nuclease activity. A variant PspCas13 may possess about 1% decrease in cis nuclease activity to about 2% decrease in cis nuclease activity, about 1% decrease in cis nuclease activity to about 5% decrease in cis nuclease activity, about 1% decrease in cis nuclease activity to about 10% decrease in cis nuclease activity, about 1% decrease in cis nuclease activity to about 25% decrease in cis nuclease activity, about 1% decrease in cis nuclease activity to about 50% decrease in cis nuclease activity, about 1% decrease in cis nuclease activity to about 75% decrease in cis nuclease activity, about 1% decrease in cis nuclease activity to about 99% decrease in cis nuclease activity, about 1% decrease in cis nuclease activity to about 100% decrease in cis nuclease activity, about 2% decrease in cis nuclease activity to about 5% decrease in cis nuclease activity, about 2% decrease in cis nuclease activity to about 10% decrease in cis nuclease activity, about 2% decrease in cis nuclease activity to about 25% decrease in cis nuclease activity, about 2% decrease in cis nuclease activity to about 50% decrease in cis nuclease activity, about 2% decrease in cis nuclease activity to about 75% decrease in cis nuclease activity, about 2% decrease in cis nuclease activity to about 99% decrease in cis nuclease activity, about 2% decrease in cis nuclease activity to about 100% decrease in cis nuclease activity, about 5% decrease in cis nuclease activity to about 10% decrease in cis nuclease activity, about 5% decrease in cis nuclease activity to about 25% decrease in cis nuclease activity, about 5% decrease in cis nuclease activity to about 50% decrease in cis nuclease activity, about 5% decrease in cis nuclease activity to about 75% decrease in cis nuclease activity, about 5% decrease in cis nuclease activity to about 99% decrease in cis nuclease activity, about 5% decrease in cis nuclease activity to about 100% decrease in cis nuclease activity, about 10% decrease in cis nuclease activity to about 25% decrease in cis nuclease activity, about 10% decrease in cis nuclease activity to about 50% decrease in cis nuclease activity, about 10% decrease in cis nuclease activity to about 75% decrease in cis nuclease activity, about 10% decrease in cis nuclease activity to about 99% decrease in cis nuclease activity, about 10% decrease in cis nuclease activity to about 100% decrease in cis nuclease activity, about 25% decrease in cis nuclease activity to about 50% decrease in cis nuclease activity, about 25% decrease in cis nuclease activity to about 75% decrease in cis nuclease activity, about 25% decrease in cis nuclease activity to about 99% decrease in cis nuclease activity, about 25% decrease in cis nuclease activity to about 100% decrease in cis nuclease activity, about 50% decrease in cis nuclease activity to about 75% decrease in cis nuclease activity, about 50% decrease in cis nuclease activity to about 99% decrease in cis nuclease activity, about 50% decrease in cis nuclease activity to about 100% decrease in cis nuclease activity, about 75% decrease in cis nuclease activity to about 99% decrease in cis nuclease activity, about 75% decrease in cis nuclease activity to about 100% decrease in cis nuclease activity, or about 99% decrease in cis nuclease activity to about 100% decrease in cis nuclease activity. A variant PspCas13 may possess about 1% decrease in cis nuclease activity, about 2% decrease in cis nuclease activity, about 5% decrease in cis nuclease activity, about 10% decrease in cis nuclease activity, about 25% decrease in cis nuclease activity, about 50% decrease in cis nuclease activity, about 75% decrease in cis nuclease activity, about 99% decrease in cis nuclease activity, or about 100% decrease in cis nuclease activity. A variant PspCas13 may possess at least about 1% decrease in cis nuclease activity, about 2% decrease in cis nuclease activity, about 5% decrease in cis nuclease activity, about 10% decrease in cis nuclease activity, about 25% decrease in cis nuclease activity, about 50% decrease in cis nuclease activity, about 75% decrease in cis nuclease activity, or about 99% decrease in cis nuclease activity. A variant PspCas13 may possess at most about 2% decrease in cis nuclease activity, about 5% decrease in cis nuclease activity, about 10% decrease in cis nuclease activity, about 25% decrease in cis nuclease activity, about 50% decrease in cis nuclease activity, about 75% decrease in cis nuclease activity, about 99% decrease in cis nuclease activity, or about 100% decrease in cis nuclease activity

A variant PspCas13 may possess about a 10% increase in trans nuclease activity to about a 1000% increase in trans nuclease activity. A variant PspCas13 may possess about a 10% increase in trans nuclease activity to about a 50% increase in trans nuclease activity, about a 10% increase in trans nuclease activity to about a 100% increase in trans nuclease activity, about a 10% increase in trans nuclease activity to about a 200% increase in trans nuclease activity, about a 10% increase in trans nuclease activity to about a 500% increase in trans nuclease activity, about a 10% increase in trans nuclease activity to about a 1000% increase in trans nuclease activity, about a 50% increase in trans nuclease activity to about a 100% increase in trans nuclease activity, about a 50% increase in trans nuclease activity to about a 200% increase in trans nuclease activity, about a 50% increase in trans nuclease activity to about a 500% increase in trans nuclease activity, about a 50% increase in trans nuclease activity to about a 1000% increase in trans nuclease activity, about a 100% increase in trans nuclease activity to about a 200% increase in trans nuclease activity, about a 100% increase in trans nuclease activity to about a 500% increase in trans nuclease activity, about a 100% increase in trans nuclease activity to about a 1000% increase in trans nuclease activity, about a 200% increase in trans nuclease activity to about a 500% increase in trans nuclease activity, about a 200% increase in trans nuclease activity to about a 1000% increase in trans nuclease activity, or about a 500% increase in trans nuclease activity to about a 1000% increase in trans nuclease activity. A variant PspCas13 may possess about a 10% increase in trans nuclease activity, about a 50% increase in trans nuclease activity, about a 100% increase in trans nuclease activity, about a 200% increase in trans nuclease activity, about a 500% increase in trans nuclease activity, or about a 1000% increase in trans nuclease activity. A variant PspCas13 may possess at least about a 10% increase in trans nuclease activity, about a 50% increase in trans nuclease activity, about a 100% increase in trans nuclease activity, about a 200% increase in trans nuclease activity, or about a 500% increase in trans nuclease activity. A variant PspCas13 may possess at most about a 50% increase in trans nuclease activity, about a 100% increase in trans nuclease activity, about a 200% increase in trans nuclease activity, about a 500% increase in trans nuclease activity, or about a 1000% increase in trans nuclease activity.

A variant PspCas13 may possess about 1% decrease in trans nuclease activity to about 100% decrease in trans nuclease activity. A variant PspCas13 may possess about 1% decrease in trans nuclease activity to about 2% decrease in trans nuclease activity, about 1% decrease in trans nuclease activity to about 5% decrease in trans nuclease activity, about 1% decrease in trans nuclease activity to about 10% decrease in trans nuclease activity, about 1% decrease in trans nuclease activity to about 25% decrease in trans nuclease activity, about 1% decrease in trans nuclease activity to about 50% decrease in trans nuclease activity, about 1% decrease in trans nuclease activity to about 75% decrease in trans nuclease activity, about 1% decrease in trans nuclease activity to about 99% decrease in trans nuclease activity, about 1% decrease in trans nuclease activity to about 100% decrease in trans nuclease activity, about 2% decrease in trans nuclease activity to about 5% decrease in trans nuclease activity, about 2% decrease in trans nuclease activity to about 10% decrease in trans nuclease activity, about 2% decrease in trans nuclease activity to about 25% decrease in trans nuclease activity, about 2% decrease in trans nuclease activity to about 50% decrease in trans nuclease activity, about 2% decrease in trans nuclease activity to about 75% decrease in trans nuclease activity, about 2% decrease in trans nuclease activity to about 99% decrease in trans nuclease activity, about 2% decrease in trans nuclease activity to about 100% decrease in trans nuclease activity, about 5% decrease in trans nuclease activity to about 10% decrease in trans nuclease activity, about 5% decrease in trans nuclease activity to about 25% decrease in trans nuclease activity, about 5% decrease in trans nuclease activity to about 50% decrease in trans nuclease activity, about 5% decrease in trans nuclease activity to about 75% decrease in trans nuclease activity, about 5% decrease in trans nuclease activity to about 99% decrease in trans nuclease activity, about 5% decrease in trans nuclease activity to about 100% decrease in trans nuclease activity, about 10% decrease in trans nuclease activity to about 25% decrease in trans nuclease activity, about 10% decrease in trans nuclease activity to about 50% decrease in trans nuclease activity, about 10% decrease in trans nuclease activity to about 75% decrease in trans nuclease activity, about 10% decrease in trans nuclease activity to about 99% decrease in trans nuclease activity, about 10% decrease in trans nuclease activity to about 100% decrease in trans nuclease activity, about 25% decrease in trans nuclease activity to about 50% decrease in trans nuclease activity, about 25% decrease in trans nuclease activity to about 75% decrease in trans nuclease activity, about 25% decrease in trans nuclease activity to about 99% decrease in trans nuclease activity, about 25% decrease in trans nuclease activity to about 100% decrease in trans nuclease activity, about 50% decrease in trans nuclease activity to about 75% decrease in trans nuclease activity, about 50% decrease in trans nuclease activity to about 99% decrease in trans nuclease activity, about 50% decrease in trans nuclease activity to about 100% decrease in trans nuclease activity, about 75% decrease in trans nuclease activity to about 99% decrease in trans nuclease activity, about 75% decrease in trans nuclease activity to about 100% decrease in trans nuclease activity, or about 99% decrease in trans nuclease activity to about 100% decrease in trans nuclease activity. A variant PspCas13 may possess about 1% decrease in trans nuclease activity, about 2% decrease in trans nuclease activity, about 5% decrease in trans nuclease activity, about 10% decrease in trans nuclease activity, about 25% decrease in trans nuclease activity, about 50% decrease in trans nuclease activity, about 75% decrease in trans nuclease activity, about 99% decrease in trans nuclease activity, or about 100% decrease in trans nuclease activity. A variant PspCas13 may possess at least about 1% decrease in trans nuclease activity, about 2% decrease in trans nuclease activity, about 5% decrease in trans nuclease activity, about 10% decrease in trans nuclease activity, about 25% decrease in trans nuclease activity, about 50% decrease in trans nuclease activity, about 75% decrease in trans nuclease activity, or about 99% decrease in trans nuclease activity. A variant PspCas13 may possess at most about 2% decrease in trans nuclease activity, about 5% decrease in trans nuclease activity, about 10% decrease in trans nuclease activity, about 25% decrease in trans nuclease activity, about 50% decrease in trans nuclease activity, about 75% decrease in trans nuclease activity, about 99% decrease in trans nuclease activity, or about 100% decrease in trans nuclease activity.

### EXAMPLES

The following illustrative examples are representative of embodiments of compositions and methods described herein and are not meant to be limiting in any way.

### Example 1—a Method to Screen for Cas Nuclease Mutants

Described in this example is a non-limiting embodiment of a method to screen for variant nuclease activity.

### Poisson Distribution Calculation of Droplets

We preserved high single-variant purity by modelling a Poisson loading regime for each step involving microfluidic droplet generation. Using equations for the Poisson distribution and event rate, the concentration of loading material (e.g., beads or plasmid DNA) needed to achieve a low event rate (λ<0.05) was determined. Reagent concentrations were modified accordingly, resulting in mostly empty droplets, <5% of droplets containing a single unit, and almost no droplets containing more than one unit.

### PCR and Construct-to-Bead Coupling in Single Emulsions

Reverse PCR primers for the construct expressing LwaCas13a were immobilized on MyOne Streptavidin CI Dynabeads (Invitrogen). 100 μL of Dynabeads were resuspended in 2× Binding and Washing (B/W) Buffer (10 mM Tris-HCl, 1 mM EDTA, 2 M NaCl) and 5′-biotinylated reverse primer was added to the slurry at 5 uM concentration. The solution was incubated on an end-over-end rotator for 30 minutes at room temperature. Reaction tubes were placed on a magnetic separation rack to isolate the beads from solution, and the beads were washed three times with 1× B/W Buffer. Beads were resuspended in water to the concentration derived from Poisson distribution modelling.

Inner aqueous solutions for droplet generation were prepared as follows. The first solution (‘Inner 1’) comprised 1× PrimeSTAR GXL Buffer, 400 uM/each dNTP, 0.15 uM forward PCR primer, 1.25 U/50 uL PrimeSTAR GXL DNA Polymerase (Takara Bio), and nuclease-free water to the final volume (at least 500 uL). The second solution (‘Inner 2’) comprised LwaCas13a plasmid (Addgene #90097) to a concentration of 0.3 ng/uL, 6% by volume of primer-bearing Dynabeads, and 10% Optiprep (Sigma-Aldrich).

dSURF (Fluigent) was used as the oil solution. Inner and oil solutions were loaded in 1 mL syringes (BD) and 5 mL syringes (BD), respectively. Each syringe was clamped to a Pump 11 Pico Plus Elite syringe pump (Harvard Apparatus). Syringes were connected to the microfluidic devices via polyethylene micro tubing (Scientific Commodities). The droplet generation protocol outlined in Brower et al. was followed with minor modifications. See Brower et al. “Double Emulsion Picoreactors for High-Throughput Single-Cell Encapsulation and Phenotyping via FACS.” Anal Chem. 2020 Oct. 6; 92 (19): 13262-13270. To generate single emulsions, the O2 plasma treatment step was skipped and oil replaced the outer aqueous solution. Typical flow rates were 150 uL/hr for Inner 1, 50 uL/hr for Inner 2, 400 uL/hr for Oil 1, and 300 uL/hr for Oil 2.

Collected droplets were resuspended in the oil carrier phase by gentle agitation and were moved to 0.2 mL PCR tubes. Tubes were placed in a thermocycler and subjected to the following PCR protocol:
1 minute at 98° C.
Repeat 30×:
   10 seconds at 98° C.
   30 seconds at 67° C.
   2.5 minutes at 72° C.
2 minutes at 72° C.

### Preparation of Construct-Coupled Beads for In Vitro Transcription and Translation

Following PCR, droplets were demulsified by adding equal volume of 1H, 1H,2H,2H-Perfluorooctanol to the reaction tube. Tubes were briefly vortexed and placed on an end-over-end rotator for 10 minutes. The separated aqueous phase was carefully extracted and transferred to a different tube. Tubes were placed on a magnetic separation rack and beads were washed three times with 1× B/W buffer, thoroughly removing any residual oil. Beads were resuspended in 2× B/W buffer at their original input volume.

CRISPR RNA (crRNA, chemically synthesized) with a 3′ biotin modification and a spacer sequence designed to target GFP transcript were added to the beads at a final concentration of 0.675 uM. The solution was incubated on an end-over-end rotator for 30 minutes at room temperature, washed three times, and resuspended in water.

### In Vitro Transcription and Translation of Cas13 in Single Emulsions

Inner aqueous solutions for droplet generation were prepared as follows. The first solution (‘Inner 1’) comprised 55% PURExpress Solution A (NEB), 40% PURExpress Solution B (NEB), and 5% rRNasin RNase Inhibitor (Promega). The second solution (‘Inner 2’) comprising the prepared bead suspension. 10% Optiprep (Sigma-Aldrich), and water to a final volume of at least 300 μL. The concentration of the bead suspension was determined in favour of a low event rate. dSURF was used as the oil solution.

As in the previous droplet generation step, the inner solutions flowed in through their respective inlets and oil flowed in through two inlets. Typical flow rates were 100 uL/hr for Inner 1, 100 uL/hr for Inner 2, 400 uL/hr for Oil 1, and 300 uL/hr for Oil 2.

Collected droplets were incubated at 37° C. for 4 hours. Droplets then underwent the same demulsification and wash steps described above.

### Simultaneous Readout of Cis and Trans Cleavage Activity of Cas13

Double emulsion droplets containing CRISPR-Cas13 detection reaction components were generated following the same droplet generation protocol as above, with a few modifications. The first inner solution (‘Inner 1’) comprised PURExpress Solution A (NEB), PURExpress Solution B (NEB), rRNasin RNase Inhibitor (Promega), and fluorophore-quencher (FQ) reporter (custom order from IDT). The second inner solution (‘Inner 2’) comprised crRNA- and Cas13-couple Dynabeads and plasmid DNA encoding GFP (Addgene #29663). dSURF was used as the oil solution. Unlike in the previous steps, an aqueous buffer solution (294 mM HEPES, 1% Tween-20, 2% Pluronix. 25 mM MgSO₄) was used as the outer sheath solution. Following emulsification of reagents, reactions were incubated at 37° C. for 3 hours.

### Example 2—Library of Variant Cas13a Nucleases

As shown in Table 1 below, and FIGS. 6 and 7, a library of variant Cas13a nucleases was generated using an oligonucleotide library and plasmid mutagenesis to create mutations in Leptotrichia wadei Cas13a (LwaCas13a) and Leptotrichia buccalis Cas13a (LbuCas13a) in the NTD, Helical-1 domain, HPN-1 domain. Helical-2 domain. Cas13 linker region, and HPN-2 domain. The mutations in Table 1 are named based on their position in an alignment of the amino acid sequence of LwaCas13a (SEQ ID NO:1) and the amino acid sequence of LbuCas13a (SEQ ID NO:2). The number of mutations per domain in the library of variant Cas13a nucleases as shown in Table 1 are: 53 mutations in NTD; 18 mutations in Helical-1; 7 mutations in HEPN-1; 87 mutations in Helical-2; 20 mutations in the Cas13 linker region; and 32 mutations in HEPN-2.

TABLE 1

| Position in | Position in | Type of | | BLOSUM | Location | Location |
|---|---|---|---|---|---|---|
| Lwa alone | alignment | Mutation | Mutation | .score | (Lobe) | (Domain) |
| 7 | 7 | Mutation | D7G | −3 | REC | NTD |
| 15 | 15 | Mutation | I15T | −2 | REC | NTD |
| 16 | 16 | Mutation | E16S | −1 | REC | NTD |
| 19 | 19 | Mutation | K19R | 3 | REC | NTD |
| 24 | 24 | Mutation | T24E | −2 | REC | NTD |
| 31 | 31 | Mutation | S31D | −1 | REC | NTD |
| 36 | 36 | Mutation | E36A | −2 | REC | NTD |
| 39 | 39 | Mutation | S39N | 1 | REC | NTD |
| 40 | 40 | Mutation | I40M | 2 | REC | NTD |
| 44 | 44 | Mutation | I44M | 2 | REC | NTD |
| 50 | 50 | Mutation | D50S | −1 | REC | NTD |
| 51 | 51 | Mutation | N51S | 1 | REC | NTD |
| 52 | 52 | Mutation | A52T | 0 | REC | NTD |
| 53 | 53 | Mutation | S53E | −1 | REC | NTD |
| 54 | 54 | Mutation | E54T | −2 | REC | NTD |
| 55 | 55 | Mutation | E55K | 1 | REC | NTD |
| 58 | 58 | Mutation | R58Q | 1 | REC | NTD |
| 59 | 59 | Mutation | I59K | −5 | REC | NTD |
| 61 | 61 | Mutation | R61I | −5 | REC | NTD |
| 62 | 62 | Mutation | E62G | −4 | REC | NTD |
| 63 | 63 | Mutation | N63K | 0 | REC | NTD |
| 72 | 72 | Mutation | V72M | 1 | REC | NTD |
| 73 | 73 | Mutation | L73V | 1 | REC | NTD |
| 74 | 74 | Mutation | H74Y | 3 | REC | NTD |
| 78 | 78 | Mutation | S78N | 1 | REC | NTD |
| 79 | 79 | Mutation | V79T | 0 | REC | NTD |
| 81 | 81 | Mutation | Y81S | −3 | REC | NTD |
| 85 | 85 | Mutation | R85G | −4 | REC | NTD |
| 87 | 87 | Mutation | E87K | 1 | REC | NTD |
| 88 | 88 | Mutation | K88E | 1 | REC | NTD |
| 90 | 90 | Mutation | A90I | −3 | REC | NTD |
| 91 | 91 | Deletion | VQ[91- | −8 | REC | NTD |
| | | | 92]del | | | |
| 94 | 94 | Mutation | K94R | 3 | REC | NTD |
| 95 | 95 | Mutation | N95E | −1 | REC | NTD |
| 99 | 99 | Mutation | E99T | −2 | REC | NTD |
| 102 | 102 | Mutation | S102L | −4 | REC | NTD |
| 104 | 104 | Mutation | Y104S | −3 | REC | NTD |
| 106 | 106 | Mutation | L106V | 1 | REC | NTD |
| 107 | 107 | Mutation | K107R | 3 | REC | NTD |
| 108 | 108 | Mutation | N108D | 2 | REC | NTD |
| 110 | 110 | Mutation | N110K | 0 | REC | NTD |
| 111 | 111 | Mutation | S111N | 1 | REC | NTD |
| 113 | 113 | Mutation | S113A | 2 | REC | NTD |
| 119 | 119 | Mutation | L119Y | −2 | REC | NTD |
| 123 | 123 | Mutation | D123N | 2 | REC | NTD |
| 131 | 131 | Mutation | I131V | 4 | REC | NTD |
| 134 | 134 | Mutation | K134N | 0 | REC | NTD |
| 136 | 136 | Mutation | V136I | 4 | REC | NTD |
| 137 | 137 | Mutation | E137K | 1 | REC | NTD |
| 138 | 138 | Mutation | A138K | −1 | REC | NTD |
| 152 | 152 | Mutation | E152K | 1 | REC | NTD |
| 165 | 165 | Mutation | V165I | 4 | REC | NTD |
| 169 | 169 | Mutation | G169E | −4 | REC | NTD |
| 188 | 188 | Mutation | N188D | 2 | REC | Helical-1 |
| 189 | 189 | Mutation | D189A | −3 | REC | Helical-1 |
| 191 | 191 | Mutation | I191V | 4 | REC | Helical-1 |
| 192 | 192 | Mutation | N192S | 1 | REC | Helical-1 |
| 195 | 195 | Mutation | Q195K | 2 | REC | Helical-1 |
| 204 | 204 | Mutation | K204E | 1 | REC | Helical-1 |
| 208 | 208 | Mutation | E208A | −2 | REC | Helical-1 |
| 211 | 211 | Mutation | F211V | −2 | REC | Helical-1 |
| 212 | 212 | Mutation | F212L | 0 | REC | Helical-1 |
| 213 | 213 | Mutation | L213E | −6 | REC | Helical-1 |
| 217 | 217 | Mutation | S217L | −4 | REC | Helical-1 |
| 218 | 218 | Mutation | K218T | −1 | REC | Helical-1 |
| 220 | 220 | Mutation | H220L | −5 | REC | Helical-1 |
| 228 | 228 | Mutation | Y228F | 4 | REC | Helical-1 |
| 231 | 231 | Mutation | K231E | 1 | REC | Helical-1 |
| 256 | 256 | Mutation | I256M | 2 | REC | Helical-1 |
| 263 | 263 | Mutation | I263V | 4 | REC | Helical-1 |
| 359 | 359 | Mutation | V359D | −6 | REC | Helical-1 |
| 403 | 403 | Mutation | G403D | −3 | NUC | HEPN-1 |
| 436 | 436 | Mutation | Q436K | 2 | NUC | HEPN-1 |
| 520 | 520 | Mutation | K520R | 3 | NUC | Helical-2 |
| 529 | 529 | Mutation | N529R | −1 | NUC | Helical-2 |
| 531 | 531 | Mutation | Y531L | −2 | NUC | Helical-2 |
| 534 | 534 | Mutation | D534Y | −6 | NUC | Helical-2 |
| 535 | 535 | Mutation | V535K | −4 | NUC | Helical-2 |
| 537 | 537 | Mutation | I537L | 2 | NUC | Helical-2 |
| 538 | 538 | Mutation | K538N | 0 | NUC | Helical-2 |
| 542 | 542 | Mutation | N542R | −1 | NUC | Helical-2 |
| 544 | 544 | Mutation | K544R | 3 | NUC | Helical-2 |
| 546 | 546 | Mutation | N546E | −1 | NUC | Helical-2 |
| 563 | 563 | Mutation | N563S | 1 | NUC | Helical-2 |
| 564 | 564 | Mutation | K564R | 3 | NUC | Helical-2 |
| 566 | 566 | Mutation | E566D | 2 | NUC | Helical-2 |
| 569 | 569 | Mutation | R569K | 3 | NUC | Helical-2 |
| 571 | 571 | Mutation | T571S | 2 | NUC | Helical-2 |
| 573 | 573 | Mutation | K573G | −3 | NUC | Helical-2 |
| 574 | 574 | Mutation | F574I | −1 | NUC | Helical-2 |
| 575 | 575 | Mutation | F575Y | 4 | NUC | Helical-2 |
| 577 | 577 | Mutation | S577K | −1 | NUC | Helical-2 |
| 578 | 578 | Mutation | V578T | 0 | NUC | Helical-2 |
| 581 | 581 | Mutation | D581T | −2 | NUC | Helical-2 |
| 582 | 582 | Mutation | K582N | 0 | NUC | Helical-2 |
| 583 | 583 | Mutation | E583D | 2 | NUC | Helical-2 |
| 584 | 584 | Mutation | E584D | 2 | NUC | Helical-2 |
| 585 | 585 | Mutation | K585N | 0 | NUC | Helical-2 |
| 585+ | 586-591 | Insertion | Ins[586- | −8 | NUC | Helical-2 |
| | | | 91]KTKEII | | | |
| 603 | 609 | Mutation | K609Y | −4 | NUC | Helical-2 |
| 605 | 611 | Mutation | V611M | 1 | NUC | Helical-2 |
| 606 | 612 | Mutation | K612S | −1 | NUC | Helical-2 |
| 608 | 614 | Mutation | S614N | 1 | NUC | Helical-2 |
| 609 | 615 | Mutation | K615G | −3 | NUC | Helical-2 |
| 610 | 616 | Mutation | V616N | −5 | NUC | Helical-2 |
| 613 | 619 | Mutation | K619E | 1 | NUC | Helical-2 |
| 615 | 621 | Mutation | T621S | 2 | NUC | Helical-2 |
| 616 | 622 | Mutation | N622K | 0 | NUC | Helical-2 |
| 618 | 624 | Mutation | V624I | 4 | NUC | Helical-2 |
| 620 | 626 | Mutation | K626E | 1 | NUC | Helical-2 |
| 621 | 627 | Mutation | I627L | 2 | NUC | Helical-2 |
| 623+ | 630-631 | Insertion | Ins[630- | −8 | NUC | Helical-2 |
| | | | 631]ND | | | |
| 624 | 632 | Mutation | Q632K | 2 | NUC | Helical-2 |
| 627 | 635 | Mutation | Q635L | −4 | NUC | Helical-2 |
| 631 | 639 | Mutation | H639F | −2 | NUC | Helical-2 |
| 634 | 642 | Mutation | Y642L | −2 | NUC | Helical-2 |
| 639 | 647 | Mutation | N647D | 2 | NUC | Helical-2 |
| 641 | 649 | Mutation | E649Q | 3 | NUC | Helical-2 |
| 642 | 650 | Mutation | K650E | 1 | NUC | Helical-2 |
| 643 | 651 | Mutation | T651K | −1 | NUC | Helical-2 |
| 644 | 652 | Mutation | V652I | 4 | NUC | Helical-2 |
| 646 | 654 | Mutation | V654K | −4 | NUC | Helical-2 |
| 651 | 659 | Mutation | I659N | −6 | NUC | Helical-2 |
| 655 | 663 | Mutation | R663L | −4 | NUC | Helical-2 |
| 656 | 664 | Mutation | E664Y | −5 | NUC | Helical-2 |
| 660+ | 668-669 | Insertion | Ins668- | −8 | NUC | Helical-2 |
| | | | 669AG | | | |
| 663 | 673 | Mutation | K673E | 1 | NUC | Helical-2 |
| 667 | 677 | Mutation | N677D | 2 | NUC | Helical-2 |
| 675 | 685 | Mutation | Q685K | 2 | NUC | Helical-2 |
| 682 | 692 | Mutation | I692M | 2 | NUC | Helical-2 |
| 683 | 693 | Mutation | D693T | −2 | NUC | Helical-2 |
| 686 | 696 | Mutation | N696A | −3 | NUC | Helical-2 |
| 687 | 697 | Mutation | K697N | 0 | NUC | Helical-2 |
| 688+ | 699-701 | Insertion | Ins[699- | −8 | NUC | Helical-2 |
| | | | 701]GRL | | | |
| 689 | 702 | Mutation | N702S | 1 | NUC | Helical-2 |
| 691 | 704 | Mutation | K704I | −5 | NUC | Helical-2 |
| 694 | 707 | Mutation | E707G | −4 | NUC | Helical-2 |
| 696 | 709 | Mutation | N709D | 2 | NUC | Helical-2 |
| 697 | 710 | Mutation | N710E | −1 | NUC | Helical-2 |
| 698 | 711 | Mutation | N711E | −1 | NUC | Helical-2 |
| 699 | 712 | Mutation | N712T | 0 | NUC | Helical-2 |
| 700 | 713 | Mutation | D713N | 2 | NUC | Helical-2 |
| 701 | 714 | Mutation | N714T | 0 | NUC | Helical-2 |
| 702 | 715 | Mutation | N715S | 1 | NUC | Helical-2 |
| 703 | 716-723 | Deletion | DIFSKIKI[716- | −8 | NUC | Helical-2 |
| | | | 723]del | | | |
| 711 | 724 | Mutation | K724L | −4 | NUC | Helical-2 |
| 712 | 725 | Mutation | K725A | −1 | NUC | Helical-2 |
| 713 | 726 | Mutation | D726E | 2 | NUC | Helical-2 |
| 714 | 727 | Mutation | N727K | 0 | NUC | Helical-2 |
| 716 | 729 | Mutation | E729Q | 3 | NUC | Helical-2 |
| 717 | 730 | Mutation | K730E | 1 | NUC | Helical-2 |
| 718 | 731 | Mutation | Y731F | 4 | NUC | Helical-2 |
| 721 | 734 | Mutation | I734F | −1 | NUC | Helical-2 |
| 724 | 737 | Mutation | N737K | 0 | NUC | Helical-2 |
| 727 | 740 | Mutation | K740Q | 2 | NUC | Helical-2 |
| 728 | 741 | Mutation | H741N | 1 | NUC | Helical-2 |
| 730 | 743 | Deletion | R743del | −8 | NUC | Helical-2 |
| 732 | 745 | Mutation | K745I | −5 | NUC | Helical-2 |
| 733 | 746 | Mutation | E746K | 1 | NUC | Helical-2 |
| 736 | 749 | Mutation | H749Y | 3 | NUC | Helical-2 |
| 742 | 755 | Mutation | V755L | 1 | NUC | HEPN-1 |
| 749 | 762 | Mutation | K762N | 0 | NUC | HEPN-1 |
| 756 | 769 | Mutation | N769R | −1 | NUC | HEPN-1 |
| 790 | 803 | Mutation | T803A | 0 | NUC | HEPN-1 |
| 794 | 807 | Mutation | E807Q | 3 | NUC | HEPN-1 |
| 817 | 830 | Mutation | N830D | 2 | NUC | Linker |
| 827 | 840 | Mutation | E840G | −4 | NUC | Linker |
| 830 | 843 | Mutation | I843V | 4 | NUC | Linker |
| 833 | 846 | Mutation | R846N | −1 | NUC | Linker |
| 877 | 890 | Mutation | K890G | −3 | NUC | Linker |
| 882 | 895 | Mutation | L895I | 2 | NUC | Linker |
| 883 | 896 | Mutation | K896E | 1 | NUC | Linker |
| 887 | 900 | Mutation | E900K | 1 | NUC | Linker |
| 899 | 912 | Mutation | Y912H | 3 | NUC | Linker |
| 900 | 913 | Mutation | T913K | −1 | NUC | Linker |
| 903 | 916 | Mutation | Q916E | 3 | NUC | Linker |
| 913 | 926 | Mutation | K926R | 3 | NUC | Linker |
| 919 | 932 | Mutation | N932T | 0 | NUC | Linker |
| 924 | 937 | Mutation | K937E | 1 | NUC | Linker |
| 925 | 938 | Mutation | E938S | −1 | NUC | Linker |
| 927 | 940 | Mutation | E940K | 1 | NUC | Linker |
| 928 | 941 | Mutation | K941Q | 2 | NUC | Linker |
| 931 | 944 | Mutation | G944E | −4 | NUC | Linker |
| 934 | 947 | Mutation | Q947E | 3 | NUC | Linker |
| 935 | 948 | Mutation | K948E | 1 | NUC | Linker |
| 957 | 970 | Mutation | K970R | 3 | NUC | HEPN-2 |
| 985 | 998 | Mutation | H998Q | 1 | NUC | HEPN-2 |
| 994 | 1007 | Mutation | D1007E | 2 | NUC | HEPN-2 |
| 996 | 1009 | Mutation | S1009K | −1 | NUC | HEPN-2 |
| 1003 | 1016 | Mutation | S1016G | −1 | NUC | HEPN-2 |
| 1012 | 1025 | Mutation | N1025K | 0 | NUC | HEPN-2 |
| 1018 | 1031 | Mutation | Y1031H | 3 | NUC | HEPN-2 |
| 1019 | 1032 | Mutation | K1032Q | 2 | NUC | HEPN-2 |
| 1020 | 1033 | Mutation | D1033N | 2 | NUC | HEPN-2 |
| 1021 | 1034 | Mutation | N1034D | 2 | NUC | HEPN-2 |
| 1022 | 1035 | Mutation | V1035E | −4 | NUC | HEPN-2 |
| 1023 | 1036 | Mutation | E1036V | −4 | NUC | HEPN-2 |
| 1025 | 1038 | Mutation | R1038I | −5 | NUC | HEPN-2 |
| 1026 | 1039 | Mutation | S1039N | 1 | NUC | HEPN-2 |
| 1027 | 1040 | Mutation | I1040K | −5 | NUC | HEPN-2 |
| 1030 | 1043 | Mutation | D1043S | −1 | NUC | HEPN-2 |
| 1031 | 1044 | Mutation | K1044A | −1 | NUC | HEPN-2 |
| 1032 | 1045 | Mutation | K1045N | 0 | NUC | HEPN-2 |
| 1033 | 1046 | Mutation | V1046I | 4 | NUC | HEPN-2 |
| 1035 | 1048 | Mutation | K1048V | −4 | NUC | HEPN-2 |
| 1083 | 1096 | Mutation | I1096V | 4 | NUC | HEPN-2 |
| 1087 | 1100 | Mutation | I1100V | 4 | NUC | HEPN-2 |
| 1109 | 1122 | Mutation | E1122G | −4 | NUC | HEPN-2 |
| 1140 | 1153 | Mutation | E1153K | 1 | NUC | HEPN-2 |
| 1144 | 1157 | Mutation | V1157I | 4 | NUC | HEPN-2 |
| 1150 | 1163-1168 | Deletion | ALEAAA[1163- | −8 | NUC | HEPN-2 |
| | | | 1168]del | | | |
| 1156 | 1169 | Mutation | L1169M | 3 | NUC | HEPN-2 |
| 1158 | 1171 | Mutation | A1171E | −2 | NUC | HEPN-2 |
| 1159 | 1172 | Mutation | R1172K | 3 | NUC | HEPN-2 |
| 1161 | 1174-1181 | Deletion | EAELAAAT[1174- | −8 | NUC | HEPN-2 |
| | | | 1181]del | | | |
| 1169 | 1182 | Mutation | A1182S | 2 | NUC | HEPN-2 |
| 1171 | 1184 | Mutation | Q1184N | 0 | NUC | HEPN-2 |

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

Sequence listings provided herein

| SEQ | | |
|---|---|---|
| ID | | |
| NO: | Sequence | Origin |
| 1 | MKVTKVDGISHKKYIEEGKLVKSTSEENRTSERLSELLSIRLDIYIKNPD | LwaCas13a |
| | NASEEENRIRRENLKKFFSNKVLHLKDSVLYLKNRKEKNAVQDKNYS | amino acid |
| | EEDISEYDLKNKNSFSVLKKILLNEDVNSEELEIFRKDVEAKLNKINSLK | sequence |
| | YSFEENKANYQKINENNVEKVGGKSKRNIIYDYYRESAKRNDYINNVQ | |
| | EAFDKLYKKEDIEKLFFLIENSKKHEKYKIREYYHKIIGRKNDKENFAKI | |
| | IYEEIQNVNNIKELIEKIPDMSELKKSQVFYKYYLDKEELNDKNIKYAF | |
| | CHFVEIEMSQLLKNYVYKRLSNISNDKIKRIFEYQNLKKLIENKLLNKL | |
| | DTYVRNCGKYNYYLQVGEIATSDFIARNRQNEAFLRNIIGVSSVAYFSL | |
| | RNILETENENDITGRMRGKTVKNNKGEEKYVSGEVDKIYNENKQNEV | |
| | KENLKMFYSYDFNMDNKNEIEDFFANIDEAISSIRHGIVHFNLELEGKDI | |
| | FAFKNIAPSEISKKMFQNEINEKKLKLKIFKQLNSANVFNYYEKDVIIKY | |
| | LKNTKFNFVNKNIPFVPSFTKLYNKIEDLRNTLKFFWSVPKDKEEKDA | |
| | QIYLLKNIYYGEFLNKFVKNSKVFFKITNEVIKINKQRNQKTGHYKYQ | |
| | KFENIEKTVPVEYLAIIQSREMINNQDKEEKNTYIDFIQQIFLKGFIDYLN | |
| | KNNLKYIESNNNNDNNDIFSKIKIKKDNKEKYDKILKNYEKHNRNKEIP | |
| | HEINEFVREIKLGKILKYTENLNMFYLILKLLNHKELTNLKGSLEKYQS | |
| | ANKEETFSDELELINLLNLDNNRVTEDFELEANEIGKFLDFNENKIKDR | |
| | KELKKFDTNKIYFDGENIIKHRAFYNIKKYGMLNLLEKIADKAKYKISL | |
| | KELKEYSNKKNEIEKNYTMQQNLHRKYARPKKDEKFNDEDYKEYEK | |
| | AIGNIQKYTHLKNKVEFNELNLLQGLLLKILHRLVGYTSIWERDLRFRL | |
| | KGEFPENHYIEEIFNFDNSKNVKYKSGQIVEKYINFYKELYKDNVEKRS | |
| | IYSDKKVKKLKQEKKDLYIRNYIAHFNYIPHAEISLLEVLENLRKLLSY | |
| | DRKLKNAIMKSIVDILKEYGFVATFKIGADKKIEIQTLESEKIVHLKNLK | |
| 2 | KKKLMTDRNSEELCELVKVMFEYKALE | |
| | atgaaagtgaccaaggtcgacggcatcagccacaagaagtacatcgaagagggcaagctcgtgaagtccacc | LwaCas13a |
| | agcgaggaaaaccggaccagcgagagactgagcgagctgctgagcatccggctggacatctacatcaagaa | nucleic |
| | ccccgacaacgcctccgaggaagagaaccggatcagaagagagaacctgaagaagttctttagcaacaaggt | acid |
| | gctgcacctgaaggacagcgtgctgtatctgaagaaccggaaagaaaagaacgccgtgcaggacaagaacta | sequence |
| | tagcgaagaggacatcagcgagtacgacctgaaaaacaagaacagcttctccgtgctgaagaagatcctgctg | |
| | aacgaggacgtgaactctgaggaactggaaatctttcggaaggacgtggaagccaagctgaacaagatcaac | |
| | agcctgaagtacagcttcgaagagaacaaggccaactaccagaagatcaacgagaacaacgtggaaaaagtg | |
| | ggcggcaagagcaagcggaacatcatctacgactactacagagagagcgccaagcgcaacgactacatcaa | |
| | caacgtgcaggaagccttcgacaagctgtataagaaagaggatatcgagaaactgtttttcctgatcgagaacag | |
| | caagaagcacgagaagtacaagatccgcgagtactatcacaagatcatcggccggaagaacgacaaagaga | |
| | acttcgccaagattatctacgaagagatccagaacgtgaacaacatcaaagagctgattgagaagatccccgac | |
| | atgtctgagctgaagaaaagccaggtgttctacaagtactacctggacaaagaggaactgaacgacaagaatat | |
| | taagtacgccttctgccacttcgtggaaatcgagatgtcccagctgctgaaaaactacgtgtacaagcggctgag | |
| | caacatcagcaacgataagatcaagcggatcttcgagtaccagaatctgaaaaagctgatcgaaaacaaactgc | |
| | tgaacaagctggacacctacgtgcggaactgcggcaagtacaactactatctgcaagtgggcgagatcgccac | |
| | ctccgactttatcgcccggaaccggcagaacgaggccttcctgagaaacatcatcggcgtgtccagcgtggcct | |
| | acttcagcctgaggaacatcctggaaaccgagaacgagaacgatatcaccggccggatgcggggcaagacc | |
| | gtgaagaacaacaagggcgaagagaaatacgtgtccggcgaggtggacaagatctacaatgagaacaagca | |
| | gaacgaagtgaaagaaaatctgaagatgttctacagctacgacttcaacatggacaacaagaacgagatcgag | |
| | gacttcttcgccaacatcgacgaggccatcagcagcatcagacacggcatcgtgcacttcaacctggaactgga | |
| | aggcaaggacatcttcgccttcaagaatatcgcccccagcgagatctccaagaagatgtttcagaacgaaatca | |
| | acgaaaagaagctgaagctgaaaatcttcaagcagctgaacagcgccaacgtgttcaactactacgagaagga | |
| | tgtgatcatcaagtacctgaagaataccaagttcaacttcgtgaacaaaaacatccccttcgtgcccagcttcacc | |
| | aagctgtacaacaagattgaggacctgcggaataccctgaagtttttttggagcgtgcccaaggacaaagaaga | |
| | gaaggacgcccagatctacctgctgaagaatatctactacggcgagttcctgaacaagttcgtgaaaaactccaa | |
| | ggtgttctttaagatcaccaatgaagtgatcaagattaacaagcagcggaaccagaaaaccggccactacaagt | |
| | atcagaagttcgagaacatcgagaaaaccgtgcccgtggaatacctggccatcatccagagcagagagatgat | |
| | caacaaccaggacaaagaggaaaagaatacctacatcgactttattcagcagattttcctgaagggcttcatcga | |
| | ctacctgaacaagaacaatctgaagtatatcgagagcaacaacaacaatgacaacaacgacatcttctccaagat | |
| | caagatcaaaaaggataacaaagagaagtacgacaagatcctgaagaactatgagaagcacaatcggaacaa | |
| | agaaatccctcacgagatcaatgagttcgtgcgcgagatcaagctggggaagattctgaagtacaccgagaatc | |
| | tgaacatgttttacctgatcctgaagctgctgaaccacaaagagctgaccaacctgaagggcagcctggaaaag | |
| | taccagtccgccaacaaagaagaaaccttcagcgacgagctggaactgatcaacctgctgaacctggacaaca | |
| | acagagtgaccgaggacttcgagctggaagccaacgagatcggcaagttcctggacttcaacgaaaacaaaat | |
| | caaggaccggaaagagctgaaaaagttcgacaccaacaagatctatttcgacggcgagaacatcatcaagcac | |
| | cgggccttctacaatatcaagaaatacggcatgctgaatctgctggaaaagatcgccgataaggccaagtataa | |
| | gatcagcctgaaagaactgaaagagtacagcaacaagaagaatgagattgaaaagaactacaccatgcagca | |
| | gaacctgcaccggaagtacgccagacccaagaaggacgaaaagttcaacgacgaggactacaaagagtatg | |
| | agaaggccatcggcaacatccagaagtacacccacctgaagaacaaggtggaattcaatgagctgaacctgct | |
| | gcagggcctgctgctgaagatcctgcaccggctcgtgggctacaccagcatctgggagcgggacctgagattc | |
| | cggctgaagggcgagtttcccgagaaccactacatcgaggaaattttcaatttcgacaactccaagaatgtgaag | |
| | tacaaaagcggccagatcgtggaaaagtatatcaacttctacaaagaactgtacaaggacaatgtggaaaagcg | |
| | gagcatctactccgacaagaaagtgaagaaactgaagcaggaaaaaaaggacctgtacatccggaactacatt | |
| | gcccacttcaactacatcccccacgccgagattagcctgctggaagtgctggaaaacctgcggaagctgctgtc | |
| | ctacgaccggaagctgaagaacgccatcatgaagtccatcgtggacattctgaaagaatacggcttcgtggcca | |
| | ccttcaagatcggcgctgacaagaagatcgaaatccagaccctggaatcagagaagatcgtgcacctgaagaa | |
| | tctgaagaaaaagaaactgatgaccgaccggaacagcgaggaactgtgcgaactcgtgaaagtcatgttcgag | |
| | tacaaggccctggaataa | |
| 3 | MKVTKVGGISHKKYTSEGRLVKSESEENRTDERLSALLNMRLDMYIK | LbuCas13a |
| | NPSSTETKENQKRIGKLKKFFSNKMVYLKDNTLSLKNGKKENIDREYS | amino acid |
| | ETDILESDVRDKKNFAVLKKIYLNENVNSEELEVFRNDIKKKLNKINSL | sequence |
| | KYSFEKNKANYQKINENNIEKVEGKSKRNIIYDYYRESAKRDAYVSNV | |
| | KEAFDKLYKEEDIAKLVLEIENLTKLEKYKIREFYHEIIGRKNDKENFA | |
| | KIIYEEIQNVNNMKELIEKVPDMSELKKSQVFYKYYLDKEELNDKNIK | |
| | YAFCHFVEIEMSQLLKNYVYKRLSNISNDKIKRIFEYQNLKKLIENKLL | |
| | NKLDTYVRNCGKYNYYLQDGEIATSDFIARNRQNEAFLRNIIGVSSVA | |
| | YFSLRNILETENENDITGRMRGKTVKNNKGEEKYVSGEVDKIYNENKK | |
| | NEVKENLKMFYSYDFNMDNKNEIEDFFANIDEAISSIRHGIVHFNLELE | |
| | GKDIFAFKNIAPSEISKKMFQNEINEKKLKLKIFRQLNSANVFRYLEKY | |
| | KILNYLKRTRFEFVNKNIPFVPSFTKLYSRIDDLKNSLGIYWKTPKTND | |
| | DNKTKEIIDAQIYLLKNIYYGEFLNYFMSNNGNFFEISKEIIELNKNDKR | |
| | NLKTGFYKLQKFEDIQEKIPKEYLANIQSLYMINAGNQDEEEKDTYIDF | |
| | IQKIFLKGFMTYLANNGRLSLIYIGSDEETNTSLAEKKQEFDKFLKKYE | |
| | QNNNIKIPYEINEFLREIKLGNILKYTERLNMFYLILKLLNHKELTNLKG | |
| | SLEKYQSANKEEAFSDQLELINLLNLDNNRVTEDFELEADEIGKFLDFN | |
| | GNKVKDNKELKKFDTNKIYFDGENIIKHRAFYNIKKYGMLNLLEKIAD | |
| | KAGYKISIEELKKYSNKKNEIEKNHKMQENLHRKYARPRKDEKFTDED | |
| | YESYKQAIENIEEYTHLKNKVEFNELNLLQGLLLRILHRLVGYTSIWER | |
| | DLRFRLKGEFPENQYIEEIFNFENKKNVKYKGGQIVEKYIKFYKELHQN | |
| | DEVKINKYSSANIKVLKQEKKDLYIRNYIAHFNYIPHAEISLLEVLENLR | |
| | KLLSYDRKLKNAVMKSVVDILKEYGFVATFKIGADKKIGIQTLESEKIV | |
| | HLKNLKKKKLMTDRNSEELCKLVKIMFEYKMEEKKSEN | |
| 4 | atgaaagttacaaaagttggaggaatttcacataaaaaatatactagtgaaggaaggcttgtaaaaagcgaaagt | LbuCas13a |
| | gaagaaaataggactgatgagagattatctgcacttttaaatatgagactagatatgtatataaaaaatcctagcag | nucleic |
| | tacagaaacaaaagaaaatcaaaaaagaataggaaaactgaaaaaattcttttcaaataaaatggtgtatttaaaa | acid |
| | gacaataccttaagcttaaagaatggaaaaaaggaaaatattgacagggaatattctgaaacagatattttggaat | sequence |
| | ccgatgtaagagataaaaaaaattttgcggttttaaaaaaaatatatttaaatgaaaatgtaaattcagaagaattgg | |
| | aagtatttagaaacgatattaagaaaaaattgaataaaataaattctttaaaatattcatttgaaaaaaataaggctaa | |
| | ttaccagaaaattaatgaaaataatattgaaaaagttgaaggaaagagcaaaagaaatattatttatgattattacag | |
| | agaatcagcaaaacgtgatgcttatgtgagtaatgtaaaagaagcctttgataaattatataaagaagaggatattg | |
| | ccaagttagttttagaaatagaaaatttaacaaagcttgaaaaatataaaataagagaattttatcacgaaataattg | |
| | gaagaaaaaatgataaagaaaattttgctaaaattatttatgaagaaatacaaaatgtaaataatatgaaagaattaa | |
| | ttgaaaaagttccagatatgagtgaattaaaaaaatcacaagtgttttataaatattatttggataaagaagaacttaa | |
| | tgataaaaatataaaatatgctttttgtcattttgtggaaattgaaatgagtcagcttttgaaaaattatgtgtataaa | |
| | agactgagtaatataagtaacgataaaattaagagaatatttgaatatcaaaatttaaaaaaattaattgaaaataaat | |
| | tactaaataaattggatacttatgtaagaaattgcgggaaatataattattatttacaagatggagaaattgcaacaag | |
| | tgattttattgctaggaatagacaaaatgaagcatttttacgaaatataattggagtttcttcggttgcatatttttca | |
| | ttgagaaatattcttgaaactgaaaatgagaatgatattacaggtagaatgagaggaaagactgtaaaaaataataag | |
| | ggcgaagaaaaatatgtttctggagaagttgataaaatatacaatgaaaacaagaaaaatgaagtaaaagaaaat | |
| | ttaaaaatgttctatagttatgattttaatatggataataaaaatgagatagaagattttttcgcaaatattgatgaag | |
| | ctattagcagtattagacatgggattgtgcattttaatttggaattagaagggaaagatatatttgcatttaaaaatat | |
| | agctccttctgaaatttcaaaaaaaatgtttcaaaatgaaataaatgagaaaaaattgaaattgaagatatttaggcag | |
| | ttgaatagtgcgaatgtatttaggtatttggaaaaatataaaatattaaattatttaaagagaacacgatttgagtttg | |
| | ttaataaaaatattccatttgttccgtcatttacaaaattgtacagcaggatagatgatttaaaaaatagtctaggtat | |
| | ttattggaaaactccgaaaacaaatgatgataataaaactaaagaaattatagatgctcagatatatcttttgaaaaat | |
| | atttattatggagaatttttaaattattttatgagcaataacgggaatttttttgagataagcaaagaaataattgaat | |
| | taaataaaaatgataaaagaaatttaaaaactggattttataaactacaaaaatttgaagatattcaagaaaaaattcc | |
| | taaagaatatcttgcaaatatacaaagcctttatatgattaatgctggaaatcaggatgaagaggaaaaagatacatat | |
| | attgactttatacaaaaaatatttttaaaaggatttatgacttacttggcaaataacggaagattaagcctaatatata | |
| | ttggaagcgatgaagaaacaaatacttctttagcagaaaaaaagcaagaatttgataaattcttgaaaaaatatgaaca | |
| | aaataataatattaaaattccatatgaaataaatgaatttttaagagagataaaattaggaaatatattaaaatacacc | |
| | gaaagattgaacatgttttatttaattttaaaattgcttaatcataaagaattgactaatttgaaaggaagtcttgaaa | |
| | aatatcagagtgcaaataaagaagaagctttttcagatcaacttgaacttataaatcttttaaatttagataataatag | |
| | agtaacagaagattttgaattagaagcggatgaaattgggaagtttttagattttaatggaaataaggtaaaagataat | |
| | aaagaattgaagaaatttgacacaaataaaatatattttgatggagaaaatattataaagcatagagctttttataata | |
| | taaaaaaatatgggatgttaaatttacttgagaaaatagcggataaggcaggatataaaataagtatagaagaattg | |
| | aaaaaatacagtaataaaaaaaatgaaattgaaaaaaatcataaaatgcaggaaaatttacatagaaaatatgcaa | |
| | gacctagaaaagatgaaaaatttacagatgaagattatgaaagctataaacaggcaattgaaaatatagaagaat | |
| | atactcatttgaaaaataaagtagaatttaatgagttaaatttgttacaaggtctactattaagaatacttcatagact | |
| | tgtggggtatacttcgatttgggaaagagatttgagatttagattaaaaggggaatttccagaaaatcagtatataga | |
| | agaaatatttaattttgagaataaaaagaatgtgaaatataaaggtggacaaattgttgaaaagtatataaaattttat | |
| | aaggaattacatcaaaatgatgaagtaaaaataaataaatattccagtgcaaatataaaagtgttaaaacaagaga | |
| | aaaaagatttgtatatacgaaactatattgcacatttcaactatattccgcatgctgaaatttcacttttagaagtgct | |
| | ggaaaatttaagaaaactactttcttatgatagaaaacttaaaaatgcagttatgaaatcagtagtggatatattaaag | |
| | gaatatggttttgtagcaacatttaaaataggagcagataagaaaataggaattcagactttagagtcggaaaaaa | |
| | ttgtacatttgaaaaatttaaaaaagaaaaaattaatgactgatagaaattcagaagaattgtgtaaacttgtgaaga | |
| | ttatgtttgaatataaaatggaagaaaaaaaatctgaaaactaa | |
| 5 | MIEKKKSFAKGMGVKSTLVSGSKVYMTTFAEGSDARLEKIVEGDSIRS | RfxCas13d |
| | VNEGEAFSAEMADKNAGYKIGNAKFSHPKGYAVVANNPLYTGPVQQ | (CasRx) |
| | DMLGLKETLEKRYFGESADGNDNICIQVIHNILDIEKILAEYITNAAYA | amino acid |
| | VNNISGLDKDIIGFGKFSTVYTYDEFKDPEHHRAAFNNNDKLINAIKAQ | sequence |
| | YDEFDNFLDNPRLGYFGQAFFSKEGRNYIINYGNECYDILALLSGLRH | |
| | WVVHNNEEESRISRTWLYNLDKNLDNEYISTLNYLYDRITNELTNSFS | |
| | KNSAANVNYIAETLGINPAEFAEQYFRFSIMKEQKNLGFNITKLREVML | |
| | DRKDMSEIRKNHKVFDSIRTKVYTMMDFVIYRYYIEEDAKVAAANKS | |
| | LPDNEKSLSEKDIFVINLRGSFNDDQKDALYYDEANRIWRKLENIMHNI | |
| | KEFRGNKTREYKKKDAPRLPRILPAGRDVSAFSKLMYALTMFLDGKEI | |
| | NDLLTTLINKFDNIQSFLKVMPLIGVNAKFVEEYAFFKDSAKIADELRLI | |
| | KSFARMGEPIADARRAMYIDAIRILGTNLSYDELKALADTFSLDENGN | |
| | KLKKGKHGMRNFIINNVISNKRFHYLIRYGDPAHLHEIAKNEAVVKFV | |
| | LGRIADIQKKQGQNGKNQIDRYYETCIGKDKGKSVSEKVDALTKIITG | |
| | MNYDQFDKKRSVIEDTGRENAEREKFKKIISLYLTVIYHILKNIVNINAR | |
| | YVIGFHCVERDAQLYKEKGYDINLKKLEEKGFSSVTKLCAGIDETAPD | |
| | KRKDVEKEMAERAKESIDSLESANPKLYANYIKYSDEKKAEEFTRQIN | |
| | REKAKTALNAYLRNTKWNVIIREDLLRIDNKTCTLFRNKAVHLEVARY | |
| | VHAYINDIAEVNSYFQLYHYIMQRIIMNERYEKSSGKVSEYFDAVNDE | |
| | KKYNDRLLKLLCVPFGYCIPRFKNLSIEALFDRNEAAKFDKEKKKVSG | |
| | NS | |
| 6 | atcgaaaaaaaaaagtccttcgccaagggcatgggcgtgaagtccacactcgtgtccggctccaaagtgtacat | RfxCas13d |
| | gacaaccttcgccgaaggcagcgacgccaggctggaaaagatcgtggagggcgacagcatcaggagcgtg | (CasRx) |
| | aatgagggcgaggccttcagcgctgaaatggccgataaaaacgccggctataagatcggcaacgccaaattca | nucleic |
| | gccatcctaagggctacgccgtggtggctaacaaccctctgtatacaggacccgtccagcaggatatgctcggc | acid |
| | ctgaaggaaactctggaaaagaggtacttcggcgagagcgctgatggcaatgacaatatttgtatccaggtgatc | sequence |
| | cataacatcctggacattgaaaaaatcctcgccgaatacattaccaacgccgcctacgccgtcaacaatatctcc | |
| | ggcctggataaggacattattggattcggcaagttctccacagtgtatacctacgacgaattcaaagaccccgag | |
| | caccatagggccgctttcaacaataacgataagctcatcaacgccatcaaggcccagtatgacgagttcgacaa | |
| | cttcctcgataaccccagactcggctatttcggccaggcctttttcagcaaggagggcagaaattacatcatcaatt | |
| | acggcaacgaatgctatgacattctggccctcctgagcggactgaggcactgggtggtccataacaacgaaga | |
| | agagtccaggatctccaggacctggctctacaacctcgataagaacctcgacaacgaatacatctccaccctca | |
| | actacctctacgacaggatcaccaatgagctgaccaactccttctccaagaactccgccgccaacgtgaactata | |
| | ttgccgaaactctgggaatcaaccctgccgaattcgccgaacaatatttcagattcagcattatgaaagagcaga | |
| | aaaacctcggattcaatatcaccaagctcagggaagtgatgctggacaggaaggatatgtccgagatcaggaa | |
| | aaatcataaggtgttcgactccatcaggaccaaggtctacaccatgatggactttgtgatttataggtattacatcga | |
| | agaggatgccaaggtggctgccgccaataagtccctccccgataatgagaagtccctgagcgagaaggatatc | |
| | tttgtgattaacctgaggggctccttcaacgacgaccagaaggatgccctctactacgatgaagctaatagaattt | |
| | ggagaaagctcgaaaatatcatgcacaacatcaaggaatttaggggaaacaagacaagagagtataagaaga | |
| | aggacgcccctagactgcccagaatcctgcccgctggccgtgatgtttccgccttcagcaaactcatgtatgccc | |
| | tgaccatgttcctggatggcaaggagatcaacgacctcctgaccaccctgattaataaattcgataacatccaga | |
| | gcttcctgaaggtgatgcctctcatcggagtcaacgctaagttcgtggaggaatacgcctttttcaaagactccgc | |
| | caagatcgccgatgagctgaggctgatcaagtccttcgctagaatgggagaacctattgccgatgccaggagg | |
| | gccatgtatatcgacgccatccgtattttaggaaccaacctgtcctatgatgagctcaaggccctcgccgacacct | |
| | tttccctggacgagaacggaaacaagctcaagaaaggcaagcacggcatgagaaatttcattattaataacgtg | |
| | atcagcaataaaaggttccactacctgatcagatacggtgatcctgcccacctccatgagatcgccaaaaacga | |
| | ggccgtggtgaagttcgtgctcggcaggatcgctgacatccagaaaaaacagggccagaacggcaagaacc | |
| | agatcgacaggtactacgaaacttgtatcggaaaggataagggcaagagcgtgagcgaaaaggtggacgctc | |
| | tcacaaagatcatcaccggaatgaactacgaccaattcgacaagaaaaggagcgtcattgaggacaccggcag | |
| | ggaaaacgccgagagggagaagtttaaaaagatcatcagcctgtacctcaccgtgatctaccacatcctcaaga | |
| | atattgtcaatatcaacgccaggtacgtcatcggattccattgcgtcgagcgtgatgctcaactgtacaaggagaa | |
| | aggctacgacatcaatctcaagaaactggaagagaagggattcagctccgtcaccaagctctgcgctggcattg | |
| | atgaaactgcccccgataagagaaaggacgtggaaaaggagatggctgaaagagccaaggagagcattgac | |
| | agcctcgagagcgccaaccccaagctgtatgccaattacatcaaatacagcgacgagaagaaagccgaggag | |
| | ttcaccaggcagattaacagggagaaggccaaaaccgccctgaacgcctacctgaggaacaccaagtggaat | |
| | gtgatcatcagggaggacctcctgagaattgacaacaagacatgtaccctgttcagaaacaaggccgtccacct | |
| | ggaagtggccaggtatgtccacgcctatatcaacgacattgccgaggtcaattcctacttccaactgtaccattac | |
| | atcatgcagagaattatcatgaatgagaggtacgagaaaagcagcggaaaggtgtccgagtacttcgacgctgt | |
| | gaatgacgagaagaagtacaacgataggctcctgaaactgctgtgtgtgcctttcggctactgtatccccaggttt | |
| | aagaacctgagcatcgaggccctgttcgataggaacgaggccgccaagttcgacaaggagaaaaagaaggt | |
| | gtccggcaattcc | |
| 7 | MNIPALVENQKKYFGTYSVMAMLNAQTVLDHIQKVADIEGEQNENNE | PspCas13b |
| | NLWFHPVMSHLYNAKNGYDKQPEKTMFIIERLQSYFPFLKIMAENQRE | amino |
| | YSNGKYKQNRVEVNSNDIFEVLKRAFGVLKMYRDLTNHYKTYEEKL | acid |
| | NDGCEFLTSTEQPLSGMINNYYTVALRNMNERYGYKTEDLAFIQDKRF | sequence |
| | KFVKDAYGKKKSQVNTGFFLSLQDYNGDTQKKLHLSGVGIALLICLFL | |
| | DKQYINIFLSRLPIFSSYNAQSEERRIIIRSFGINSIKLPKDRIHSEKSNKSV | |
| | AMDMLNEVKRCPDELFTTLSAEKQSRFRIISDDHNEVLMKRSSDRFVP | |
| | LLLQYIDYGKLFDHIRFHVNMGKLRYLLKADKTCIDGQTRVRVIEQPL | |
| | NGFGRLEEAETMRKQENGTFGNSGIRIRDFENMKRDDANPANYPYIVD | |
| | TYTHYILENNKVEMFINDKEDSAPLLPVIEDDRYVVKTIPSCRMSTLEIP | |
| | AMAFHMFLFGSKKTEKLIVDVHNRYKRLFQAMQKEEVTAENIASFGIA | |
| | ESDLPQKILDLISGNAHGKDVDAFIRLTVDDMLTDTERRIKRFKDDRKS | |
| | IRSADNKMGKRGFKQISTGKLADFLAKDIVLFQPSVNDGENKITGLNY | |
| | RIMQSAIAVYDSGDDYEAKQQFKLMFEKARLIGKGTTEPHPFLYKVFA | |
| | RSIPANAVEFYERYLIERKFYLTGLSNEIKKGNRVDVPFIRRDQNKWKT | |
| | PAMKTLGRIYSEDLPVELPRQMFDNEIKSHLKSLPQMEGIDENNANVT | |
| | YLIAEYMKRVLDDDFQTFYQWNRNYRYMDMLKGEYDRKGSLQHCFT | |
| | SVEEREGLWKERASRTERYRKQASNKIRSNRQMRNASSEEIETILDKRL | |
| | SNSRNEYQKSEKVIRRYRVQDALLFLLAKKTLTELADFDGERFKLKEI | |
| | MPDAEKGILSEIMPMSFTFEKGGKKYTITSEGMKLKNYGDFFVLASDK | |
| | RIGNLLELVGSDIVSKEDIMEEFNKYDQCRPEISSIVFNLEKWAFDTYPE | |
| | LSARVDREEKVDFKSILKILLNNKNINKEQSDILRKIRNAFDHNNYPDK | |
| | GVVEIKALPEIAMSIKKAFGEYAIMK* | |
| 8 | atgaacatccccgctctggtggaaaaccagaagaagtactttggcacctacagcgtgatggccatgctgaacgc | PspCas13b |
| | tcagaccgtgctggaccacatccagaaggtggccgatattgagggcgagcagaacgagaacaacgagaatct | nucleic |
| | gtggtttcaccccgtgatgagccacctgtacaacgccaagaacggctacgacaagcagcccgagaaaaccat | acid |
| | gttcatcatcgagcggctgcagagctacttcccattcctgaagatcatggccgagaaccagagagagtacagca | sequence |
| | acggcaagtacaagcagaaccgcgtggaagtgaacagcaacgacatcttcgaggtgctgaagcgcgccttcg | |
| | gcgtgctgaagatgtacagggacctgaccaaccactacaagacctacgaggaaaagctgaacgacggctgcg | |
| | agttcctgaccagcacagagcaacctctgagcggcatgatcaacaactactacacagtggccctgcggaacat | |
| | gaacgagagatacggctacaagacagaggacctggccttcatccaggacaagcggttcaagttcgtgaagga | |
| | cgcctacggcaagaaaaagtcccaagtgaataccggattcttcctgagcctgcaggactacaacggcgacaca | |
| | cagaagaagctgcacctgagcggagtgggaatcgccctgctgatctgcctgttcctggacaagcagtacatcaa | |
| | catctttctgagcaggctgcccatcttctccagctacaatgcccagagcgaggaacggcggatcatcatcagatc | |
| | cttcggcatcaacagcatcaagctgcccaaggaccggatccacagcgagaagtccaacaagagcgtggccat | |
| | ggatatgctcaacgaagtgaagcggtgccccgacgagctgttcacaacactgtctgccgagaagcagtcccgg | |
| | ttcagaatcatcagcgacgaccacaatgaagtgctgatgaagcggagcagcgacagattcgtgcctctgctgct | |
| | gcagtatatcgattacggcaagctgttcgaccacatcaggttccacgtgaacatgggcaagctgagatacctgct | |
| | gaaggccgacaagacctgcatcgacggccagaccagagtcagagtgatcgagcagcccctgaacggcttcg | |
| | gcagactggaagaggccgagacaatgcggaagcaagagaacggcaccttcggcaacagcggcatccggat | |
| | cagagacttcgagaacatgaagcgggacgacgccaatcctgccaactatccctacatcgtggacacctacaca | |
| | cactacatcctggaaaacaacaaggtcgagatgtttatcaacgacaaagaggacagcgccccactgctgcccg | |
| | tgatcgaggatgatagatacgtggtcaagacaatccccagctgccggatgagcaccctggaaattccagccatg | |
| | gccttccacatgtttctgttcggcagcaagaaaaccgagaagctgatcgtggacgtgcacaaccggtacaagag | |
| | actgttccaggccatgcagaaagaagaagtgaccgccgagaatatcgccagcttcggaatcgccgagagcga | |
| | cctgcctcagaagatcctggatctgatcagcggcaatgcccacggcaaggatgtggacgccttcatcagactga | |
| | ccgtggacgacatgctgaccgacaccgagcggagaatcaagagattcaaggacgaccggaagtccattcgga | |
| | gcgccgacaacaagatgggaaagagaggcttcaagcagatctccacaggcaagctggccgacttcctggcca | |
| | aggacatcgtgctgtttcagcccagcgtgaacgatggcgagaacaagatcaccggcctgaactaccggatcat | |
| | gcagagcgccattgccgtgtacgatagcggcgacgattacgaggccaagcagcagttcaagctgatgttcgag | |
| | aaggcccggctgatcggcaagggcacaacagagcctcatccatttctgtacaaggtgttcgcccgcagcatcc | |
| | ccgccaatgccgtcgagttctacgagcgctacctgatcgagcggaagttctacctgaccggcctgtccaacgag | |
| | atcaagaaaggcaacagagtggatgtgcccttcatccggcgggaccagaacaagtggaaaacacccgccatg | |
| | aagaccctgggcagaatctacagcgaggatctgcccgtggaactgcccagacagatgttcgacaatgagatca | |
| | agtcccacctgaagtccctgccacagatggaaggcatcgacttcaacaatgccaacgtgacctatctgatcgcc | |
| | gagtacatgaagagagtgctggacgacgacttccagaccttctaccagtggaaccgcaactaccggtacatgg | |
| | acatgcttaagggcgagtacgacagaaagggctccctgcagcactgcttcaccagcgtggaagagagagaag | |
| | gcctctggaaagagcgggcctccagaacagagcggtacagaaagcaggccagcaacaagatccgcagcaa | |
| | ccggcagatgagaaacgccagcagcgaagagatcgagacaatcctggataagcggctgagcaacagccgg | |
| | aacgagtaccagaaaagcgagaaagtgatccggcgctacagagtgcaggatgccctgctgtttctgctggcca | |
| | aaaagaccctgaccgaactggccgatttcgacggcgagaggttcaaactgaaagaaatcatgcccgacgccg | |
| | agaagggaatcctgagcgagatcatgcccatgagcttcaccttcgagaaaggcggcaagaagtacaccatcac | |
| | cagcgagggcatgaagctgaagaactacggcgacttctttgtgctggctagcgacaagaggatcggcaacctg | |
| | ctggaactcgtgggcagcgacatcgtgtccaaagaggatatcatggaagagttcaacaaatacgaccagtgca | |
| | ggcccgagatcagctccatcgtgttcaacctggaaaagtgggccttcgacacataccccgagctgtctgccaga | |
| | gtggaccgggaagagaaggtggacttcaagagcatcctgaaaatcctgctgaacaacaagaacatcaacaaa | |
| | gagcagagcgacatcctgcggaagatccggaacgccttcgatcacaacaattaccccgacaaaggcgtggtg | |
| | gaaatcaaggccctgcctgagatcgccatgagcatcaagaaggcctttggggagtacgccatcatgaag | |

What is claimed is:

1. A method of screening for a nuclease with an altered nuclease activity comprising:
a) forming a first compartmentalizing reaction comprising a carrier and a variant nuclease template nucleic acid comprising a coding region for a variant nuclease, and amplifying said coding region for said variant nuclease, to obtain variant nuclease e encoding nucleic acid, wherein said variant nuclease encoding nucleic acid is complexed to said carrier after said amplifying;
b) forming a second compartmentalizing reaction comprising said variant nuclease encoding nucleic acid, and performing an in vitro transcription and translation reaction to obtain variant nuclease polypeptides; and
c) assaying said variant nuclease polypeptides for the altered nuclease activity of said nuclease.

2. The method of claim 1, wherein said nuclease is a DNA nuclease or RNA nuclease.

3. The method of claim 2, wherein said nuclease is a Cas nuclease.

4. The method of claim 3, wherein said nuclease is a Cas13 nuclease.

5. The method of claim 3, wherein said nuclease is a Cas12 nuclease.

6. The method of claim 3, wherein said Cas nuclease is a nuclease selected from the list consisting of a Cas12a, a Cas12b, a Cas12d, a Cas13a, a Cas13b, a Cas13d, and a CasRx.

7. The method of claim 3, wherein said nuclease is a Cas nuclease having collateral nuclease activity.

8. The method of claim 6, wherein said nuclease is a selected from the list consisting of Leptotrichia wadei Cas13 (LwaCas13a) nuclease, Leptotrichia buccalis Cas13 (LbuCas13a) nuclease, Ruminococcus flavefaciens XPD3002 (RfxCas13d) and Prevotella sp. P5-125 (PspCas13b).

9. The method of claim 8, wherein said nuclease comprises an amino acid sequence at least 85% identical to the amino acid sequence set forth in SEQ ID NO: 1, SEQ ID NO: 3, SEQ ID NO: 5, or SEQ ID NO: 7.

10. The method of claim 8, wherein said nuclease comprises an amino acid sequence at least 90% identical to the amino acid sequence set forth in SEQ ID NO: 1, SEQ ID NO: 3, SEQ ID NO: 5, or SEQ ID NO: 7.

11. The method of claim 8, wherein said nuclease comprises an amino acid sequence at least 95% identical to the amino acid sequence set forth in SEQ ID NO: 1, SEQ ID NO: 3, SEQ ID NO: 5, or SEQ ID NO: 7.

12. The method of claim 8, wherein said nuclease comprises the amino acid sequence set forth in SEQ ID NO: 1, SEQ ID NO: 3, SEQ ID NO: 5, or SEQ ID NO: 7.

13. The method of claim 8, wherein said nuclease is encoded by a nucleic acid possessing at least 85% homology to SEQ ID NO: 2, SEQ ID NO: 4, SEQ ID NO: 6, or SEQ ID NO: 8.

14. The method of claim 8, wherein said nuclease is encoded by a nucleic acid possessing at least 90% homology to SEQ ID NO: 2, SEQ ID NO: 4, SEQ ID NO: 6, or SEQ ID NO: 8.

15. The method of claim 8, wherein said nuclease is encoded by a nucleic acid possessing at least 95% homology to SEQ ID NO: 2, SEQ ID NO: 4, SEQ ID NO: 6, or SEQ ID NO: 8.

16. The method of claim 8, wherein said variant nuclease comprises an amino sequencing possessing one or more amino acids substitutions, deletions, or insertions compared to SEQ ID NO: 1, SEQ ID NO: 3, SEQ ID NO: 5, or SEQ ID NO: 7.

17. The method of claim 3, wherein said Cas nuclease is a variant Cas13 nuclease of a type VI-A CRISPR-Cas system and is characterized by cleavage activity of a single-stranded RNA.

18. The method of claim 1, wherein said nuclease is a bacterial nuclease selected from the list consisting of Leptotrichia wadei, Leptotrichia buccalis, Leptotrichia shahii, Leptotrichia massiliensis, Leptotrichia trevisanii, Herbinix hemicellulosilytica, and Escherichia coli, Ruminococcus flavefaciens, Prevotella sp. P5-125, and Porphyromona gulae.

19. The method of claim 1, wherein said first compartmentalizing reaction comprises one or more of dNTPs, a polymerase, and primers complementary to said coding region for a variant nuclease.

20. The method of claim 1, wherein said first compartmentalizing reaction further comprises a guide nucleic acid, wherein said guide RNA comprises one or more of; a non-natural internucleoside linkage, a nucleic acid mimetic, a modified sugar moiety, and a modified nucleobase.

21. The method of claim 1, wherein said carrier is a bead selected from the list consisting of magnetic material, glass, polyacrylamide, polystyrene, protein A, protein G, streptavidin, antibodies, and silanized material.

22. The method of claim 1, wherein said variant nuclease polypeptide binds to said guide nucleic acid in said second compartmentalizing reaction.

23. The method of claim 1, wherein assaying said variant nuclease polypeptides for the altered nuclease activity of said nuclease is performed in a third compartmentalizing reaction, wherein said third compartmentalizing reaction comprises said variant nuclease polypeptides and a fluorescence reporter system.

24. The method of claim 1, wherein said altered nuclease activity is an increase or decrease in nuclease activity, wherein said increase in nuclease activity or said decrease in nuclease activity is trans nuclease activity or is cis nuclease activity.

25. The method of claim 1, wherein any one or more of said first compartmentalizing reaction, second compartmentalizing reaction, and third compartmentalizing reaction is an oil and water emulsion.

26. The method of claim 1, wherein any one or more of said first compartmentalizing reaction, second compartmentalizing reaction, and third compartmentalizing reaction is an oil and water double emulsion.

27. A variant nuclease produced by the method of claim 1.


SEQUENCE LISTING (22 sequences)

SEQ ID NO: 1 - protein, 1152 aa, Leptotrichia wadei
MKVTKVDGISHKKYIEEGKLVKSTSEENRTSERLSELLSIRLDIYIKNPDNASEEENRIRRENLKKFFSNKVLHLKDSVLYLKNRKEKNAVQDKNYSEEDISEYDLKNKNSFSVLKKILLNEDVNSEELEIFRKDVEAKLNKINSLKYSFEENKANYQKINENNVEKVGGKSKRNIIYDYYRESAKRNDYINNVQEAFDKLYKKEDIEKLFFLIENSKKHEKYKIREYYHKIIGRKNDKENFAKIIYEEIQNVNNIKELIEKIPDMSELKKSQVFYKYYLDKEELNDKNIKYAFCHFVEIEMSQLLKNYVYKRLSNISNDKIKRIFEYQNLKKLIENKLLNKLDTYVRNCGKYNYYLQVGEIATSDFIARNRQNEAFLRNIIGVSSVAYFSLRNILETENENDITGRMRGKTVKNNKGEEKYVSGEVDKIYNENKQNEVKENLKMFYSYDFNMDNKNEIEDFFANIDEAISSIRHGIVHFNLELEGKDIFAFKNIAPSEISKKMFQNEINEKKLKLKIFKQLNSANVFNYYEKDVIIKYLKNTKFNFVNKNIPFVPSFTKLYNKIEDLRNTLKFFWSVPKDKEEKDAQIYLLKNIYYGEFLNKFVKNSKVFFKITNEVIKINKQRNQKTGHYKYQKFENIEKTVPVEYLAIIQSREMINNQDKEEKNTYIDFIQQIFLKGFIDYLNKNNLKYIESNNNNDNNDIFSKIKIKKDNKEKYDKILKNYEKHNRNKEIPHEINEFVREIKLGKILKYTENLNMFYLILKLLNHKELTNLKGSLEKYQSANKEETFSDELELINLLNLDNNRVTEDFELEANEIGKFLDFNENKIKDRKELKKFDTNKIYFDGENIIKHRAFYNIKKYGMLNLLEKIADKAKYKISLKELKEYSNKKNEIEKNYTMQQNLHRKYARPKKDEKFNDEDYKEYEKAIGNIQKYTHLKNKVEFNELNLLQGLLLKILHRLVGYTSIWERDLRFRLKGEFPENHYIEEIFNFDNSKNVKYKSGQIVEKYINFYKELYKDNVEKRSIYSDKKVKKLKQEKKDLYIRNYIAHFNYIPHAEISLLEVLENLRKLLSYDRKLKNAIMKSIVDILKEYGFVATFKIGADKKIEIQTLESEKIVHLKNLKKKKLMTDRNSEELCELVKVMFEYKALE

SEQ ID NO: 2 - DNA, 3459 bp, Leptotrichia wadei
  source 1..3459: mol_type = genomic DNA
atgaaagtgaccaaggtcgacggcatcagccacaagaagtacatcgaagagggcaagctcgtgaagtccaccagcgaggaaaaccggaccagcgagagactgagcgagctgctgagcatccggctggacatctacatcaagaaccccgacaacgcctccgaggaagagaaccggatcagaagagagaacctgaagaagttctttagcaacaaggtgctgcacctgaaggacagcgtgctgtatctgaagaaccggaaagaaaagaacgccgtgcaggacaagaactatagcgaagaggacatcagcgagtacgacctgaaaaacaagaacagcttctccgtgctgaagaagatcctgctgaacgaggacgtgaactctgaggaactggaaatctttcggaaggacgtggaagccaagctgaacaagatcaacagcctgaagtacagcttcgaagagaacaaggccaactaccagaagatcaacgagaacaacgtggaaaaagtgggcggcaagagcaagcggaacatcatctacgactactacagagagagcgccaagcgcaacgactacatcaacaacgtgcaggaagccttcgacaagctgtataagaaagaggatatcgagaaactgtttttcctgatcgagaacagcaagaagcacgagaagtacaagatccgcgagtactatcacaagatcatcggccggaagaacgacaaagagaacttcgccaagattatctacgaagagatccagaacgtgaacaacatcaaagagctgattgagaagatccccgacatgtctgagctgaagaaaagccaggtgttctacaagtactacctggacaaagaggaactgaacgacaagaatattaagtacgccttctgccacttcgtggaaatcgagatgtcccagctgctgaaaaactacgtgtacaagcggctgagcaacatcagcaacgataagatcaagcggatcttcgagtaccagaatctgaaaaagctgatcgaaaacaaactgctgaacaagctggacacctacgtgcggaactgcggcaagtacaactactatctgcaagtgggcgagatcgccacctccgactttatcgcccggaaccggcagaacgaggccttcctgagaaacatcatcggcgtgtccagcgtggcctacttcagcctgaggaacatcctggaaaccgagaacgagaacgatatcaccggccggatgcggggcaagaccgtgaagaacaacaagggcgaagagaaatacgtgtccggcgaggtggacaagatctacaatgagaacaagcagaacgaagtgaaagaaaatctgaagatgttctacagctacgacttcaacatggacaacaagaacgagatcgaggacttcttcgccaacatcgacgaggccatcagcagcatcagacacggcatcgtgcacttcaacctggaactggaaggcaaggacatcttcgccttcaagaatatcgcccccagcgagatctccaagaagatgtttcagaacgaaatcaacgaaaagaagctgaagctgaaaatcttcaagcagctgaacagcgccaacgtgttcaactactacgagaaggatgtgatcatcaagtacctgaagaataccaagttcaacttcgtgaacaaaaacatccccttcgtgcccagcttcaccaagctgtacaacaagattgaggacctgcggaataccctgaagtttttttggagcgtgcccaaggacaaagaagagaaggacgcccagatctacctgctgaagaatatctactacggcgagttcctgaacaagttcgtgaaaaactccaaggtgttctttaagatcaccaatgaagtgatcaagattaacaagcagcggaaccagaaaaccggccactacaagtatcagaagttcgagaacatcgagaaaaccgtgcccgtggaatacctggccatcatccagagcagagagatgatcaacaaccaggacaaagaggaaaagaatacctacatcgactttattcagcagattttcctgaagggcttcatcgactacctgaacaagaacaatctgaagtatatcgagagcaacaacaacaatgacaacaacgacatcttctccaagatcaagatcaaaaaggataacaaagagaagtacgacaagatcctgaagaactatgagaagcacaatcggaacaaagaaatccctcacgagatcaatgagttcgtgcgcgagatcaagctggggaagattctgaagtacaccgagaatctgaacatgttttacctgatcctgaagctgctgaaccacaaagagctgaccaacctgaagggcagcctggaaaagtaccagtccgccaacaaagaagaaaccttcagcgacgagctggaactgatcaacctgctgaacctggacaacaacagagtgaccgaggacttcgagctggaagccaacgagatcggcaagttcctggacttcaacgaaaacaaaatcaaggaccggaaagagctgaaaaagttcgacaccaacaagatctatttcgacggcgagaacatcatcaagcaccgggccttctacaatatcaagaaatacggcatgctgaatctgctggaaaagatcgccgataaggccaagtataagatcagcctgaaagaactgaaagagtacagcaacaagaagaatgagattgaaaagaactacaccatgcagcagaacctgcaccggaagtacgccagacccaagaaggacgaaaagttcaacgacgaggactacaaagagtatgagaaggccatcggcaacatccagaagtacacccacctgaagaacaaggtggaattcaatgagctgaacctgctgcagggcctgctgctgaagatcctgcaccggctcgtgggctacaccagcatctgggagcgggacctgagattccggctgaagggcgagtttcccgagaaccactacatcgaggaaattttcaatttcgacaactccaagaatgtgaagtacaaaagcggccagatcgtggaaaagtatatcaacttctacaaagaactgtacaaggacaatgtggaaaagcggagcatctactccgacaagaaagtgaagaaactgaagcaggaaaaaaaggacctgtacatccggaactacattgcccacttcaactacatcccccacgccgagattagcctgctggaagtgctggaaaacctgcggaagctgctgtcctacgaccggaagctgaagaacgccatcatgaagtccatcgtggacattctgaaagaatacggcttcgtggccaccttcaagatcggcgctgacaagaagatcgaaatccagaccctggaatcagagaagatcgtgcacctgaagaatctgaagaaaaagaaactgatgaccgaccggaacagcgaggaactgtgcgaactcgtgaaagtcatgttcgagtacaaggccctggaataa

SEQ ID NO: 3 - protein, 1159 aa, Leptotrichia buccalis
MKVTKVGGISHKKYTSEGRLVKSESEENRTDERLSALLNMRLDMYIKNPSSTETKENQKRIGKLKKFFSNKMVYLKDNTLSLKNGKKENIDREYSETDILESDVRDKKNFAVLKKIYLNENVNSEELEVFRNDIKKKLNKINSLKYSFEKNKANYQKINENNIEKVEGKSKRNIIYDYYRESAKRDAYVSNVKEAFDKLYKEEDIAKLVLEIENLTKLEKYKIREFYHEIIGRKNDKENFAKIIYEEIQNVNNMKELIEKVPDMSELKKSQVFYKYYLDKEELNDKNIKYAFCHFVEIEMSQLLKNYVYKRLSNISNDKIKRIFEYQNLKKLIENKLLNKLDTYVRNCGKYNYYLQDGEIATSDFIARNRQNEAFLRNIIGVSSVAYFSLRNILETENENDITGRMRGKTVKNNKGEEKYVSGEVDKIYNENKKNEVKENLKMFYSYDFNMDNKNEIEDFFANIDEAISSIRHGIVHFNLELEGKDIFAFKNIAPSEISKKMFQNEINEKKLKLKIFRQLNSANVFRYLEKYKILNYLKRTRFEFVNKNIPFVPSFTKLYSRIDDLKNSLGIYWKTPKTNDDNKTKEIIDAQIYLLKNIYYGEFLNYFMSNNGNFFEISKEIIELNKNDKRNLKTGFYKLQKFEDIQEKIPKEYLANIQSLYMINAGNQDEEEKDTYIDFIQKIFLKGFMTYLANNGRLSLIYIGSDEETNTSLAEKKQEFDKFLKKYEQNNNIKIPYEINEFLREIKLGNILKYTERLNMFYLILKLLNHKELTNLKGSLEKYQSANKEEAFSDQLELINLLNLDNNRVTEDFELEADEIGKFLDFNGNKVKDNKELKKFDTNKIYFDGENIIKHRAFYNIKKYGMLNLLEKIADKAGYKISIEELKKYSNKKNEIEKNHKMQENLHRKYARPRKDEKFTDEDYESYKQAIENIEEYTHLKNKVEFNELNLLQGLLLRILHRLVGYTSIWERDLRFRLKGEFPENQYIEEIFNFENKKNVKYKGGQIVEKYIKFYKELHQNDEVKINKYSSANIKVLKQEKKDLYIRNYIAHFNYIPHAEISLLEVLENLRKLLSYDRKLKNAVMKSVVDILKEYGFVATFKIGADKKIGIQTLESEKIVHLKNLKKKKLMTDRNSEELCKLVKIMFEYKMEEKKSEN

SEQ ID NO: 4 - DNA, 3480 bp, Leptotrichia buccalis
  source 1..3480: mol_type = genomic DNA
atgaaagttacaaaagttggaggaatttcacataaaaaatatactagtgaaggaaggcttgtaaaaagcgaaagtgaagaaaataggactgatgagagattatctgcacttttaaatatgagactagatatgtatataaaaaatcctagcagtacagaaacaaaagaaaatcaaaaaagaataggaaaactgaaaaaattcttttcaaataaaatggtgtatttaaaagacaataccttaagcttaaagaatggaaaaaaggaaaatattgacagggaatattctgaaacagatattttggaatccgatgtaagagataaaaaaaattttgcggttttaaaaaaaatatatttaaatgaaaatgtaaattcagaagaattggaagtatttagaaacgatattaagaaaaaattgaataaaataaattctttaaaatattcatttgaaaaaaataaggctaattaccagaaaattaatgaaaataatattgaaaaagttgaaggaaagagcaaaagaaatattatttatgattattacagagaatcagcaaaacgtgatgcttatgtgagtaatgtaaaagaagcctttgataaattatataaagaagaggatattgccaagttagttttagaaatagaaaatttaacaaagcttgaaaaatataaaataagagaattttatcacgaaataattggaagaaaaaatgataaagaaaattttgctaaaattatttatgaagaaatacaaaatgtaaataatatgaaagaattaattgaaaaagttccagatatgagtgaattaaaaaaatcacaagtgttttataaatattatttggataaagaagaacttaatgataaaaatataaaatatgctttttgtcattttgtggaaattgaaatgagtcagcttttgaaaaattatgtgtataaaagactgagtaatataagtaacgataaaattaagagaatatttgaatatcaaaatttaaaaaaattaattgaaaataaattactaaataaattggatacttatgtaagaaattgcgggaaatataattattatttacaagatggagaaattgcaacaagtgattttattgctaggaatagacaaaatgaagcatttttacgaaatataattggagtttcttcggttgcatatttttcattgagaaatattcttgaaactgaaaatgagaatgatattacaggtagaatgagaggaaagactgtaaaaaataataagggcgaagaaaaatatgtttctggagaagttgataaaatatacaatgaaaacaagaaaaatgaagtaaaagaaaatttaaaaatgttctatagttatgattttaatatggataataaaaatgagatagaagattttttcgcaaatattgatgaagctattagcagtattagacatgggattgtgcattttaatttggaattagaagggaaagatatatttgcatttaaaaatatagctccttctgaaatttcaaaaaaaatgtttcaaaatgaaataaatgagaaaaaattgaaattgaagatatttaggcagttgaatagtgcgaatgtatttaggtatttggaaaaatataaaatattaaattatttaaagagaacacgatttgagtttgttaataaaaatattccatttgttccgtcatttacaaaattgtacagcaggatagatgatttaaaaaatagtctaggtatttattggaaaactccgaaaacaaatgatgataataaaactaaagaaattatagatgctcagatatatcttttgaaaaatatttattatggagaatttttaaattattttatgagcaataacgggaatttttttgagataagcaaagaaataattgaattaaataaaaatgataaaagaaatttaaaaactggattttataaactacaaaaatttgaagatattcaagaaaaaattcctaaagaatatcttgcaaatatacaaagcctttatatgattaatgctggaaatcaggatgaagaggaaaaagatacatatattgactttatacaaaaaatatttttaaaaggatttatgacttacttggcaaataacggaagattaagcctaatatatattggaagcgatgaagaaacaaatacttctttagcagaaaaaaagcaagaatttgataaattcttgaaaaaatatgaacaaaataataatattaaaattccatatgaaataaatgaatttttaagagagataaaattaggaaatatattaaaatacaccgaaagattgaacatgttttatttaattttaaaattgcttaatcataaagaattgactaatttgaaaggaagtcttgaaaaatatcagagtgcaaataaagaagaagctttttcagatcaacttgaacttataaatcttttaaatttagataataatagagtaacagaagattttgaattagaagcggatgaaattgggaagtttttagattttaatggaaataaggtaaaagataataaagaattgaagaaatttgacacaaataaaatatattttgatggagaaaatattataaagcatagagctttttataatataaaaaaatatgggatgttaaatttacttgagaaaatagcggataaggcaggatataaaataagtatagaagaattgaaaaaatacagtaataaaaaaaatgaaattgaaaaaaatcataaaatgcaggaaaatttacatagaaaatatgcaagacctagaaaagatgaaaaatttacagatgaagattatgaaagctataaacaggcaattgaaaatatagaagaatatactcatttgaaaaataaagtagaatttaatgagttaaatttgttacaaggtctactattaagaatacttcatagacttgtggggtatacttcgatttgggaaagagatttgagatttagattaaaaggggaatttccagaaaatcagtatatagaagaaatatttaattttgagaataaaaagaatgtgaaatataaaggtggacaaattgttgaaaagtatataaaattttataaggaattacatcaaaatgatgaagtaaaaataaataaatattccagtgcaaatataaaagtgttaaaacaagagaaaaaagatttgtatatacgaaactatattgcacatttcaactatattccgcatgctgaaatttcacttttagaagtgctggaaaatttaagaaaactactttcttatgatagaaaacttaaaaatgcagttatgaaatcagtagtggatatattaaaggaatatggttttgtagcaacatttaaaataggagcagataagaaaataggaattcagactttagagtcggaaaaaattgtacatttgaaaaatttaaaaaagaaaaaattaatgactgatagaaattcagaagaattgtgtaaacttgtgaagattatgtttgaatataaaatggaagaaaaaaaatctgaaaactaa

SEQ ID NO: 5 - protein, 967 aa, Ruminococcus flavefaciens
MIEKKKSFAKGMGVKSTLVSGSKVYMTTFAEGSDARLEKIVEGDSIRSVNEGEAFSAEMADKNAGYKIGNAKFSHPKGYAVVANNPLYTGPVQQDMLGLKETLEKRYFGESADGNDNICIQVIHNILDIEKILAEYITNAAYAVNNISGLDKDIIGFGKFSTVYTYDEFKDPEHHRAAFNNNDKLINAIKAQYDEFDNFLDNPRLGYFGQAFFSKEGRNYIINYGNECYDILALLSGLRHWVVHNNEEESRISRTWLYNLDKNLDNEYISTLNYLYDRITNELTNSFSKNSAANVNYIAETLGINPAEFAEQYFRFSIMKEQKNLGFNITKLREVMLDRKDMSEIRKNHKVFDSIRTKVYTMMDFVIYRYYIEEDAKVAAANKSLPDNEKSLSEKDIFVINLRGSFNDDQKDALYYDEANRIWRKLENIMHNIKEFRGNKTREYKKKDAPRLPRILPAGRDVSAFSKLMYALTMFLDGKEINDLLTTLINKFDNIQSFLKVMPLIGVNAKFVEEYAFFKDSAKIADELRLIKSFARMGEPIADARRAMYIDAIRILGTNLSYDELKALADTFSLDENGNKLKKGKHGMRNFIINNVISNKRFHYLIRYGDPAHLHEIAKNEAVVKFVLGRIADIQKKQGQNGKNQIDRYYETCIGKDKGKSVSEKVDALTKIITGMNYDQFDKKRSVIEDTGRENAEREKFKKIISLYLTVIYHILKNIVNINARYVIGFHCVERDAQLYKEKGYDINLKKLEEKGFSSVTKLCAGIDETAPDKRKDVEKEMAERAKESIDSLESANPKLYANYIKYSDEKKAEEFTRQINREKAKTALNAYLRNTKWNVIIREDLLRIDNKTCTLFRNKAVHLEVARYVHAYINDIAEVNSYFQLYHYIMQRIIMNERYEKSSGKVSEYFDAVNDEKKYNDRLLKLLCVPFGYCIPRFKNLSIEALFDRNEAAKFDKEKKKVSGNS

SEQ ID NO: 6 - DNA, 2898 bp, Ruminococcus flavefaciens
  source 1..2898: mol_type = genomic DNA
atcgaaaaaaaaaagtccttcgccaagggcatgggcgtgaagtccacactcgtgtccggctccaaagtgtacatgacaaccttcgccgaaggcagcgacgccaggctggaaaagatcgtggagggcgacagcatcaggagcgtgaatgagggcgaggccttcagcgctgaaatggccgataaaaacgccggctataagatcggcaacgccaaattcagccatcctaagggctacgccgtggtggctaacaaccctctgtatacaggacccgtccagcaggatatgctcggcctgaaggaaactctggaaaagaggtacttcggcgagagcgctgatggcaatgacaatatttgtatccaggtgatccataacatcctggacattgaaaaaatcctcgccgaatacattaccaacgccgcctacgccgtcaacaatatctccggcctggataaggacattattggattcggcaagttctccacagtgtatacctacgacgaattcaaagaccccgagcaccatagggccgctttcaacaataacgataagctcatcaacgccatcaaggcccagtatgacgagttcgacaacttcctcgataaccccagactcggctatttcggccaggcctttttcagcaaggagggcagaaattacatcatcaattacggcaacgaatgctatgacattctggccctcctgagcggactgaggcactgggtggtccataacaacgaagaagagtccaggatctccaggacctggctctacaacctcgataagaacctcgacaacgaatacatctccaccctcaactacctctacgacaggatcaccaatgagctgaccaactccttctccaagaactccgccgccaacgtgaactatattgccgaaactctgggaatcaaccctgccgaattcgccgaacaatatttcagattcagcattatgaaagagcagaaaaacctcggattcaatatcaccaagctcagggaagtgatgctggacaggaaggatatgtccgagatcaggaaaaatcataaggtgttcgactccatcaggaccaaggtctacaccatgatggactttgtgatttataggtattacatcgaagaggatgccaaggtggctgccgccaataagtccctccccgataatgagaagtccctgagcgagaaggatatctttgtgattaacctgaggggctccttcaacgacgaccagaaggatgccctctactacgatgaagctaatagaatttggagaaagctcgaaaatatcatgcacaacatcaaggaatttaggggaaacaagacaagagagtataagaagaaggacgcccctagactgcccagaatcctgcccgctggccgtgatgtttccgccttcagcaaactcatgtatgccctgaccatgttcctggatggcaaggagatcaacgacctcctgaccaccctgattaataaattcgataacatccagagcttcctgaaggtgatgcctctcatcggagtcaacgctaagttcgtggaggaatacgcctttttcaaagactccgccaagatcgccgatgagctgaggctgatcaagtccttcgctagaatgggagaacctattgccgatgccaggagggccatgtatatcgacgccatccgtattttaggaaccaacctgtcctatgatgagctcaaggccctcgccgacaccttttccctggacgagaacggaaacaagctcaagaaaggcaagcacggcatgagaaatttcattattaataacgtgatcagcaataaaaggttccactacctgatcagatacggtgatcctgcccacctccatgagatcgccaaaaacgaggccgtggtgaagttcgtgctcggcaggatcgctgacatccagaaaaaacagggccagaacggcaagaaccagatcgacaggtactacgaaacttgtatcggaaaggataagggcaagagcgtgagcgaaaaggtggacgctctcacaaagatcatcaccggaatgaactacgaccaattcgacaagaaaaggagcgtcattgaggacaccggcagggaaaacgccgagagggagaagtttaaaaagatcatcagcctgtacctcaccgtgatctaccacatcctcaagaatattgtcaatatcaacgccaggtacgtcatcggattccattgcgtcgagcgtgatgctcaactgtacaaggagaaaggctacgacatcaatctcaagaaactggaagagaagggattcagctccgtcaccaagctctgcgctggcattgatgaaactgcccccgataagagaaaggacgtggaaaaggagatggctgaaagagccaaggagagcattgacagcctcgagagcgccaaccccaagctgtatgccaattacatcaaatacagcgacgagaagaaagccgaggagttcaccaggcagattaacagggagaaggccaaaaccgccctgaacgcctacctgaggaacaccaagtggaatgtgatcatcagggaggacctcctgagaattgacaacaagacatgtaccctgttcagaaacaaggccgtccacctggaagtggccaggtatgtccacgcctatatcaacgacattgccgaggtcaattcctacttccaactgtaccattacatcatgcagagaattatcatgaatgagaggtacgagaaaagcagcggaaaggtgtccgagtacttcgacgctgtgaatgacgagaagaagtacaacgataggctcctgaaactgctgtgtgtgcctttcggctactgtatccccaggtttaagaacctgagcatcgaggccctgttcgataggaacgaggccgccaagttcgacaaggagaaaaagaaggtgtccggcaattcc

SEQ ID NO: 7 - protein, 1090 aa, Prevotella sp.
MNIPALVENQKKYFGTYSVMAMLNAQTVLDHIQKVADIEGEQNENNENLWFHPVMSHLYNAKNGYDKQPEKTMFIIERLQSYFPFLKIMAENQREYSNGKYKQNRVEVNSNDIFEVLKRAFGVLKMYRDLTNHYKTYEEKLNDGCEFLTSTEQPLSGMINNYYTVALRNMNERYGYKTEDLAFIQDKRFKFVKDAYGKKKSQVNTGFFLSLQDYNGDTQKKLHLSGVGIALLICLFLDKQYINIFLSRLPIFSSYNAQSEERRIIIRSFGINSIKLPKDRIHSEKSNKSVAMDMLNEVKRCPDELFTTLSAEKQSRFRIISDDHNEVLMKRSSDRFVPLLLQYIDYGKLFDHIRFHVNMGKLRYLLKADKTCIDGQTRVRVIEQPLNGFGRLEEAETMRKQENGTFGNSGIRIRDFENMKRDDANPANYPYIVDTYTHYILENNKVEMFINDKEDSAPLLPVIEDDRYVVKTIPSCRMSTLEIPAMAFHMFLFGSKKTEKLIVDVHNRYKRLFQAMQKEEVTAENIASFGIAESDLPQKILDLISGNAHGKDVDAFIRLTVDDMLTDTERRIKRFKDDRKSIRSADNKMGKRGFKQISTGKLADFLAKDIVLFQPSVNDGENKITGLNYRIMQSAIAVYDSGDDYEAKQQFKLMFEKARLIGKGTTEPHPFLYKVFARSIPANAVEFYERYLIERKFYLTGLSNEIKKGNRVDVPFIRRDQNKWKTPAMKTLGRIYSEDLPVELPRQMFDNEIKSHLKSLPQMEGIDFNNANVTYLIAEYMKRVLDDDFQTFYQWNRNYRYMDMLKGEYDRKGSLQHCFTSVEEREGLWKERASRTERYRKQASNKIRSNRQMRNASSEEIETILDKRLSNSRNEYQKSEKVIRRYRVQDALLFLLAKKTLTELADFDGERFKLKEIMPDAEKGILSEIMPMSFTFEKGGKKYTITSEGMKLKNYGDFFVLASDKRIGNLLELVGSDIVSKEDIMEEFNKYDQCRPEISSIVFNLEKWAFDTYPELSARVDREEKVDFKSILKILLNNKNINKEQSDILRKIRNAFDHNNYPDKGVVEIKALPEIAMSIKKAFGEYAIMK

SEQ ID NO: 8 - DNA, 3270 bp, Prevotella sp.
  source 1..3270: mol_type = genomic DNA
atgaacatccccgctctggtggaaaaccagaagaagtactttggcacctacagcgtgatggccatgctgaacgctcagaccgtgctggaccacatccagaaggtggccgatattgagggcgagcagaacgagaacaacgagaatctgtggtttcaccccgtgatgagccacctgtacaacgccaagaacggctacgacaagcagcccgagaaaaccatgttcatcatcgagcggctgcagagctacttcccattcctgaagatcatggccgagaaccagagagagtacagcaacggcaagtacaagcagaaccgcgtggaagtgaacagcaacgacatcttcgaggtgctgaagcgcgccttcggcgtgctgaagatgtacagggacctgaccaaccactacaagacctacgaggaaaagctgaacgacggctgcgagttcctgaccagcacagagcaacctctgagcggcatgatcaacaactactacacagtggccctgcggaacatgaacgagagatacggctacaagacagaggacctggccttcatccaggacaagcggttcaagttcgtgaaggacgcctacggcaagaaaaagtcccaagtgaataccggattcttcctgagcctgcaggactacaacggcgacacacagaagaagctgcacctgagcggagtgggaatcgccctgctgatctgcctgttcctggacaagcagtacatcaacatctttctgagcaggctgcccatcttctccagctacaatgcccagagcgaggaacggcggatcatcatcagatccttcggcatcaacagcatcaagctgcccaaggaccggatccacagcgagaagtccaacaagagcgtggccatggatatgctcaacgaagtgaagcggtgccccgacgagctgttcacaacactgtctgccgagaagcagtcccggttcagaatcatcagcgacgaccacaatgaagtgctgatgaagcggagcagcgacagattcgtgcctctgctgctgcagtatatcgattacggcaagctgttcgaccacatcaggttccacgtgaacatgggcaagctgagatacctgctgaaggccgacaagacctgcatcgacggccagaccagagtcagagtgatcgagcagcccctgaacggcttcggcagactggaagaggccgagacaatgcggaagcaagagaacggcaccttcggcaacagcggcatccggatcagagacttcgagaacatgaagcgggacgacgccaatcctgccaactatccctacatcgtggacacctacacacactacatcctggaaaacaacaaggtcgagatgtttatcaacgacaaagaggacagcgccccactgctgcccgtgatcgaggatgatagatacgtggtcaagacaatccccagctgccggatgagcaccctggaaattccagccatggccttccacatgtttctgttcggcagcaagaaaaccgagaagctgatcgtggacgtgcacaaccggtacaagagactgttccaggccatgcagaaagaagaagtgaccgccgagaatatcgccagcttcggaatcgccgagagcgacctgcctcagaagatcctggatctgatcagcggcaatgcccacggcaaggatgtggacgccttcatcagactgaccgtggacgacatgctgaccgacaccgagcggagaatcaagagattcaaggacgaccggaagtccattcggagcgccgacaacaagatgggaaagagaggcttcaagcagatctccacaggcaagctggccgacttcctggccaaggacatcgtgctgtttcagcccagcgtgaacgatggcgagaacaagatcaccggcctgaactaccggatcatgcagagcgccattgccgtgtacgatagcggcgacgattacgaggccaagcagcagttcaagctgatgttcgagaaggcccggctgatcggcaagggcacaacagagcctcatccatttctgtacaaggtgttcgcccgcagcatccccgccaatgccgtcgagttctacgagcgctacctgatcgagcggaagttctacctgaccggcctgtccaacgagatcaagaaaggcaacagagtggatgtgcccttcatccggcgggaccagaacaagtggaaaacacccgccatgaagaccctgggcagaatctacagcgaggatctgcccgtggaactgcccagacagatgttcgacaatgagatcaagtcccacctgaagtccctgccacagatggaaggcatcgacttcaacaatgccaacgtgacctatctgatcgccgagtacatgaagagagtgctggacgacgacttccagaccttctaccagtggaaccgcaactaccggtacatggacatgcttaagggcgagtacgacagaaagggctccctgcagcactgcttcaccagcgtggaagagagagaaggcctctggaaagagcgggcctccagaacagagcggtacagaaagcaggccagcaacaagatccgcagcaaccggcagatgagaaacgccagcagcgaagagatcgagacaatcctggataagcggctgagcaacagccggaacgagtaccagaaaagcgagaaagtgatccggcgctacagagtgcaggatgccctgctgtttctgctggccaaaaagaccctgaccgaactggccgatttcgacggcgagaggttcaaactgaaagaaatcatgcccgacgccgagaagggaatcctgagcgagatcatgcccatgagcttcaccttcgagaaaggcggcaagaagtacaccatcaccagcgagggcatgaagctgaagaactacggcgacttctttgtgctggctagcgacaagaggatcggcaacctgctggaactcgtgggcagcgacatcgtgtccaaagaggatatcatggaagagttcaacaaatacgaccagtgcaggcccgagatcagctccatcgtgttcaacctggaaaagtgggccttcgacacataccccgagctgtctgccagagtggaccgggaagagaaggtggacttcaagagcatcctgaaaatcctgctgaacaacaagaacatcaacaaagagcagagcgacatcctgcggaagatccggaacgccttcgatcacaacaattaccccgacaaaggcgtggtggaaatcaaggccctgcctgagatcgccatgagcatcaagaaggcctttggggagtacgccatcatgaag

SEQ ID NO: 9 - DNA, 53 bp, synthetic construct
  source 1..53: mol_type = other DNA
  misc_difference 13: note = a, c, t, g, unknown, or other
  misc_difference 14: note = a, c, t, g, unknown, or other
  misc_difference 15: note = a, c, t, g, unknown, or other
  misc_difference 16: note = a, c, t, g, unknown, or other
  misc_difference 17: note = a, c, t, g, unknown, or other
  misc_difference 18: note = a, c, t, g, unknown, or other
  misc_difference 19: note = a, c, t, g, unknown, or other
  misc_difference 20: note = a, c, t, g, unknown, or other
  misc_difference 21: note = a, c, t, g, unknown, or other
  misc_difference 22: note = a, c, t, g, unknown, or other
  misc_difference 23: note = a, c, t, g, unknown, or other
  misc_difference 24: note = a, c, t, g, unknown, or other
  misc_difference 25: note = a, c, t, g, unknown, or other
  misc_difference 26: note = a, c, t, g, unknown, or other
  misc_difference 27: note = a, c, t, g, unknown, or other
  misc_difference 28: note = a, c, t, g, unknown, or other
  misc_difference 29: note = a, c, t, g, unknown, or other
  misc_difference 30: note = a, c, t, g, unknown, or other
  misc_difference 31: note = a, c, t, g, unknown, or other
  misc_difference 32: note = a, c, t, g, unknown, or other
  misc_difference 33: note = a, c, t, g, unknown, or other
  misc_difference 34: note = a, c, t, g, unknown, or other
  misc_difference 35: note = a, c, t, g, unknown, or other
  misc_difference 36: note = a, c, t, g, unknown, or other
  misc_difference 37: note = a, c, t, g, unknown, or other
  misc_difference 38: note = a, c, t, g, unknown, or other
  misc_difference 39: note = a, c, t, g, unknown, or other
  misc_difference 40: note = a, c, t, g, unknown, or other
  misc_difference 41: note = a, c, t, g, unknown, or other
  misc_difference 42: note = a, c, t, g, unknown, or other
  misc_difference 43: note = a, c, t, g, unknown, or other
  misc_difference 44: note = a, c, t, g, unknown, or other
  misc_difference 45: note = a, c, t, g, unknown, or other
aagcttgcagtgnnnnnnnnnnnnnnnnnnnnnnnnnnnnnnnnntgaagagc

SEQ ID NO: 10 - DNA, 20 bp, synthetic construct
  source 1..20: mol_type = other DNA
cactgcaagcttgctcttca

SEQ ID NO: 11 - DNA, 20 bp, synthetic construct
  source 1..20: mol_type = other DNA
tgaagagcaagcttgcagtg

SEQ ID NO: 12 - DNA, 53 bp, synthetic construct
  source 1..53: mol_type = other DNA
  misc_difference 21: note = a, c, t, g, unknown, or other
  misc_difference 22: note = a, c, t, g, unknown, or other
  misc_difference 23: note = a, c, t, g, unknown, or other
  misc_difference 24: note = a, c, t, g, unknown, or other
  misc_difference 25: note = a, c, t, g, unknown, or other
  misc_difference 26: note = a, c, t, g, unknown, or other
  misc_difference 27: note = a, c, t, g, unknown, or other
  misc_difference 28: note = a, c, t, g, unknown, or other
  misc_difference 29: note = a, c, t, g, unknown, or other
  misc_difference 30: note = a, c, t, g, unknown, or other
  misc_difference 31: note = a, c, t, g, unknown, or other
  misc_difference 32: note = a, c, t, g, unknown, or other
  misc_difference 33: note = a, c, t, g, unknown, or other
  misc_difference 34: note = a, c, t, g, unknown, or other
  misc_difference 35: note = a, c, t, g, unknown, or other
  misc_difference 36: note = a, c, t, g, unknown, or other
  misc_difference 37: note = a, c, t, g, unknown, or other
  misc_difference 38: note = a, c, t, g, unknown, or other
  misc_difference 39: note = a, c, t, g, unknown, or other
  misc_difference 40: note = a, c, t, g, unknown, or other
  misc_difference 41: note = a, c, t, g, unknown, or other
  misc_difference 42: note = a, c, t, g, unknown, or other
  misc_difference 43: note = a, c, t, g, unknown, or other
  misc_difference 44: note = a, c, t, g, unknown, or other
  misc_difference 45: note = a, c, t, g, unknown, or other
  misc_difference 46: note = a, c, t, g, unknown, or other
  misc_difference 47: note = a, c, t, g, unknown, or other
  misc_difference 48: note = a, c, t, g, unknown, or other
  misc_difference 49: note = a, c, t, g, unknown, or other
  misc_difference 50: note = a, c, t, g, unknown, or other
  misc_difference 51: note = a, c, t, g, unknown, or other
  misc_difference 52: note = a, c, t, g, unknown, or other
  misc_difference 53: note = a, c, t, g, unknown, or other
cactgcaagcttgctcttcannnnnnnnnnnnnnnnnnnnnnnnnnnnnnnnn

SEQ ID NO: 13 - DNA, 86 bp, synthetic construct
  source 1..86: mol_type = other DNA
  misc_difference 1: note = a, c, t, g, unknown, or other
  misc_difference 2: note = a, c, t, g, unknown, or other
  misc_difference 3: note = a, c, t, g, unknown, or other
  misc_difference 4: note = a, c, t, g, unknown, or other
  misc_difference 5: note = a, c, t, g, unknown, or other
  misc_difference 6: note = a, c, t, g, unknown, or other
  misc_difference 7: note = a, c, t, g, unknown, or other
  misc_difference 8: note = a, c, t, g, unknown, or other
  misc_difference 9: note = a, c, t, g, unknown, or other
  misc_difference 10: note = a, c, t, g, unknown, or other
  misc_difference 11: note = a, c, t, g, unknown, or other
  misc_difference 12: note = a, c, t, g, unknown, or other
  misc_difference 13: note = a, c, t, g, unknown, or other
  misc_difference 14: note = a, c, t, g, unknown, or other
  misc_difference 15: note = a, c, t, g, unknown, or other
  misc_difference 16: note = a, c, t, g, unknown, or other
  misc_difference 17: note = a, c, t, g, unknown, or other
  misc_difference 18: note = a, c, t, g, unknown, or other
  misc_difference 19: note = a, c, t, g, unknown, or other
  misc_difference 20: note = a, c, t, g, unknown, or other
  misc_difference 21: note = a, c, t, g, unknown, or other
  misc_difference 22: note = a, c, t, g, unknown, or other
  misc_difference 23: note = a, c, t, g, unknown, or other
  misc_difference 24: note = a, c, t, g, unknown, or other
  misc_difference 25: note = a, c, t, g, unknown, or other
  misc_difference 26: note = a, c, t, g, unknown, or other
  misc_difference 27: note = a, c, t, g, unknown, or other
  misc_difference 28: note = a, c, t, g, unknown, or other
  misc_difference 29: note = a, c, t, g, unknown, or other
  misc_difference 30: note = a, c, t, g, unknown, or other
  misc_difference 31: note = a, c, t, g, unknown, or other
  misc_difference 32: note = a, c, t, g, unknown, or other
  misc_difference 33: note = a, c, t, g, unknown, or other
  misc_difference 54: note = a, c, t, g, unknown, or other
  misc_difference 55: note = a, c, t, g, unknown, or other
  misc_difference 56: note = a, c, t, g, unknown, or other
  misc_difference 57: note = a, c, t, g, unknown, or other
  misc_difference 58: note = a, c, t, g, unknown, or other
  misc_difference 59: note = a, c, t, g, unknown, or other
  misc_difference 60: note = a, c, t, g, unknown, or other
  misc_difference 61: note = a, c, t, g, unknown, or other
  misc_difference 62: note = a, c, t, g, unknown, or other
  misc_difference 63: note = a, c, t, g, unknown, or other
  misc_difference 64: note = a, c, t, g, unknown, or other
  misc_difference 65: note = a, c, t, g, unknown, or other
  misc_difference 66: note = a, c, t, g, unknown, or other
  misc_difference 67: note = a, c, t, g, unknown, or other
  misc_difference 68: note = a, c, t, g, unknown, or other
  misc_difference 69: note = a, c, t, g, unknown, or other
  misc_difference 70: note = a, c, t, g, unknown, or other
  misc_difference 71: note = a, c, t, g, unknown, or other
  misc_difference 72: note = a, c, t, g, unknown, or other
  misc_difference 73: note = a, c, t, g, unknown, or other
  misc_difference 74: note = a, c, t, g, unknown, or other
  misc_difference 75: note = a, c, t, g, unknown, or other
  misc_difference 76: note = a, c, t, g, unknown, or other
  misc_difference 77: note = a, c, t, g, unknown, or other
  misc_difference 78: note = a, c, t, g, unknown, or other
  misc_difference 79: note = a, c, t, g, unknown, or other
  misc_difference 80: note = a, c, t, g, unknown, or other
  misc_difference 81: note = a, c, t, g, unknown, or other
  misc_difference 82: note = a, c, t, g, unknown, or other
  misc_difference 83: note = a, c, t, g, unknown, or other
  misc_difference 84: note = a, c, t, g, unknown, or other
  misc_difference 85: note = a, c, t, g, unknown, or other
  misc_difference 86: note = a, c, t, g, unknown, or other
nnnnnnnnnnnnnnnnnnnnnnnnnnnnnnnnncactgcaagcttgctcttcannnnnnnnnnnnnnnnnnnnnnnnnnnnnnnnn

SEQ ID NO: 14 - DNA, 53 bp, synthetic construct
  source 1..53: mol_type = other DNA
  misc_difference 14: note = a, c, t, g, unknown, or other
  misc_difference 15: note = a, c, t, g, unknown, or other
  misc_difference 16: note = a, c, t, g, unknown, or other
  misc_difference 17: note = a, c, t, g, unknown, or other
  misc_difference 18: note = a, c, t, g, unknown, or other
  misc_difference 19: note = a, c, t, g, unknown, or other
  misc_difference 20: note = a, c, t, g, unknown, or other
  misc_difference 21: note = a, c, t, g, unknown, or other
  misc_difference 22: note = a, c, t, g, unknown, or other
  misc_difference 23: note = a, c, t, g, unknown, or other
  misc_difference 24: note = a, c, t, g, unknown, or other
  misc_difference 25: note = a, c, t, g, unknown, or other
  misc_difference 26: note = a, c, t, g, unknown, or other
  misc_difference 27: note = a, c, t, g, unknown, or other
  misc_difference 28: note = a, c, t, g, unknown, or other
  misc_difference 29: note = a, c, t, g, unknown, or other
  misc_difference 30: note = a, c, t, g, unknown, or other
  misc_difference 31: note = a, c, t, g, unknown, or other
  misc_difference 32: note = a, c, t, g, unknown, or other
  misc_difference 33: note = a, c, t, g, unknown, or other
  misc_difference 34: note = a, c, t, g, unknown, or other
  misc_difference 35: note = a, c, t, g, unknown, or other
  misc_difference 36: note = a, c, t, g, unknown, or other
  misc_difference 37: note = a, c, t, g, unknown, or other
  misc_difference 38: note = a, c, t, g, unknown, or other
  misc_difference 39: note = a, c, t, g, unknown, or other
  misc_difference 40: note = a, c, t, g, unknown, or other
  misc_difference 41: note = a, c, t, g, unknown, or other
  misc_difference 42: note = a, c, t, g, unknown, or other
  misc_difference 43: note = a, c, t, g, unknown, or other
  misc_difference 44: note = a, c, t, g, unknown, or other
  misc_difference 45: note = a, c, t, g, unknown, or other
  misc_difference 46: note = a, c, t, g, unknown, or other
agcttgctcttcannnnnnnnnnnnnnnnnnnnnnnnnnnnnnnnncactgca

SEQ ID NO: 15 - DNA, 53 bp, synthetic construct
  source 1..53: mol_type = other DNA
  misc_difference 21: note = a, c, t, g, unknown, or other
  misc_difference 22: note = a, c, t, g, unknown, or other
  misc_difference 23: note = a, c, t, g, unknown, or other
  misc_difference 24: note = a, c, t, g, unknown, or other
  misc_difference 25: note = a, c, t, g, unknown, or other
  misc_difference 26: note = a, c, t, g, unknown, or other
  misc_difference 27: note = a, c, t, g, unknown, or other
  misc_difference 28: note = a, c, t, g, unknown, or other
  misc_difference 29: note = a, c, t, g, unknown, or other
  misc_difference 30: note = a, c, t, g, unknown, or other
  misc_difference 31: note = a, c, t, g, unknown, or other
  misc_difference 32: note = a, c, t, g, unknown, or other
  misc_difference 33: note = a, c, t, g, unknown, or other
  misc_difference 34: note = a, c, t, g, unknown, or other
  misc_difference 35: note = a, c, t, g, unknown, or other
  misc_difference 36: note = a, c, t, g, unknown, or other
  misc_difference 37: note = a, c, t, g, unknown, or other
  misc_difference 38: note = a, c, t, g, unknown, or other
  misc_difference 39: note = a, c, t, g, unknown, or other
  misc_difference 40: note = a, c, t, g, unknown, or other
  misc_difference 41: note = a, c, t, g, unknown, or other
  misc_difference 42: note = a, c, t, g, unknown, or other
  misc_difference 43: note = a, c, t, g, unknown, or other
  misc_difference 44: note = a, c, t, g, unknown, or other
  misc_difference 45: note = a, c, t, g, unknown, or other
  misc_difference 46: note = a, c, t, g, unknown, or other
  misc_difference 47: note = a, c, t, g, unknown, or other
  misc_difference 48: note = a, c, t, g, unknown, or other
  misc_difference 49: note = a, c, t, g, unknown, or other
  misc_difference 50: note = a, c, t, g, unknown, or other
  misc_difference 51: note = a, c, t, g, unknown, or other
  misc_difference 52: note = a, c, t, g, unknown, or other
  misc_difference 53: note = a, c, t, g, unknown, or other
tgaagagcaagcttgcagtgnnnnnnnnnnnnnnnnnnnnnnnnnnnnnnnnn

SEQ ID NO: 16 - DNA, 86 bp, synthetic construct
  source 1..86: mol_type = other DNA
  misc_difference 1: note = a, c, t, g, unknown, or other
  misc_difference 2: note = a, c, t, g, unknown, or other
  misc_difference 3: note = a, c, t, g, unknown, or other
  misc_difference 4: note = a, c, t, g, unknown, or other
  misc_difference 5: note = a, c, t, g, unknown, or other
  misc_difference 6: note = a, c, t, g, unknown, or other
  misc_difference 7: note = a, c, t, g, unknown, or other
  misc_difference 8: note = a, c, t, g, unknown, or other
  misc_difference 9: note = a, c, t, g, unknown, or other
  misc_difference 10: note = a, c, t, g, unknown, or other
  misc_difference 11: note = a, c, t, g, unknown, or other
  misc_difference 12: note = a, c, t, g, unknown, or other
  misc_difference 13: note = a, c, t, g, unknown, or other
  misc_difference 14: note = a, c, t, g, unknown, or other
  misc_difference 15: note = a, c, t, g, unknown, or other
  misc_difference 16: note = a, c, t, g, unknown, or other
  misc_difference 17: note = a, c, t, g, unknown, or other
  misc_difference 18: note = a, c, t, g, unknown, or other
  misc_difference 19: note = a, c, t, g, unknown, or other
  misc_difference 20: note = a, c, t, g, unknown, or other
  misc_difference 21: note = a, c, t, g, unknown, or other
  misc_difference 22: note = a, c, t, g, unknown, or other
  misc_difference 23: note = a, c, t, g, unknown, or other
  misc_difference 24: note = a, c, t, g, unknown, or other
  misc_difference 25: note = a, c, t, g, unknown, or other
  misc_difference 26: note = a, c, t, g, unknown, or other
  misc_difference 27: note = a, c, t, g, unknown, or other
  misc_difference 28: note = a, c, t, g, unknown, or other
  misc_difference 29: note = a, c, t, g, unknown, or other
  misc_difference 30: note = a, c, t, g, unknown, or other
  misc_difference 31: note = a, c, t, g, unknown, or other
  misc_difference 32: note = a, c, t, g, unknown, or other
  misc_difference 33: note = a, c, t, g, unknown, or other
  misc_difference 54: note = a, c, t, g, unknown, or other
  misc_difference 55: note = a, c, t, g, unknown, or other
  misc_difference 56: note = a, c, t, g, unknown, or other
  misc_difference 57: note = a, c, t, g, unknown, or other
  misc_difference 58: note = a, c, t, g, unknown, or other
  misc_difference 59: note = a, c, t, g, unknown, or other
  misc_difference 60: note = a, c, t, g, unknown, or other
  misc_difference 61: note = a, c, t, g, unknown, or other
  misc_difference 62: note = a, c, t, g, unknown, or other
  misc_difference 63: note = a, c, t, g, unknown, or other
  misc_difference 64: note = a, c, t, g, unknown, or other
  misc_difference 65: note = a, c, t, g, unknown, or other
  misc_difference 66: note = a, c, t, g, unknown, or other
  misc_difference 67: note = a, c, t, g, unknown, or other
  misc_difference 68: note = a, c, t, g, unknown, or other
  misc_difference 69: note = a, c, t, g, unknown, or other
  misc_difference 70: note = a, c, t, g, unknown, or other
  misc_difference 71: note = a, c, t, g, unknown, or other
  misc_difference 72: note = a, c, t, g, unknown, or other
  misc_difference 73: note = a, c, t, g, unknown, or other
  misc_difference 74: note = a, c, t, g, unknown, or other
  misc_difference 75: note = a, c, t, g, unknown, or other
  misc_difference 76: note = a, c, t, g, unknown, or other
  misc_difference 77: note = a, c, t, g, unknown, or other
  misc_difference 78: note = a, c, t, g, unknown, or other
  misc_difference 79: note = a, c, t, g, unknown, or other
  misc_difference 80: note = a, c, t, g, unknown, or other
  misc_difference 81: note = a, c, t, g, unknown, or other
  misc_difference 82: note = a, c, t, g, unknown, or other
  misc_difference 83: note = a, c, t, g, unknown, or other
  misc_difference 84: note = a, c, t, g, unknown, or other
  misc_difference 85: note = a, c, t, g, unknown, or other
  misc_difference 86: note = a, c, t, g, unknown, or other
nnnnnnnnnnnnnnnnnnnnnnnnnnnnnnnnntgaagagcaagcttgcagtgnnnnnnnnnnnnnnnnnnnnnnnnnnnnnnnnn

SEQ ID NO: 17 - DNA, 53 bp, synthetic construct
  source 1..53: mol_type = other DNA
  misc_difference 12: note = a, c, t, g, unknown, or other
  misc_difference 13: note = a, c, t, g, unknown, or other
  misc_difference 14: note = a, c, t, g, unknown, or other
  misc_difference 15: note = a, c, t, g, unknown, or other
  misc_difference 16: note = a, c, t, g, unknown, or other
  misc_difference 17: note = a, c, t, g, unknown, or other
  misc_difference 18: note = a, c, t, g, unknown, or other
  misc_difference 19: note = a, c, t, g, unknown, or other
  misc_difference 20: note = a, c, t, g, unknown, or other
  misc_difference 21: note = a, c, t, g, unknown, or other
  misc_difference 22: note = a, c, t, g, unknown, or other
  misc_difference 23: note = a, c, t, g, unknown, or other
  misc_difference 24: note = a, c, t, g, unknown, or other
  misc_difference 25: note = a, c, t, g, unknown, or other
  misc_difference 26: note = a, c, t, g, unknown, or other
  misc_difference 27: note = a, c, t, g, unknown, or other
  misc_difference 28: note = a, c, t, g, unknown, or other
  misc_difference 29: note = a, c, t, g, unknown, or other
  misc_difference 30: note = a, c, t, g, unknown, or other
  misc_difference 31: note = a, c, t, g, unknown, or other
  misc_difference 32: note = a, c, t, g, unknown, or other
  misc_difference 33: note = a, c, t, g, unknown, or other
  misc_difference 34: note = a, c, t, g, unknown, or other
  misc_difference 35: note = a, c, t, g, unknown, or other
  misc_difference 36: note = a, c, t, g, unknown, or other
  misc_difference 37: note = a, c, t, g, unknown, or other
  misc_difference 38: note = a, c, t, g, unknown, or other
  misc_difference 39: note = a, c, t, g, unknown, or other
  misc_difference 40: note = a, c, t, g, unknown, or other
  misc_difference 41: note = a, c, t, g, unknown, or other
  misc_difference 42: note = a, c, t, g, unknown, or other
  misc_difference 43: note = a, c, t, g, unknown, or other
  misc_difference 44: note = a, c, t, g, unknown, or other
agcttgcagtgnnnnnnnnnnnnnnnnnnnnnnnnnnnnnnnnntgaagagca

SEQ ID NO: 18 - DNA, 11 bp, synthetic construct
  source 1..11: mol_type = other DNA
agcttgcagtg

SEQ ID NO: 19 - protein, 6 aa, synthetic construct
KTKEII

SEQ ID NO: 20 - protein, 8 aa, synthetic construct
DIFSKIKI

SEQ ID NO: 21 - protein, 6 aa, synthetic construct
ALEAAA

SEQ ID NO: 22 - protein, 8 aa, synthetic construct
EAELAAAT